(19) United States
(12) Patent Application Publication
(10) Pub. No.: US 2025/0346900 A1
(43) Pub. Date: Nov. 13, 2025

(54) PHARMACEUTICAL COMPOSITION FOR TREATING CANCER COMPRISING FOXM1 MUTANT OR FOXM1 SHRNA

(71) Applicant: Industry-University Cooperation Foundation Hanyang University Erica Campus, Ansan-si, Gyeonggi-do (KR)

(72) Inventors: Rong XU, Ansan-si, Gyeonggi-do (KR); Hay Ran JANG, Ansan-si, Gyeonggi-do (KR); Hyung Shin YIM, Seoul (KR)

(21) Appl. No.: 19/208,484

(22) Filed: May 14, 2025

Related U.S. Application Data
(63) Continuation of application No. PCT/KR2023/014663, filed on Sep. 25, 2023.

(30) Foreign Application Priority Data
Nov. 16, 2022 (KR) 10-2022-0153798
Nov. 22, 2022 (KR) 10-2022-0156930

(51) Int. Cl. C12N 15/113; A61K 38/17; A61P 35/00; C12N 5/09; C12N 15/86; C12Q 1/6886

(52) U.S. Cl. CPC C12N 15/1135; A61K 38/1709; A61P 35/00; C12N 5/0693; C12N 15/86; C12Q 1/6886; C12N 2310/14; C12N 2310/531; C12N 2740/15043; C12Q 2600/118

(57) ABSTRACT

The present invention relates to a use of a substance comprising point-mutated FoxM1 protein and peptide for inhibiting growth, migration and invasion of cancer cells. In addition, it relates to a use of a substance comprising point-mutated FoxM1 protein and peptide for inhibiting malignancy in a tumor microenvironment by inhibiting polarization into tumor-associated macrophages in a tumor microenvironment. It relates to a use as an apoptotic inducer using the action of sensitively inhibiting cancer cell death in metastatic cancer cells as well as general solid cancers.

### TECHNICAL FIELD

The present invention relates to a pharmaceutical composition for inhibiting cancer cell growth and invasion and metastasis using a point mutation of FoxM1 or a peptide comprising a FoxM1 point mutation, or an shRNA that specifically binds to FoxM1.

### BACKGROUND ART

Cancer can be classified according to its stage of progression, and particularly the metastasis of cancer, depending on the stage, can be an important criterion for determining the treatment method. Although the size of the cancer is important, the treatment strategy should be reviewed by dividing the cancer into primary cancer and metastatic cancer that has spread to the surrounding lymph nodes or organs. As is well known, even if the primary cancer is treated, if metastasis cannot be prevented, the survival rate becomes very low. Although it varies depending on the type of cancer, metastatic cancer can account for up to 90% of deaths of cancer patients, and thus, the prognosis is quite poor (Dillekas, et al., Cancer Med 8 (2019) 5574-5576; Khan, I. and Steeg, P. S., Lab Invest 98 (2018) 198-210). As cancer grows, it stimulates the secretion of cytokines that promote malignancy and metastasis of cancer tissues from surrounding lymph nodes and tissues, and ultimately, it can act as a cause of increasing cancer metastasis. Macrophages that have a function for eliminating cancers are polarized into tumor-associated macrophages (TAM) by IL-6, VEGFA, TGFB1, etc., secreted by cancer cells, and act as cells that assist the survival of cancers (Murray P. J. Annu Rev. Physiol. (2017) 79:541-566; Jeon S H et al., J. Leukoc Biol. (2007) 81 (2) 557-566; Wheeler K C et al., PLOS One (2018) 13 (1): e0191040). It is known that the larger the tumor size, the higher the metastasis rate. However, there are cases where metastasis occurs even when the tumor size is small. Thus, the correlation between cancer proliferation and metastasis has not yet been clearly described (Valastyan, S. and Weinberg, R. A., Cell 147 (2011) 275-292; Shibue, T. and Weinberg, Semin Cancer Biol 21 (2011) 99-106). In anti-cancer treatment, the basic goal is to suppress the proliferation of cancer cells. However, considering that the prognosis is significantly lowered if the metastasis cannot be prevented, it is necessary to develop anti-cancer treatment that effectively suppresses metastasis and invasion of cancer cells for the treatment of metastatic cancer.

FoxM1 belongs to a large family of Forkhead box (Fox) transcription factors and is a transcription regulator that has a common DNA binding domain called ‘Winged-helix” (Kaufmann, E. and Knöchel, W., Mech Dev 57 (1996) 3-20). The transcription factor FoxM1 has essential roles in the regulation of a wide range of biological processes, including cell proliferation, cell-cycle progression, cell polarization, DNA damage repair, tissue homeostasis, angiogenesis, and cell death. FoxMl is known to play a crucial role in cell-cycle progression, with its expression peaking in the S phase and G2/M phase of the cell cycle (Laoukili, J. et al., Nat Cell Biol 7 (2005) 126-136). It regulates the expression of G2/M-specific genes, such as PLK1, cyclin B1, Nek2, and CENPF (WANG, I.-Ching et al., Mol Cell Biol (2005) 25.24:10875-10894). Furthermore, the mitosis transcription factor FoxM1 is an EMT regulator presumed by activation of EMT transcription factors, including SNAIL and SNAI2. It can stimulate the expression of genes involved in various stages of tumor metastasis, including epithelial-to-mesenchymal-like transition, cell migration, and metastatic niche formation. The increased expression of FoxM1 is observed in various malignant cancers, such as liver cancer (YU, Chun-Peng et al. Molecular medicine reports 16.4 (2017) 5181-5188), prostate cancer (Kalin, Tanya V. et al., Cancer research 66.3 (2006) 1712-1720), colorectal cancer (Yoshida, Yuichi et al., Gastroenterology 132.4 (2007) 1420-1431), brain cancer (Liu, Mingguang et al., Cancer research 66.7 (2006) 3593-3602), breast cancer (Millour,

Julie and E. W. Lam. Breast Cancer Research 12.1 (2010) 1-1), lung cancer (Wang, I-Ching et al. PLOS One 4.8 (2009): e6609), colon cancer, pancreatic cancer, skin cancer, cervical cancer, ovarian cancer, oral cancer, blood cancer, and nervous system cancer (BARGER, Carter J. et al., Cancers (2019) 11.2:251). Genome-wide gene expression profiling of cancers has independently and consistently identified FoxM1 as one of the most commonly upregulated genes in human solid tumors. These findings suggest that FoxM1 plays a key role in tumorigenesis.

The expression of FoxM1 increases in proliferating and dividing cells functionally, and especially, its expression and activity peak during the mitotic phase of the cell cycle. Accordingly, it is also known to have a high expression rate in fast-growing cancer cells (Liao, Guo-Bin et al., Cell Communication and Signaling 16.1 (2018): 1-15). Recently, as it has been reported that the expression is increased in various cancer types according to the stages, its relevance in cancer metastasis has been suggested in addition to its function in driving growth. In particular, it has been reported that its expression increases as the stage advances in colorectal cancer (Fei, Bao-Ying et al., Oncology Letters 14.6 (2017): 6553-6561), lung cancer (Wei, Ping et al., Int J Biol Sci 11.2 (2015): 186), and ovarian cancer (Chan, David W. et al., Oncogene 36.10 (2017): 1404-1416) (Li, Lijun et al., Oncotarget 8.19 (2017): 32298). Therefore, if the expression of FoxM1, which is highly expressed in high-stage cancer, is suppressed, it is expected to be highly effective in suppressing the growth of primary cancer and the transformation into metastatic cancer.

Depending on the cancer types, metastatic cancer accounts for 90% of cancer patient deaths, and thus its risk to patient survival rate is well known (Valastyan, S. and Weinberg, R. A., Cell 147 (2011) 275-292; Khan, I. and Steeg, P. S., Lab Invest 98 (2018) 198-210). Metastasis is a phenomenon in which primary cancer cells migrate to environments suitable for survival, and if the metastasis cannot be prevented, the removal of the primary cancer alone leads to significantly lowered survival rates in patients due to cancer recurrence and other factors. Although it is known that the rate of metastasis to the surrounding lymph nodes and organs increases as the cancer size increases, there are cases where metastasis occurs despite the cancer being small. Therefore, the relationship between cancer metastasis and proliferation has not yet been clearly elucidated (Valastyan, S. and Weinberg, R. A., Cell 147 (2011) 275-292; Shibue, T. and Weinberg, Semin Cancer Biol 21 (2011) 99-106).

Polo-like kinase 1 (PLK1) is a representative cell-dividing factor regulating cell growth, and it is used for the diagnosis of various cancers due to its high expression in various solid cancers and blood cancers. Recently, it has been reported that PLK1 can induce metastasis as well as carcinogenesis (Shin et al., Oncogene (2020) 39 (4) 767-785; Wu et al., Elife (2016) doi: 10.7554/eLife.10734.), and thus it is investigated as a target molecule for metastasis. Based on these characteristics of PLK1, the research on developing anti-cancer drugs through the development of PLK1 inhibitors is conducted competitively, mainly by multinational companies (Yim, Anti-Cancer Drugs 24 (2013) 999-1006; Zhang, J. Med. Chem. 65 (2022) 10133-10160). Structurally, PLK1 involves a kinase catalytic domain that contains an ATP-binding domain capable of binding to ATP and a polo-box domain that binds to substrates. PLK1 activation is induced by phosphorylation of Thr210 residue in the kinase catalytic domain, and PLK1 is the enzyme that phosphorylates Ser/Thr residues of substrates bound to the polo-box domain (Barr et al., Nat Rev Mol Cell Biol 5 (2004) 429-440). Functionally, PLK1 expression increases during cell growth and division, and is especially highest during cell division, with its activity reaching its peak. Hence, its expression rate is also high in rapidly growing cancer cells (Yim and Erikson, Mutation Research Reviews Mutation Research, 761 (2014) 31-39; Barr et al., Nat Rev Mol Cell Biol 5 (2004) 429-440). In recent studies, it has been reported based on clinical results that in the stage-by-stage malignant process of various cancers, most of the PLK1 expression increases as the stage advances. In particular, it has been reported that its expression increases as the malignancy stage advances in prostate cancer, non-small cell lung cancer, endometrial cancer, colorectal cancer, ovarian cancer, laryngeal cancer, and others (Yim and Erikson, Mutation Research Reviews Mutation Research, 761 (2014) 31-39; Kim et al., Exp Mol Med, 54 (2022) 414-425). According to recent studies, it has been reported that even the active form of PLKI alone involves in promoting the cancer metastasis (Shin et al., Oncogene (2020) 39 (4) 767-785; Cai et al., Am J Transl Res 8 (2016) 4172-4183; Wu et al., Elife (2016) doi: 10.7554/eLife.10734.), and based on these studies, targeting against PLK1 is being magnified as a therapeutic strategy for treatment of metastatic cancer. Therefore, these researchers have constructed the development strategies for anti-cancer drugs for metastatic cancers as well as primary cancers through regulating the functions of substrates that are directly phosphorylated by PLK1.

The process of metastasis is a multi-step process, and it is known that the initial invasion and motility are acquired through the epithelial-mesenchymal transition (EMT) process of cancer cells (Dongre, Anushka, and Robert A. Weinberg. Nature reviews Molecular cell biology 20.2 (2019): 69-84). EMT is a process in which epithelial cells lose their properties, such as tight intercellular adhesion, and transform into mesenchymal cells with motility and invasion. During the process, when observing changes in intracellular factors, it is observed that the epithelial marker, E-cadherin, is decreased, while the mesenchymal markers, such as N-cadherin, vimentin, SNAI1, and SNAI2, are increased. In the treatment of cancer, the suppression of cancer cell proliferation and the suppression of metastasis do not always occur simultaneously. Considering that suppression of metastasis can dramatically improve the therapeutic efficiency for many cancers, it is necessary to develop therapeutic targets or drugs that can effectively inhibit metastasis and invasion for the treatment of metastatic cancers.

The present inventors have developed phosphorylation site mutants of FoxM1 targeted by PLK1, and, through efforts to develop gene-and peptide-based anticancer therapies based thereon, have discovered that a non-phosphorylated point mutant at the PLK1 phosphorylation site Ser25 of FoxM1 exhibits an inhibitory effect on metastasis and invasion of lung cancer cells. In addition, it has been confirmed that the FoxM1 phosphorylated point mutant at Ser25 promoted polarization into tumor-associated macrophages assisting tumor survival by moving monocytes present in the tumor microenvironment to adjacent to tumor, increased the expression of VEGFA, which is a major factor for angiogenesis, and induced malignancy in a series of tumor microenvironments that assist T cells to evade cancer cells through immune evasion, thereby it have been reversely used. The present invention has been completed by confirming that it could be used for treatment of metastatic cancer by blocking the metastasis, invasion, and tumorigenesis of various solid tumors, and by reducing the activity of immune cells that help tumors evade immunity in the tumor microenvironment through developing the non-phosphorylated point mutant protein and peptide of FoxM1.

In addition, in order to block the action of FoxM1 that promoted polarization into tumor-associated macrophages in the tumor microenvironment as well as the regulation of tumor growth and metastasis, FoxM1 shRNA for inhibiting FoxM1 expression and thiostepton as a FoxM1 inhibitor have been used, and it have been found the results of FoxM1 shRNA for inhibiting FoxM1 expression and thiostepton as a FoxM1 inhibitor significantly reducing metastasis, invasion, and polarization into tumor-associated macrophages in lung cancer, and thereby the present inventors have completed the present invention by confirming that FoxMl shRNA and FoxM1 inhibitor thiostepton can be useful for treatment of metastatic cancer by blocking and reducing the metastasis, invasion, and tumor formation of various solid tumors.

### DISCLOSURE

### Technical Problem

The present invention is to provide a therapeutic agent for cancer, in which FoxM1 protein and peptide comprising a point mutation, or an shRNA that can inhibit expression FOXM1 by specifically binding to FOXM1 inhibits growth and invasion and metastasis, and which uses the strong inhibitory action on the activity of immune cells that help metastasis and invasion of cancer cells, and immune evasion of tumor in a tumor microenvironment shown in lung cancer cells by the FoxM1 protein comprising a non-phosphorylated point mutation or a fragment thereof, or an shRNA that specifically binds to FOXM1.

### Technical Solution

The present invention provides a pharmaceutical composition for treating cancer, comprising a polypeptide in which the 25th amino acid, Ser of SEQ ID NO: 1 is substituted with a non-phosphorylated amino acid. In one example of the present invention, the non-phosphorylated amino acid is Gly, Ala, Val, Ile, Leu, Met, Phe, Trp, Asn, Gln, Cys, Pro, Arg, His, or Lys, and in another example of the present invention, the cancer is bone cancer, blood cancer, lung cancer, small cell lung cancer, non-small cell lung cancer, squamous cell carcinoma, adenocarcinoma, large cell lung cancer, liver cancer, pancreatic cancer, skin cancer, head and neck cancer, skin or intraocular melanoma, uterine cancer, ovarian cancer, rectal cancer, anal region cancer, gastric cancer, colorectal cancer, breast cancer, prostate cancer, endometrial cancer, sarcoma, pheochromocytoma adrenal tumor, testicular germ cell tumor, cervical cancer, carcinoma of sexual and reproductive organs, Hodgkin's disease, esophageal cancer, small intestinal cancer, endocrine cancer, thyroid cancer, parathyroid cancer, adrenal cancer, soft tissue sarcoma, bladder cancer, kidney cancer, renal cell carcinoma, renal pelvic carcinoma, central nervous system (CNS) neoplasm, neuroectodermal tumor, spinal tumor, glioma, meningioma or pituitary adenoma, and in other example of the present invention, the pharmaceutical composition inhibits at least one selected from the group consisting of growth, migration, invasion and metastasis of cancer cells.

In one example of the present invention, provided is a polypeptide, comprising a polypeptide, comprising the 24th amino acid, Pro to the 27th amino acid, Thr of SEQ ID NO: 1, and comprising 10 or more consecutive amino acids, in which the 25th amino acid, Ser is substituted with a non-phosphorylated amino acid, and in another example of the present invention, the polypeptide comprises an amino acid sequence represented by SEQ ID NO: 2. 4 or 5.

In one example of the present invention, provided is a pharmaceutical composition for treating cancer, comprising the 24th amino acid, Pro to the 27th amino acid, Thr of SEQ ID NO: 1, and comprising 10 or more consecutive amino acids, in which the 25th amino acid, Ser is substituted with a non-phosphorylated amino acid. In one example of the present invention, the polypeptide comprises an amino acid sequence represented by SEQ ID NO: 2. 4 or 5, and in another example of the present invention, the cancer is bone cancer, blood cancer, lung cancer, small cell lung cancer, non-small cell lung cancer, squamous cell carcinoma, adenocarcinoma, large cell lung cancer, liver cancer, pancreatic cancer, skin cancer, head and neck cancer, skin or intraocular melanoma, uterine cancer, ovarian cancer, rectal cancer, anal region cancer, gastric cancer, colorectal cancer, breast cancer, prostate cancer, endometrial cancer, sarcoma, pheochromocytoma adrenal tumor, testicular germ cell tumor, cervical cancer, carcinoma of sexual and reproductive organs, Hodgkin's disease, esophageal cancer, small intestinal cancer, endocrine cancer, thyroid cancer, parathyroid cancer, adrenal cancer, soft tissue sarcoma, bladder cancer, kidney cancer, renal cell carcinoma, renal pelvic carcinoma, central nervous system (CNS) neoplasm, neuroectodermal tumor, spinal tumor, glioma, meningioma or pituitary adenoma, and in other example of the present invention, the pharmaceutical composition inhibits at least one selected from the group consisting of growth, migration, invasion and metastasis of cancer cells.

In one example of the present invention, provided is a nucleic acid molecule encoding a polypeptide in which the 25th amino acid, Ser of SEQ ID NO: 1 is substituted with a non-phosphorylated amino acid, or the polypeptide comprising a polypeptide, comprising the 24th amino acid, Pro to the 27th amino acid, Thr of SEQ ID NO: 1, and comprising 10 or more consecutive amino acids, in which the 25th amino acid, Ser is substituted with a non-phosphorylated amino acid, or a polypeptide comprising an amino acid sequence represented by SEQ ID NO: 2, 4, or 5, and in another example of the present invention, the nucleic acid molecule is a nucleic acid molecule in which the 73th nucleic acid to the 75th nucleic acid represented by SEQ ID NO: 3 are substituted with 5′-GAT-3′, 5′-GAC-3′, 5′-GAA-3′, or 5′-GAG-3′; a nucleic acid molecule in which the 73th nucleic acid to the 75th nucleic acid represented by SEQ ID NO: 3 are substituted with 5′-GAT-3′, 5′-GAC-3′, 5′-GAA-3′, or 5′-GAG-3′, comprising the 61th nucleic acid to the 90th nucleic acid sequence; a nucleic acid molecule in which the 73th nucleic acid to the 75th nucleic acid represented by SEQ ID NO: 3 are substituted with 5′-GAT-3′, 5′-GAC-3′, 5′-GAA-3′, or 5′-GAG-3′, comprising the 70th nucleic acid to the 99th nucleic acid sequence; a nucleic acid molecule in which the 73th nucleic acid to the 75th nucleic acid represented by SEQ

ID NO: 3 are substituted with 5′-GAT-3′, 5′-GAC-3′, 5′-GAA-3′, or 5′-GAG-3′, comprising the 52th nucleic acid to the 81th nucleic acid sequence.

In one example of the present invention, a recombinant vector comprising the nucleic acid molecule is provided.

In one example of the present invention, a recombinant cell comprising the recombinant vector is provided.

In one example of the present invention, provided is a method of providing information for determining a risk of metastasis of cancer, comprising determining that there is a high possibility of metastasis of cancer, when the 25th amino acid, Ser of the FoxM1 protein represented by SEQ ID NO: 1 is phosphorylated or substituted with Asp or Glu, in cancer cells of a subject.

In another example of the present invention, provided is a method of providing information for determining a risk of metastasis of cancer, comprising determining that there is a high possibility of metastasis of cancer, when the 73th nucleic acid to the 75th nucleic acid in the nucleic acid sequence represented by SEQ ID NO: 3 are substituted with 5′-GAT-3′, 5′-GAC-3′, 5′-GAA-3′, or 5′-GAG-3′, in cancer cells of a subject.

In one example of the present invention, provided is a recombinant metastatic cancer cell that can be used for studying metastatic cancer, which expresses a polypeptide in which the 25th amino acid, Ser of SEQ ID NO: 1 is substituted with Asp, or Glu.

In one example of the present invention, provided is a recombinant metastatic cancer cell that can be used for studying metastatic cancer, which comprises a nucleic acid in which the 73th to the 75th nucleic acids of the nucleic acid sequence represented by SEQ ID NO: 3 are substituted with 5′-GAT-3′, 5′-GAC-3′, 5′-GAA-3′, or 5′-GAG-3′.

In one example of the present invention, provided is a pharmaceutical composition for treating cancer, comprising a nucleic acid molecule which complementarily binds to a gene or mRNA encoding a FOXM1 protein to reduce expression of the FOXM1 protein. In one example of the present invention, the gene or mRNA encoding the FOXM1 protein comprises a nucleic acid sequence represented by SEQ ID NO: 6, and in another example of the present invention, the nucleic acid molecule comprises a nucleic acid sequence that specifically binds to the 187th to 207th nucleic acids or the 709th to the 729th nucleic acids of SEQ ID NO: 6, and in other example of the present invention, the nucleic acid molecule comprises any one nucleic acid sequence of SEQ ID NOs: 7 to 10, and in other example of the present invention, the nucleic acid molecule is any one selected from the group consisting of shRNA, siRNA, antisense RNA, antisense DNA, chimeric antisense DNA/RNA, miRNA, and ribozyme, and in other example of the present invention, the cancer is cancer which overexpresses a FOXM1 protein, or expresses a FOXM1 protein in which the 25th amino acid, Ser is substituted with Asp, or Glu, and in other example of the present invention, the cancer is bone cancer, blood cancer, lung cancer, small cell lung cancer, non-small cell lung cancer, squamous cell carcinoma, adenocarcinoma, large cell lung cancer, liver cancer, pancreatic cancer, skin cancer, head and neck cancer, skin or intraocular melanoma, uterine cancer, ovarian cancer, rectal cancer, anal region cancer, gastric cancer, colorectal cancer, breast cancer, prostate cancer, endometrial cancer, sarcoma, pheochromocytoma adrenal tumor, testicular germ cell tumor, cervical cancer, carcinoma of sexual and reproductive organs, Hodgkin's disease, esophageal cancer, small intestinal cancer, endocrine cancer, thyroid cancer, parathyroid cancer, adrenal cancer, soft tissue sarcoma, bladder cancer, kidney cancer, renal cell carcinoma, renal pelvic carcinoma, central nervous system (CNS) neoplasm, neuroectodermal tumor, spinal tumor, glioma, meningioma or pituitary adenoma, and in other example of the present invention, the pharmaceutical composition induces cancer cell death, or inhibits at least one selected from the group consisting of growth, migration, invasion and metastasis of cancer cells.

In one example of the present invention, it comprises any one nucleic acid sequence of SEQ ID NOs: 7 to 10, and is any one selected from the group consisting of shRNA, siRNA, antisense RNA, antisense DNA, chimeric antisense DNA/RNA, miRNA, and ribozyme, and in one example of the present invention, the nucleic acid molecule complementarily binds to a gene or mRNA encoding a FOXM1 protein, and in other example of the present invention, it specifically binds to the 187th to 207th nucleic acids or the 709th to the 729th nucleic acids of SEQ ID NO: 1.

In one example of the present invention, provided is a recombinant viral vector comprising any one nucleic acid sequence of SEQ ID NOs: 7 to 10, and in another example of the present invention, the recombinant viral vector expresses any one selected from the group consisting of shRNA, siRNA, antisense RNA, antisense DNA, chimeric antisense DNA/RNA, miRNA, and ribozyme.

In one example of the present invention, provided is a method for treating cancer, comprising administering the pharmaceutical composition; nucleic acid molecule; or recombinant viral vector into a subject in need of treating cancer.

### ADVANTAGEOUS EFFECTS

The protein and/or peptide or shRNA comprising the FoxMl point mutation can be usefully used for treatment of various kinds of diseases caused by abnormal growth of cancer cells, especially, cancer diseases such as primary and metastatic solid cancers and leukemia and the like.

### BRIEF DESCRIPTION OF THE DRAWINGS

FIG. 1 shows the results of analyzing the correlation between the expression of FoxM1 and PLK1 and the survival rate in patients with adenocarcinoma patients among non-small cell lung cancer patients.

A: This is the graph analyzing the correlation between the mRNA expression of FoxM1 and PLK1 in lung cancer through Spearman and Pearson using big data in cBioPortal.

B: This is the result of analyzing the correlation between PLK1 and FoxM1 in the mRNA expression of NSCLC patients by cBio-Portal Spearman's coefficient.

C: This is the result of analyzing the correlation between PLK1 and FoxM1 in the mRNA expression of NSCLC patients by cBio-Portal Pearson's coefficient.

D: This is the graph analyzing the survival rate of lung cancer patients according to the expression of PLK1 and FoxM1 by KM PLOTTER analysis.

E: This is the graph analyzing the survival rate of lung cancer patients with metastatic cancer according to the expression of PLK1 and FoxM1 by KM PLOTTER analysis.

F: This is the graph analyzing the survival rate of lung cancer patients according to metastatic lung cancer progression stages, according to the expression of PLK1 and FoxM1 by KM PLOTTER analysis.

G: This is the graph showing the expression of epithelial-mesenchymal markers in lung cancer patients according to metastatic lung cancer progression stages by Heatmap analysis.

FIG. 2 shows the results of analyzing the clinical relevance between FoxM1 and the acti ve form of PLK1 in metastatic lung cancer cells.

A: This is the graph showing the mRNA expression of FoxM1, PLK1, and epithelial-mesenchymal markers in the A549 lung cancer cells treated with TGF-β by real-time polymerase chain reaction (Real-time PCR).

B: This is the graph showing the mRNA expression of FoxM1, PLK1, and epithelial-mesenchymal markers in the H358 lung cancer cells treated with TGF-β by real-time polymerase chain reaction (Real-time PCR).

C: This is the graph showing the mRNA expression of FoxM1, PLK1, and epithelial-mesenchymal markers in the H460 lung cancer cells treated with TGF-β by real-time polymerase chain reaction (Real-time PCR).

D: This is the result of observing the protein expression of FoxM1, PLK1, and epithelial-mesenchymal markers in the lung cancer cells A549, H358, and H460 treated with TGF-β by immunoblotting.

E: This is the result of observing a decrease in phosphorylation of FoxM1 and PLK1 by treatment with phosphatase (CIP) after treating the A549 cells with 5 ng/ml of TGF-β.

F: This is the result of observing a decrease in phosphorylation of FoxM1 and PLK1 by treatment with phosphatase (CIP) after treating the H460 cells with 5 ng/ml of TGF-β.

FIG. 3 shows the results of analyzing phosphorylation and the phosphorylation sites of FoxM1 by the active form of PLK1.

A: This is the result of observing the interaction between FoxM1 and PLK1 by immunoprecipitation using a Myc antibody against Myc-labeled FoxM1.

B: This is the result of observing the interaction between FoxM1 and PLK1 by performing immunoprecipitation using PLK1 antibody under the conditions of treating A549 cells with 5 ng/ml of TGF-β.

C: This is the result of observing the interaction between FoxM1 and PLK1 by performing immunoprecipitation using PLK1 antibody under the conditions of treating H460 cells with 5 ng/ml of TGF-β.

D: This is the result of observing that the active form of PLK1 phosphorylates FoxM1 by performing a phosphorylase reaction method using GST-labeled FoxM1 with the active form of PLK1 (PLK1 TD).

E: This is the result of determining FoxM1 phosphorylatable candidate sites phosphorylated by PLK1 through liquid chromatography mass spectrometry.

F: This is the result of performing a kinase assay using the active form of PLK1 and the non-phosphorylated point mutant protein in which the phosphorylation candidate sites of FoxM1 were substituted with alanine by site-directed mutagenesis.

G: This is the result of performing a kinase assay using the active form of PLK1 and the non-phosphorylated point mutant protein in which the phosphorylation candidate sites of FoxM1 were substituted with alanine.

H: This is the result showing the FoxM1 phosphorylation sites in various species.

FIG. 4 shows the experiment assessing the effects of the over-expression of phosphorylation and non-phosphorylated point mutants of FoxM1 on the motility and invasion of cancer cells in A549 lung cancer cells.

A: This is the result of observing the presence of over-expression of FoxM1 protein and the changes in the mesenchymal transition marker (N-cadherin) by immunoblotting, after FoxM1 phosphorylation and non-phosphorylated point mutants were expressed in A549 lung cancer cells.

B: This is the result of observing the presence of over-expression of FoxM1 mRNA and the changes in the mRNA of the mesenchymal transition marker (N-cadherin) by chain reaction (Real-time PCR), after FoxM1 phosphorylation and non-phosphorylated point mutants were expressed in A549 lung cancer cells.

C: This is the result of observing the degree of cell proliferation of cells expressing each point mutant over time after FoxM1 phosphorylation and non-phosphorylated point mutants were expressed in A549 lung cancer cells.

D: This is the result of observing cell motility by a microscope through a migration assay using an insert in the A549 lung cancer cells expressing FoxM1 phosphorylation and non-phosphorylated point mutants.

E: This is the result of observing the invasion of cells through an invasion assay using Matrigel and inserts in the A549 lung cancer cells expressing FoxM1 phosphorylation and non-phosphorylated point mutants.

F: This is the result of observing cell motility through a wound healing assay in the A549 lung cancer cells expressing FoxM1 phosphorylated point mutant and a simultaneous triple-point mutant.

G: The relative healing distance at 72 h through a wound healing assay with the A549 lung cancer cells expressing FoxM1 phosphorylated point mutant and simultaneous triple-point mutant is displayed in a graph.

FIG. 5 shows the experiment assessing the metastasis and tumorigenic potential of cancer cells over-expressing phosphorylation and non-phosphorylated point mutants of FoxM1 in animal models.

A: This is the result of observing the metastatic tumor formation and suppression effects in the lungs of mice when lung cancer cells expressing FoxM1 phosphorylation and non-phosphorylated point mutants were injected intravenously.

B: This is the result of observing the tumor formation and suppression effects in each experimental group through H&E staining of cancer tissues produced by metastasis to the lungs of mice when lung cancer cells expressing FoxM1 phosphorylation and non-phosphorylated point mutants were injected intravenously.

C: This is the result of observing the tumor formation and suppression effects in each experimental group through Ki-67 staining of cancer tissues produced by metastasis to the lungs of mice when lung cancer cells expressing FoxM1 phosphorylation and non-phosphorylated point mutants were injected intravenously.

D: This is the result of observing changes in epithelial-mesenchymal protein markers and immune evasion factor proteins in the homogenized lung tissues obtained from the mice when lung cancer cells expressing FoxM1 phosphorylation and non-phosphorylated point mutants were injected intravenously.

E: This is the graph showing the mRNA expression of epithelial-mesenchymal transition markers and immune evasion factors using real-time polymerase chain reaction (Real-time PCR) in the homogenized lung tissues obtained from the mice when lung cancer cells expressing FoxM1 phosphorylation and non-phosphorylated point mutants were injected intravenously.

F: This is the result of the apoptotic degree in lung cancer cells expressing FoxM1 phosphorylation and non-phosphorylated point mutants by measuring the activity of the caspase-3 enzyme.

FIG. 6 shows the results of observing the effects on polarization into tumor-associated macrophages (TAM) from monocyte THP-1 cells co-cultured with lung cancer cells expressing point mutants of FoxM1 protein.

A: This is the graph showing the mRNA expression of M1 and M2 markers in THP-1 cells by real-time polymerase chain reaction (Real-time PCR) after co-culture of lung cancer cells expressing point mutants of FoxM1 protein and monocyte THP-1 cells.

B: This is the graph showing the mRNA expression of M2-inducing factors and immune evasion factors in A549 cells by real-time polymerase chain reaction (Real-time PCR) after co-culture of lung cancer cells expressing point mutants of FoxM1 protein and monocyte THP-1 cells.

C: This is the result of observing the expression of TGFB1 and VEGFA in the culture medium expressing the point mutants of FoxM1 protein through an ELISA assay.

D: This is the graph showing the mRNA expression of M2 markers in THP-1 cells by real-time polymerase chain reaction (Real-time PCR) after co-culture of mutant cancer cells in which FoxM1 was knocked down and THP-1 cells.

E: This is the result of observing the staining of CD68 and CD163, which are markers for TAM, with lung tissues produced by metastasis to the lungs of mice when lung cancer cells expressing FoxM1 phosphorylation and non-phosphorylated point mutants were injected intravenously.

F: This is the graph quantifying the staining results of CD68 and CD163, which are markers for TAM, with lung tissues produced by metastasis to the lungs of mice when lung cancer cells expressing FoxM1 phosphorylation and non-phosphorylated point mutants were injected intravenously.

G: This is the result of observing changes in CD68 and CD163 proteins, which are markers for TAM, with lung tissues produced by metastasis to the lungs of mice when lung cancer cells expressing FoxM1 phosphorylation and non-phosphorylated point mutants were injected intravenously.

FIG. 7 shows the result of observing the effects of cancer cells expressing point mutants of the FoxM1 protein on cancer cell immune evasion through polarization of the co-cultured monocyte THP-1 cells and Jurkat cells, the T cells.

A: This is the graph showing the viability of lung cancer cells when co-culturing lung cancer cells expressing point mutants of FoxM1 protein with monocyte THP-1 cells at a certain ratio.

B: This is the graph showing the mRNA expression of immune evasion factors in THP-1 cells by real-time polymerase chain reaction (Real-time PCR) when co-culturing lung cancer cells expressing point mutants of FoxM1 protein with monocyte THP-1 cells.

C: This is the graph showing the mRNA expression of immune evasion factors in lung cancer cells expressing point mutants of FoxM1 by real-time polymerase chain reaction (Real-time PCR).

D: This is the graph showing the viability of lung cancer cells when co-culturing lung cancer cells expressing point mutants of FoxM1 protein with Jurkat cells, T cells, at a certain ratio.

E: This is the graph showing the mRNA expression of regulatory T cell markers in Jurkat cells by real-time polymerase chain reaction (Real-time PCR) when co-culturing lung cancer cells expressing point mutants of FoxM1 protein with Jurkat cells, T cells, at a certain ratio.

F: This is the graph showing the mRNA expression of immune evasion factors and M2-inducing factors in lung cancer cells expressing point mutants of FoxM1 protein by real-time polymerase chain reaction (Real-time PCR).

G: This is the graph showing the viability of lung cancer cells when co-culturing lung cancer cells expressing point mutants of FoxM1 protein and monocyte THP-1 cells with Jurkat cells, T cells, at a certain ratio.

FIG. 8 shows the results of observing peptide efficacy evaluation by producing FoxM1 non-phosphorylated point mutant peptides and evaluating the apoptosis and metastasis in lung cancer cells.

A: This is the peptide sequence of FoxM1 non-phosphorylated point mutant.

B: This is the result of observing the apoptotic efficacy of 3 types of FoxM1 non-phosphorylated point mutant peptides in A549 cells.

C: This is the result of observing the suppressive efficacy on metastasis of cancer cells by treatment with 3 types of FoxM1 non-phosphorylated point mutant peptides (5 μM FoxM1-S25A peptide) under the conditions of the phosphorylated point mutant of FoxM1 over-expressed in A549 cells.

FIG. 9 shows the results of analyzing the patient survival in various cancer types according to FoxM1 expression.

A: This is the graph analyzing the patient survival rate according to 12 cancer types according to FoxM1 expression.

B: This is the result of observing the apoptotic efficacy of 3 types of FoxM1 non-phosphorylated point mutant peptides in solid cancer.

FIG. 10 shows the results of observing the effects on metastasis of lung cancer cells, polarization to tumor-associated macrophages, and immune evasion ability of cancer cells by producing FoxM1 non-phosphorylated point mutant peptides.

A: This is the peptide sequence of the FoxM1 non-phosphorylated point mutant linked with FITC (a fluorescent material) at the C-terminus of the peptide to observe cell permeability.

B: This is the result of observing the degree of cell permeability of the FoxM1 non-phosphorylated point mutant peptide in A549 cells.

C: This is the result of observing changes in mRNA of mesenchymal transition markers by treatment with the FoxM1 non-phosphorylated point mutant peptide (5 μM FoxM1-S25A peptide) after A549 cells were treated with 5 ng/ml of TGF-β.

D: This is the result of observing changes in the motility of cancer cells by treatment with the FoxM1 non-phosphorylated point mutant peptide (5 μM FoxM1-S25A peptide) after A549 cells were treated with 5 ng/ml of TGF-β.

E: This is the result of observing changes in mRNA of mesenchymal transition markers by treatment with the FoxM1 non-phosphorylated point mutant peptide (5 μM FoxM1-S25A peptide) under the conditions of the phosphorylated point mutant of FoxM1 over-expressed in A549 cells.

F: This is the result of observing changes in the motility of cancer cells by treatment with the FoxM1 non-phosphorylated point mutant peptide (5 μM FoxM1-S25A peptide) under the conditions of the phosphorylated point mutant of FoxM1 over-expressed in A549 cells.

G: This is the result of observing changes in mRNA of STAT1, VEGFA, c-fos, IL6, and CD274 by treatment with the FoxM1 non-phosphorylated point mutant peptide (5 μM FoxM1-S25A peptide) under the conditions of the phosphorylated point mutant of FoxM1 over-expressed in A549 cells.

FIG. 11 shows the results of analyzing the clinical relevance of FoxM1 and the active form of PLK1 in metastatic lung cancer cells.

A: This is the graph analyzing the relevant pathways of invasive cells expressing the active form of PLK1 under the cancer metastatic conditions of A549 lung cancer cells by KEGG 2019 pathway analysis.

B: This is the result of observing the protein expression of FoxM1 and epithelial-mesenchymal transition markers by immunoblotting in A549 lung cancer cells treated with TGF-β.

C: This is the result of observing the protein expression of FoxM1 and epithelial-mesenchymal transition markers by immunoblotting in H358 lung cancer cells treated with TGF-β.

D: This is the result of observing the protein expression of FoxM1 and epithelial-mesenchymal transition markers by immunoblotting in H460 lung cancer cells treated with TGF-β.

FIG. 12 shows the experiment assessing the effects of over-expression of FoxM1 WT by the active form of PLK1 on the motility and invasion of cancer cells.

A: This is the result of observing the presence of FoxM1 protein overexpression and changes in EMT markers by immunoblotting after FoxM1 WT was expressed in A549 lung cancer cells.

B: This is the result of observing the presence of FoxM1 protein overexpression and the changes in EMT markers by real-time polymerase chain reaction (Real-time PCR) after FoxM1 WT was expressed in A549 lung cancer cells.

C: This is the graph showing the motility of cancer cells by a migration assay using an insert in the A549 lung cancer cells expressing FoxM1 WT using an Odyssey infrared imaging system to analyze the intensity of cancer cells stained with crystal violet, which indicates the cell motility.

D: This is the result of observing the invasion of cells by an invasion assay in the A549 lung cancer cells expressing FoxM1 WT using Matrigel and an insert.

E: This is the result of observing the expression of CD274, which is an immune evasion factor, in the A549 lung cancer cells expressing FoxM1 WT using real-time polymerase chain reaction (Real-time PCR).

FIG. 13 shows the results of observing an increase in TAM markers and the polarization of THP-1 cells when co-culturing FoxM1 WT-expressed cancer cells and human monocyte THP-1 cells.

A: This is the result of observing the expression of M1 and M2 markers in THP-1 cells after co-culturing FoxM1 WT-expressed cancer cells and human monocyte THP-1 cells.

B: This is the result of observing the expression of M2-inducing factors in A549 cells after co-culturing FoxM1 WT-expressed cancer cells and human monocyte THP-1 cells.

C: This is the result of observing the expression of TGFB1 and VEGFA in the culture medium where the cancer cells expressing FoxM1 WT were cultured for 48 hours by ELISA assay.

D: This is the result of observing CD68, which is a macrophage marker, and CD163, which is a tumor-associated macrophage marker, by immunostaining in the animal lung tissues injected with cancer cells expressing FoxM1 WT.

FIG. 14 is a result of observing suppressive effects on FoxM1 expression when treated with each FoxM1 shRNA to inhibit FoxM1 mRNA expression in lung cancer cells.

A: This is the graph showing the degree of suppression in FoxM1 expression as the mRNA expression pattern by treatment with each FoxM1 shRNA in A549 lung cancer cells.

B: This is the result showing the degree of suppression in FoxM1 expression as the protein expression pattern by treatment with each FoxM1 shRNA in A549 lung cancer cells.

C: This is the result showing the inhibitory effects on cancer cell motility of suppression of FoxM1 expression at 24-hour intervals by treatment with the FoxMl shRNA in A549 lung cancer cells.

D: This is the result showing the inhibitory effects on cancer cell motility of suppression of FoxM1 expression at the 72-hour time point by treatment with the FoxM1 shRNA in A549 lung cancer cells.

E: This is the graph showing the mRNA expression pattern of epithelial-mesenchymal markers (CDH1 and CDH2) affected by suppression of FoxM1 expression by treatment of A549 lung cancer cells with FoxM1 shRNA under the cancer metastatic conditions induced by TGF-β treatment.

F: This is the graph showing the induction of cancer cell apoptosis by treating A549 lung cancer cells with FoxM1 shRNA.

FIG. 15 shows the results of observing the polarization potential of macrophages when the A549 lung cancer cells expressing FoxM1 WT protein were treated with FoxM1 shRNA, an inhibitor for FoxM1 mRNA expression.

A: This is the result showing the mRNA expression of FoxM1 when the A549 lung cancer cells expressing FoxM1 WT protein were treated with FoxM1 shRNA, an inhibitor for FoxM1 mRNA expression.

B: This is the result of observing changes in TAM markers in THP-1 when co-culturing FoxM1 shRNA-treated cancer cells with human monocyte THP-1 cells.

C: This is the result showing the mRNA expression of IFITM1 when the A549 lung cancer cells expressing FoxM1 WT protein were treated with FoxM1 shRNA, an inhibitor for FoxM1 mRNA expression.

FIG. 16 is a result of observing changes in epithelial-mesenchymal transition markers and M2-inducing factors when lung cancer cells were treated with Thiostrepton, a FoxM1 inhibitor.

A: This is the result showing the mRNA expression pattern of mesenchymal transition markers (CDH2, VIM, SNAI1, and SNAI2) when inhibiting FoxM1 function by treating A549 lung cancer cells with Thiostrepton in the cancer cell metastatic environment induced by over-expression of FoxM1 WT.

B: This is the result showing the mRNA expression pattern of M2-inducing factors (IL6 and VEGFA) and an immune evasion factor (CD274) when inhibiting FoxM1 function by treatment A549 lung cancer cells with Thiostrepton in the cancer cell metastatic environment induced by over-expression of FoxM1 WT.

FIGS. 17 to 19 show the nucleic acid sequence of FOXM1 WT gene (SEQ ID NO: 3) and the amino acid sequence of FoxM1 protein (SEQ ID NO: 1), FIGS. 20 to 22 show the sequences of SEQ ID Nos: 1 to 11: SEQ ID NO: 6 is human FOXM1B mRNA of accession number U74613. SEQ ID NO: 7 is the forward sequence of the first target according to an example of the present invention. SEQ ID NO: 8 is the reverse sequence of the first target according to an example of the present invention. SEQ ID NO: 9 is the forward sequence of the second target according to an example of the present invention. SEQ ID NO: 10 is the reverse sequence of the second target according to an example of the present invention. SEQ ID NO: 11 is the protein sequence of FOXM1 1B.

### MODE FOR INVENTION

Hereinafter, the present invention will be described in detail by embodiments of the present invention with reference to the attached drawings. However, the following embodiments are provided as examples of the present invention, and if it is judged that a detailed description of a technology or configuration well known to those skilled in the art may unnecessarily obscure the gist of the present invention, the detailed description may be omitted., the present invention is not limited thereby. The present invention is capable of various modifications and applications within the description of the claims described below and the scope of equivalents interpreted therefrom.

In addition, terminologies used in the present specification are terms used to appropriately express preferred embodiments of the present invention, and they may vary depending on the intention of the user or operator or the customs of the field to which the present invention belongs. Thus, definitions of these terminologies should be made based on the contents throughout the present specification. Throughout the specification, when a part is said to “comprise” a certain element, this means that it may further comprise other elements rather than excluding other elements, unless otherwise specifically stated.

All technical terms used in the present invention, unless otherwise defined, are used with the same meaning as commonly understood by those skilled in the art in the field related to the present invention. In addition, in the present specification, preferred methods or samples are described, but those similar or equivalent thereto are also included in the scope of the present invention. The contents of all publications described in the present specification by reference are incorporated to the present invention.

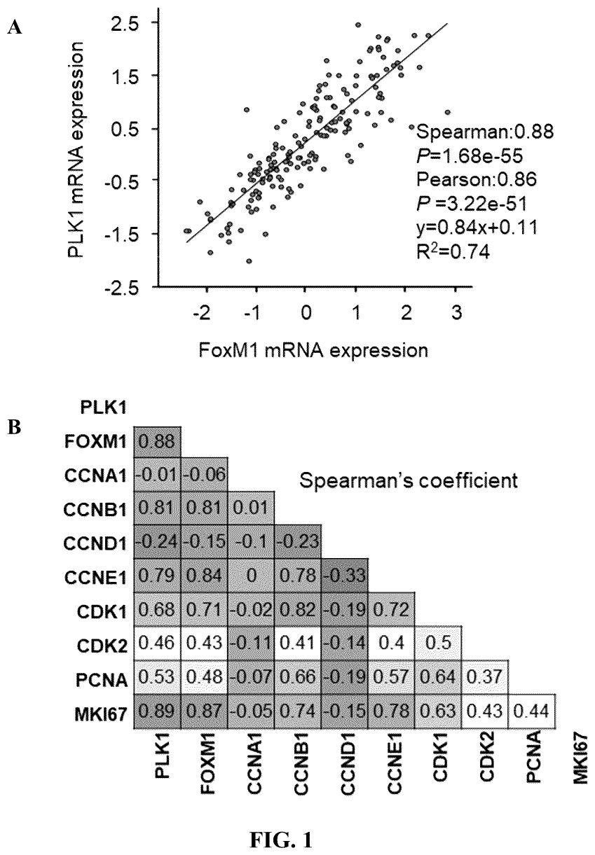

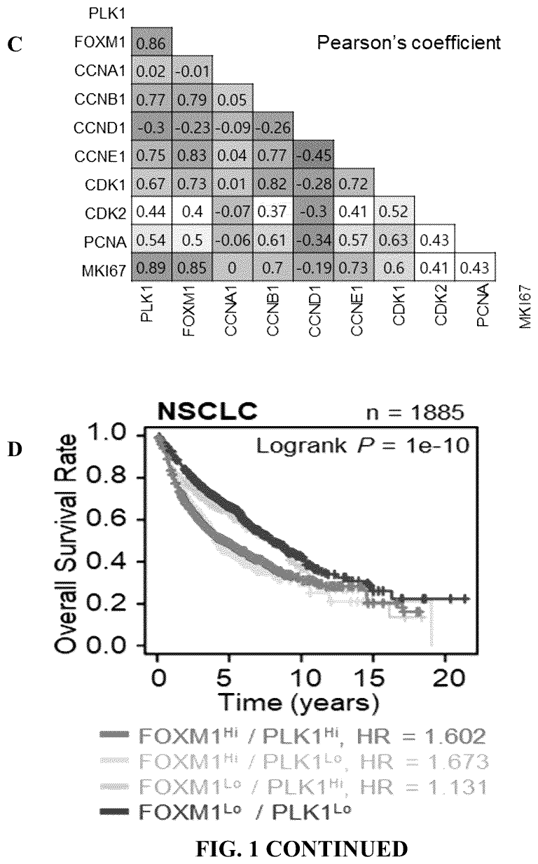

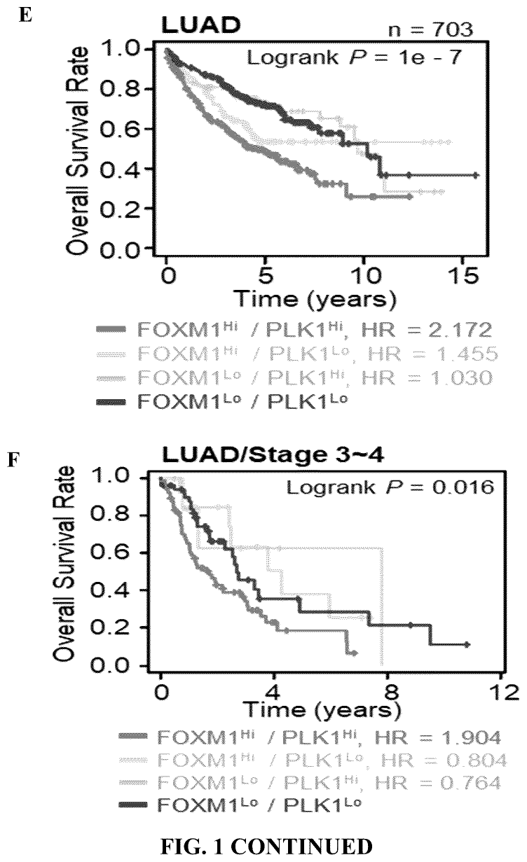

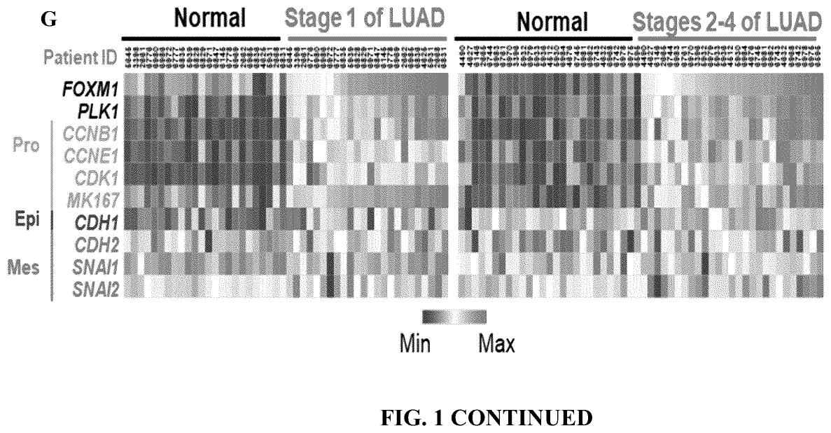

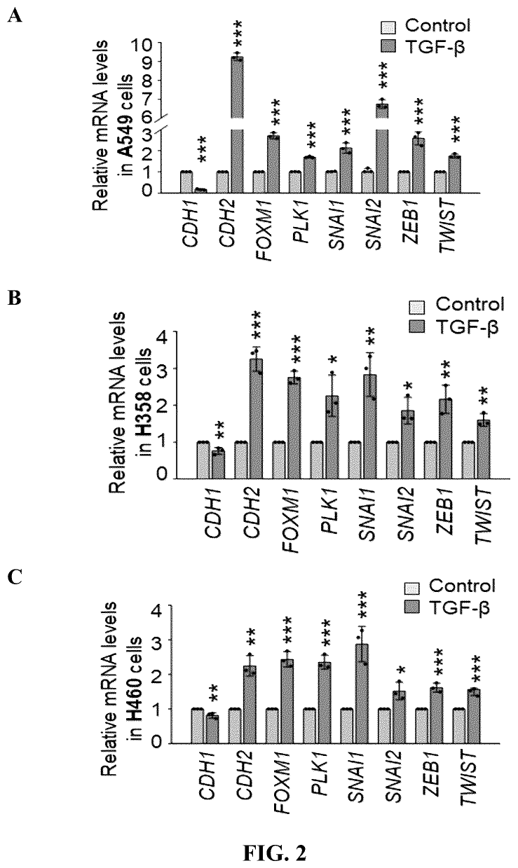

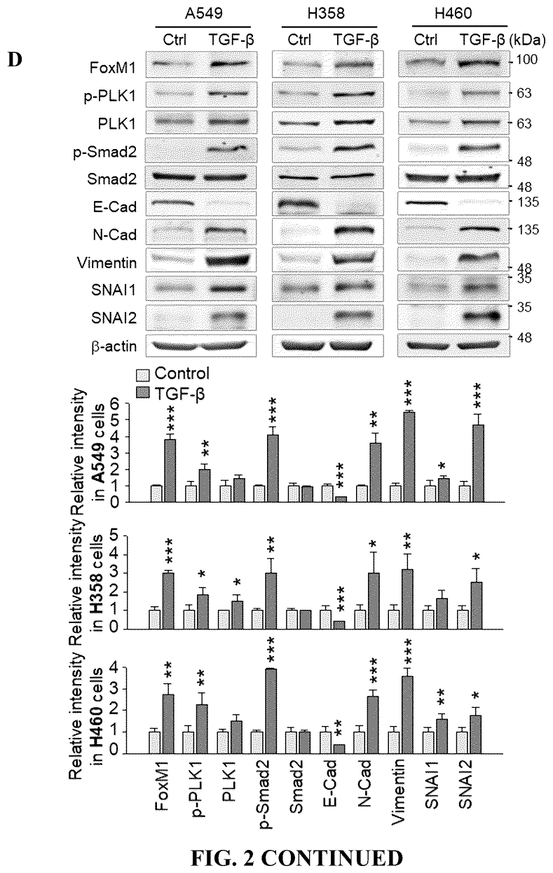

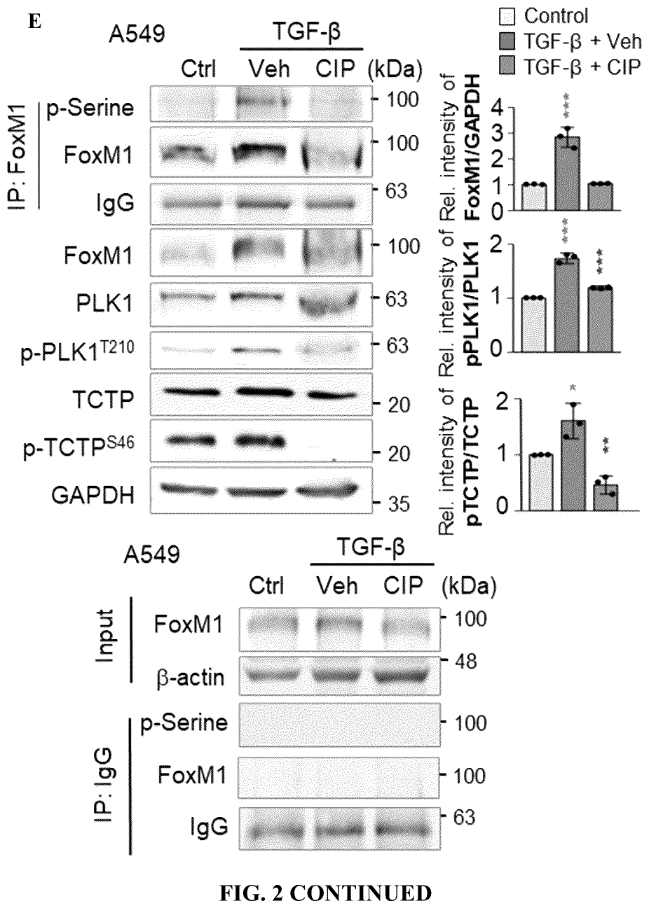

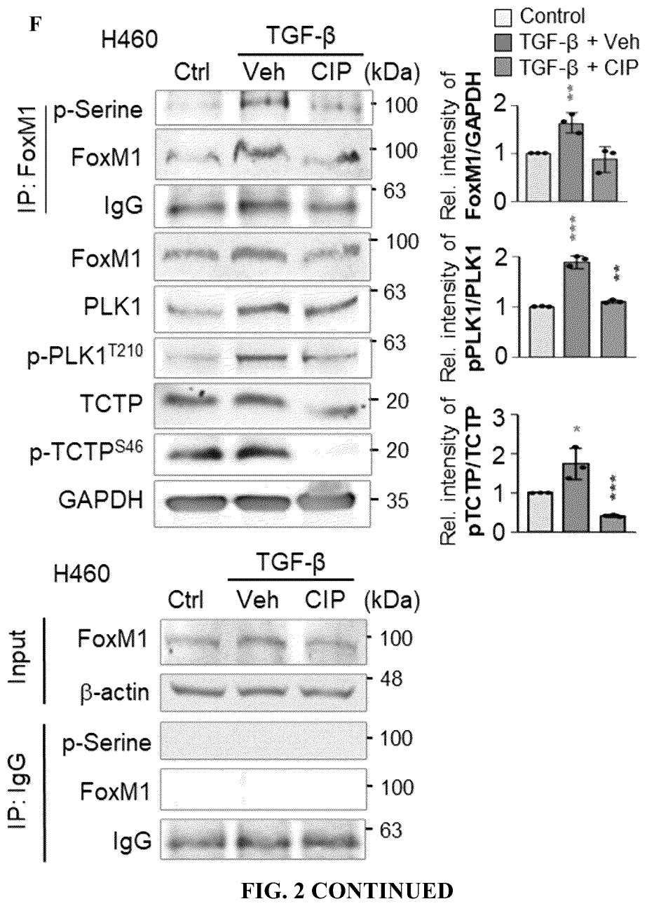

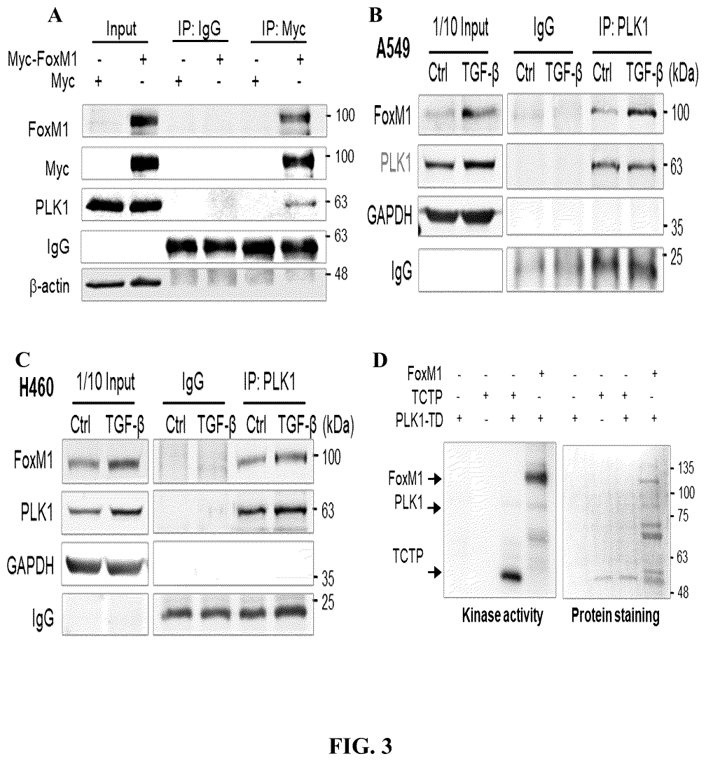

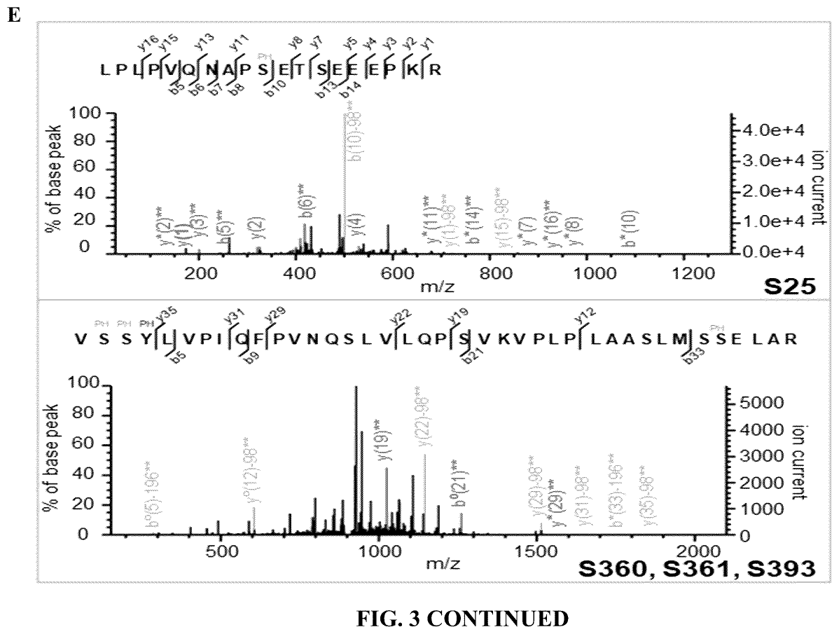

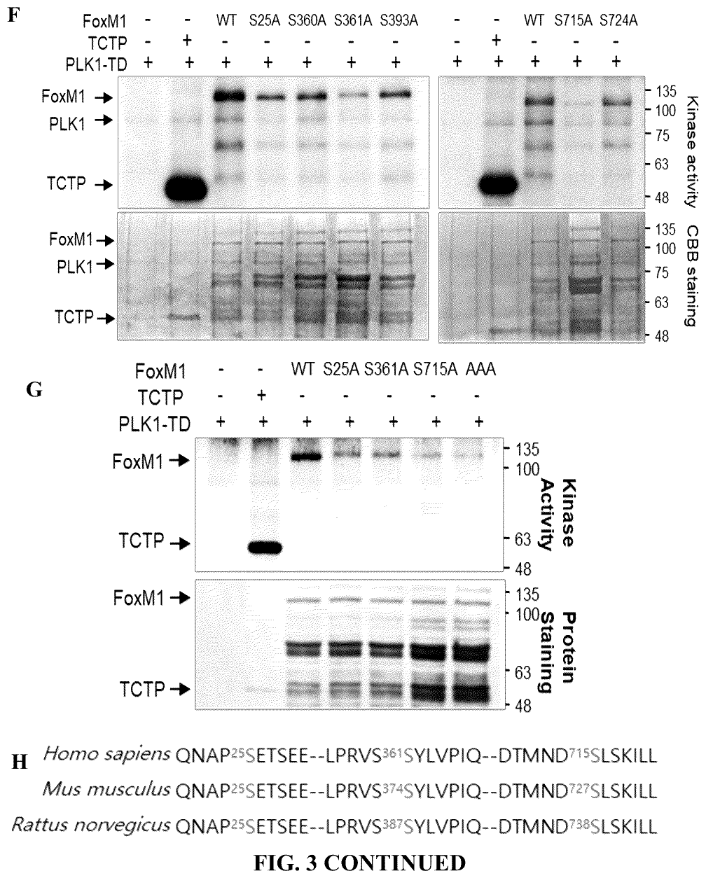

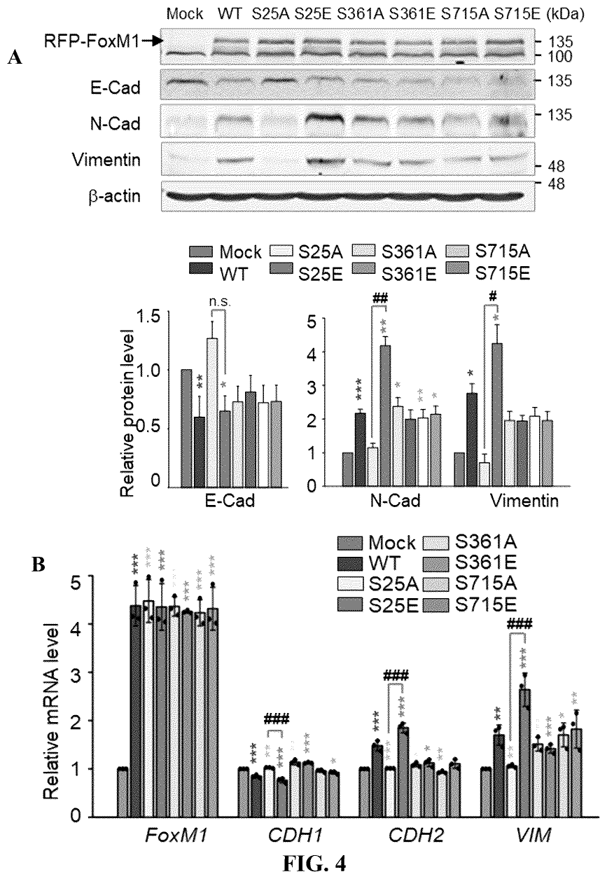

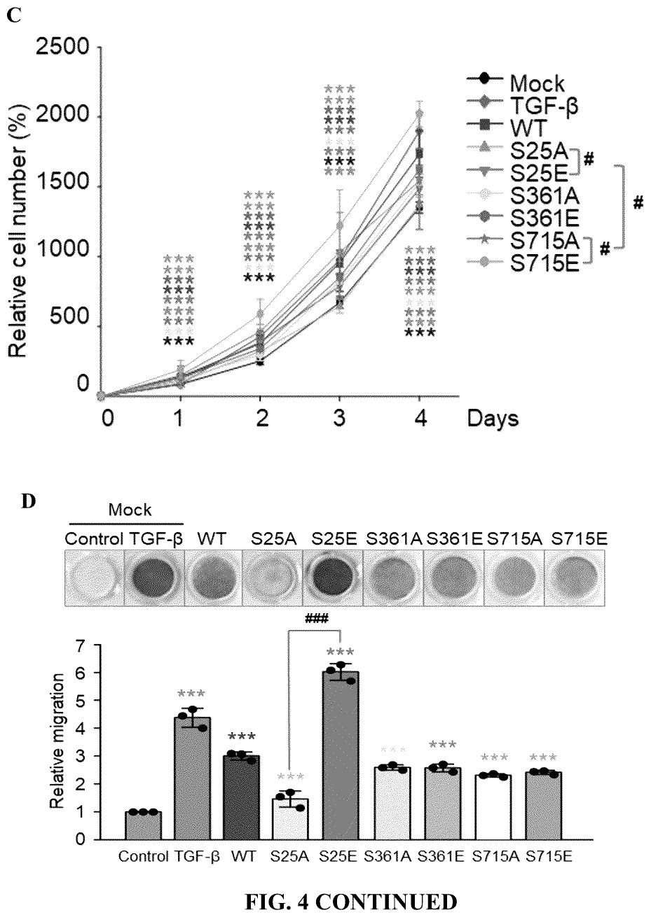

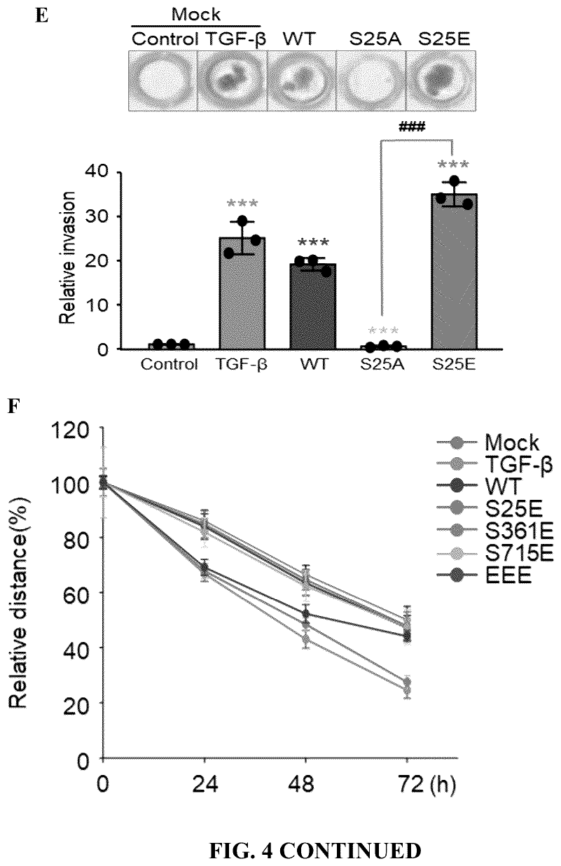

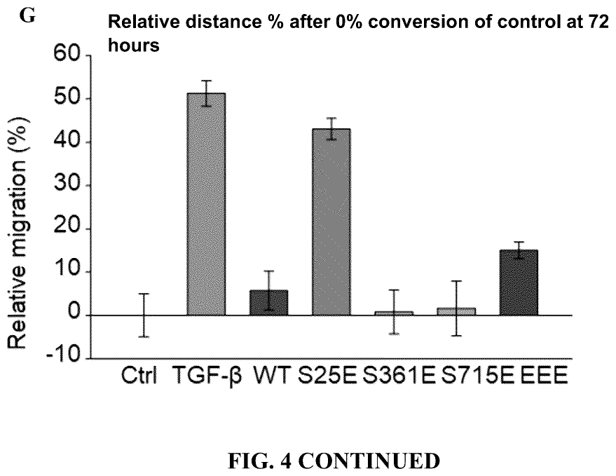

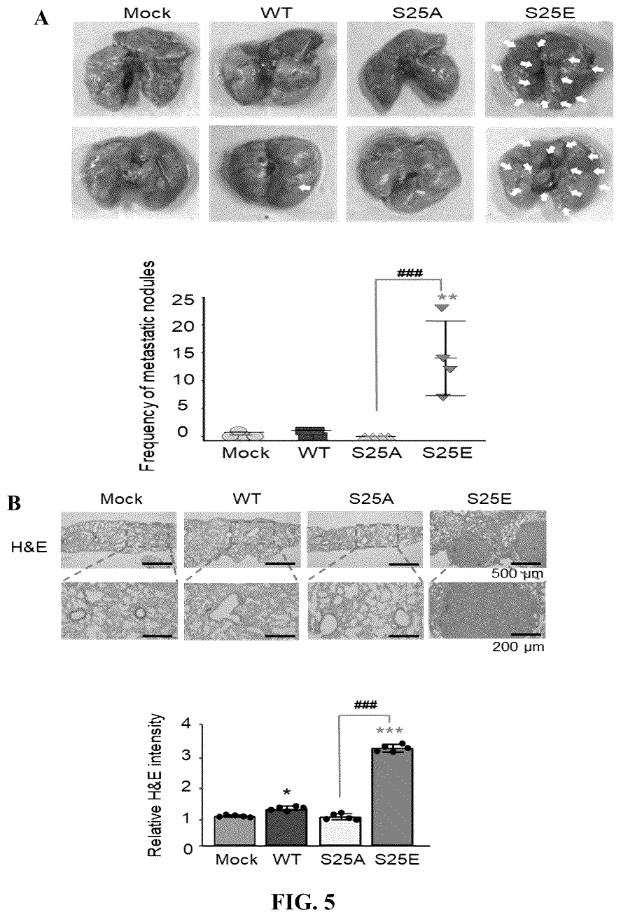

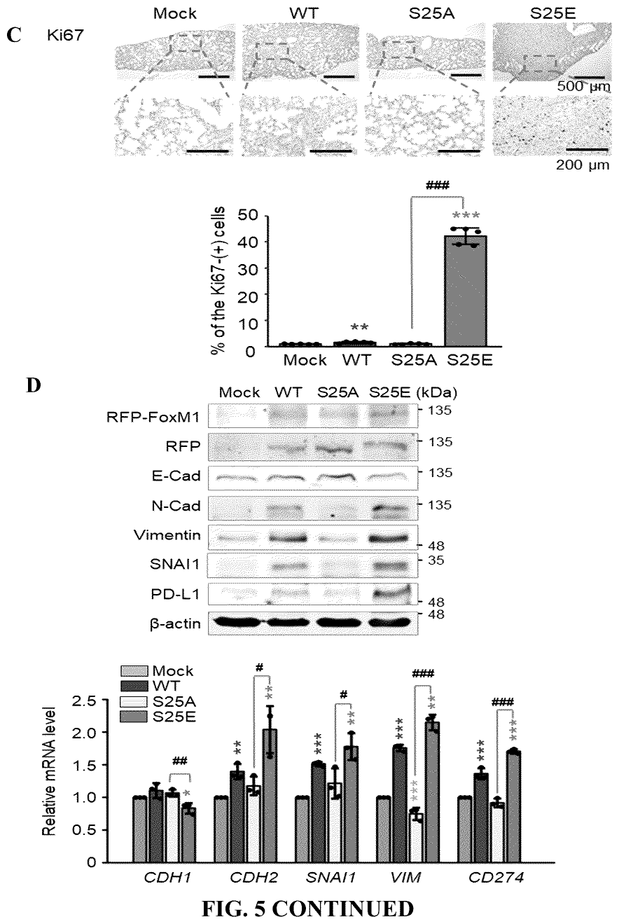

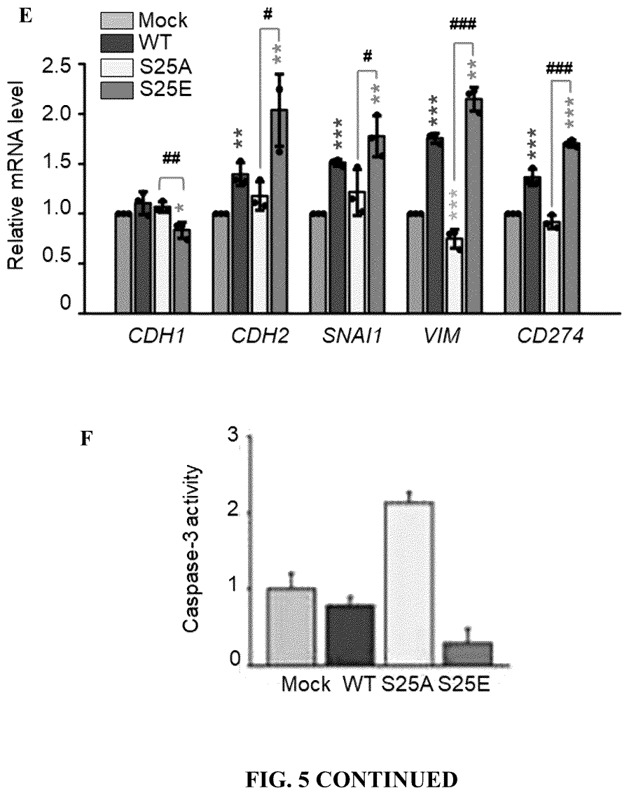

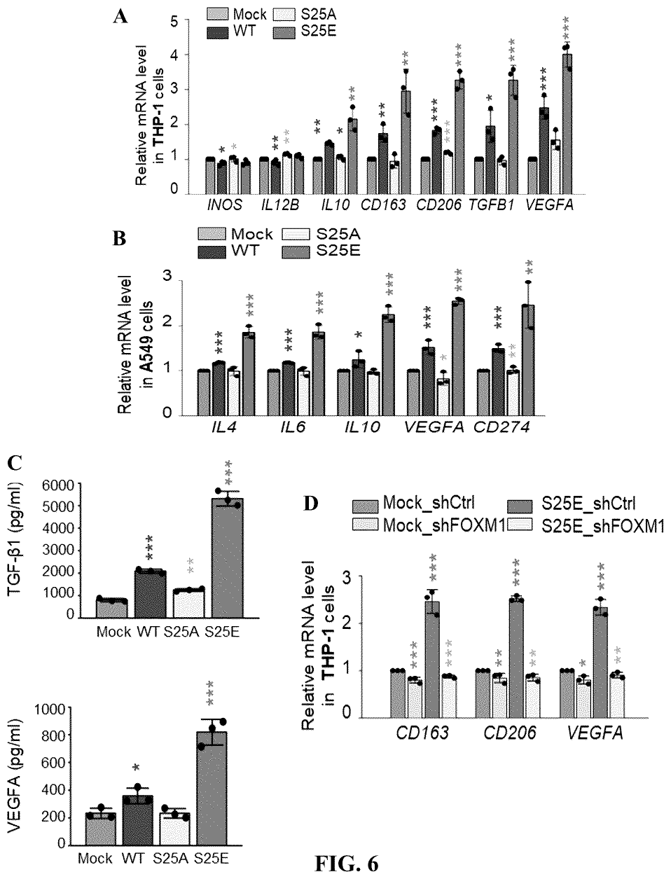

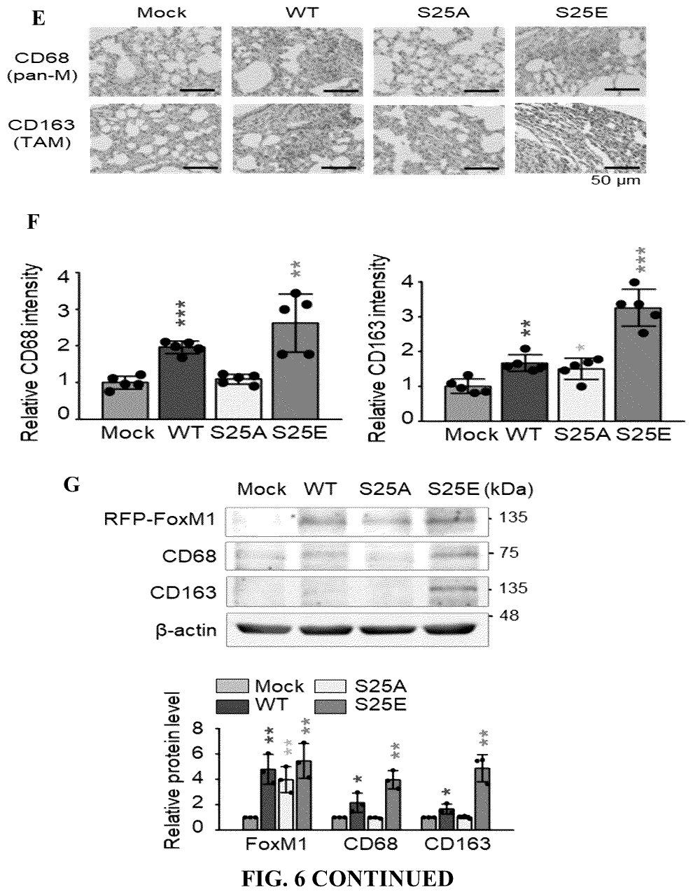

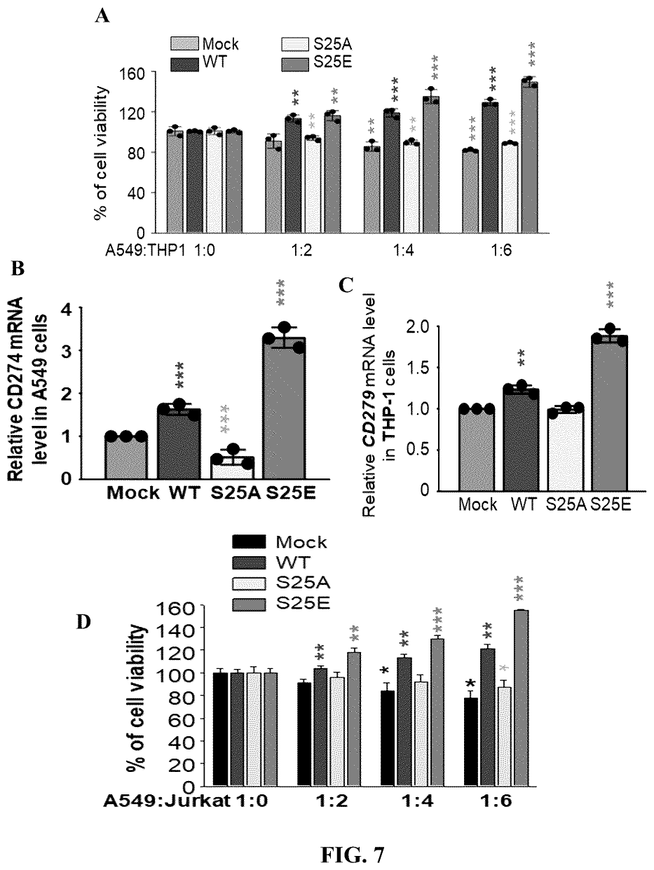

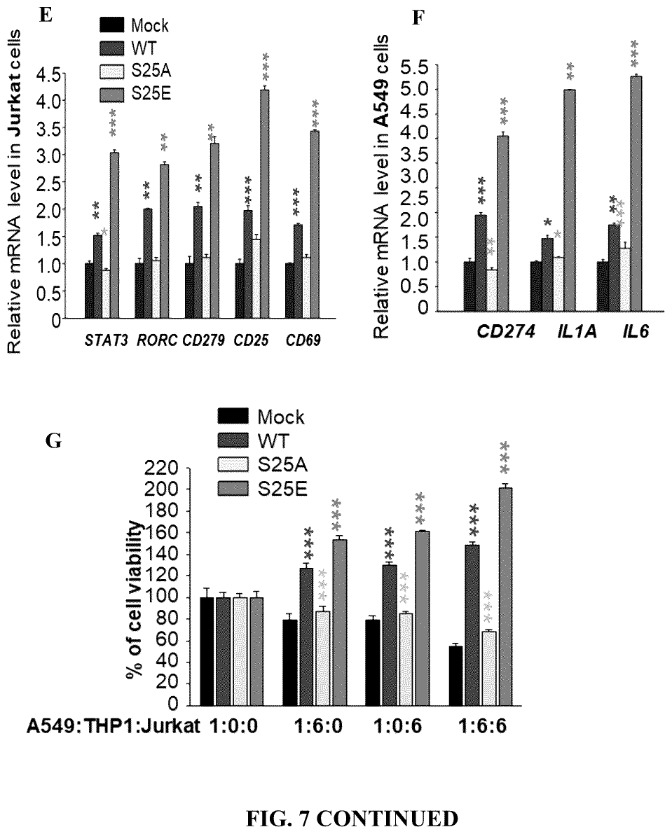

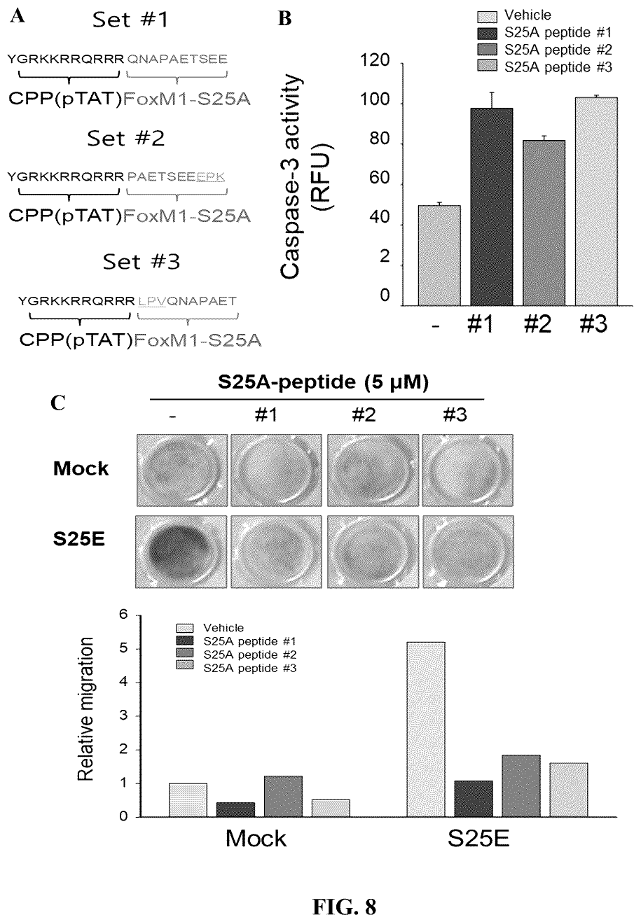

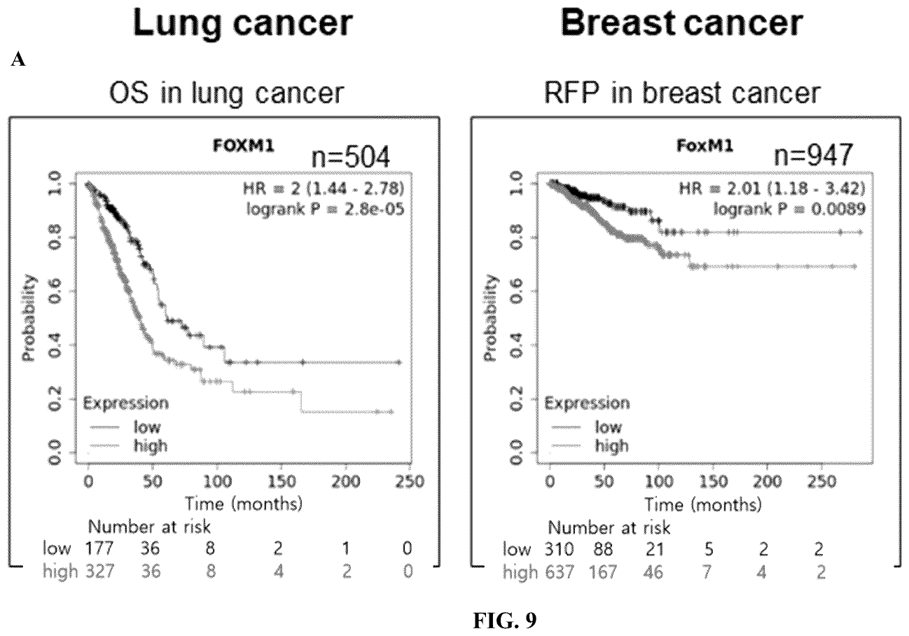

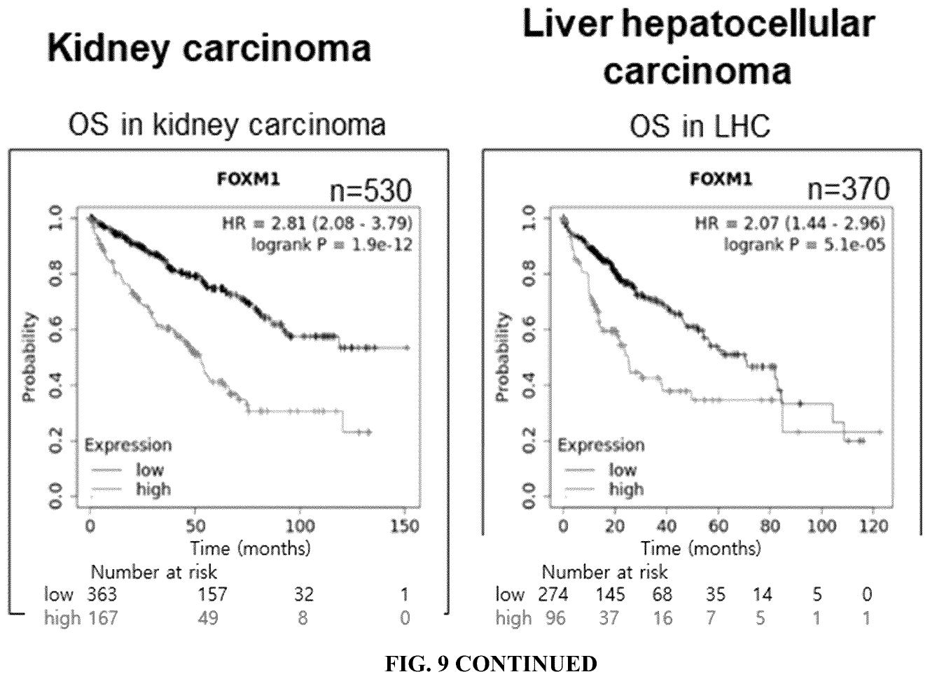

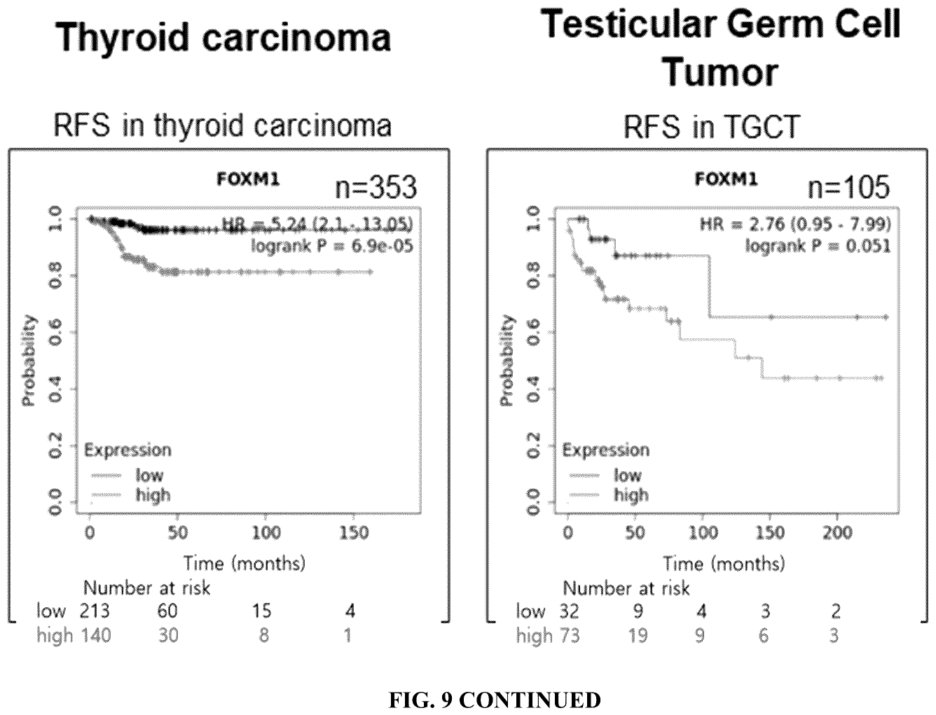

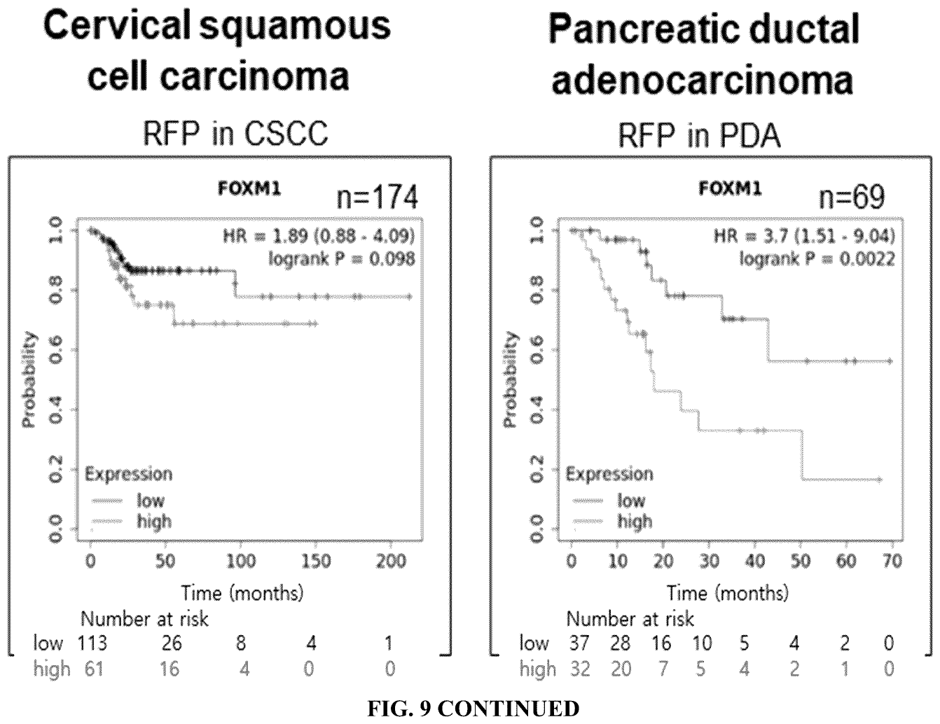

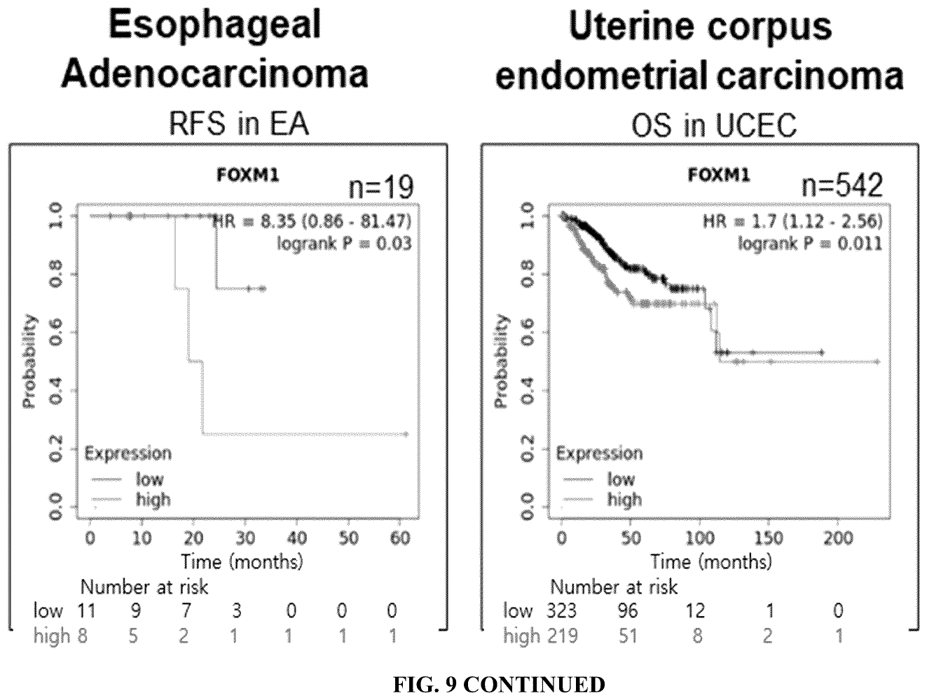

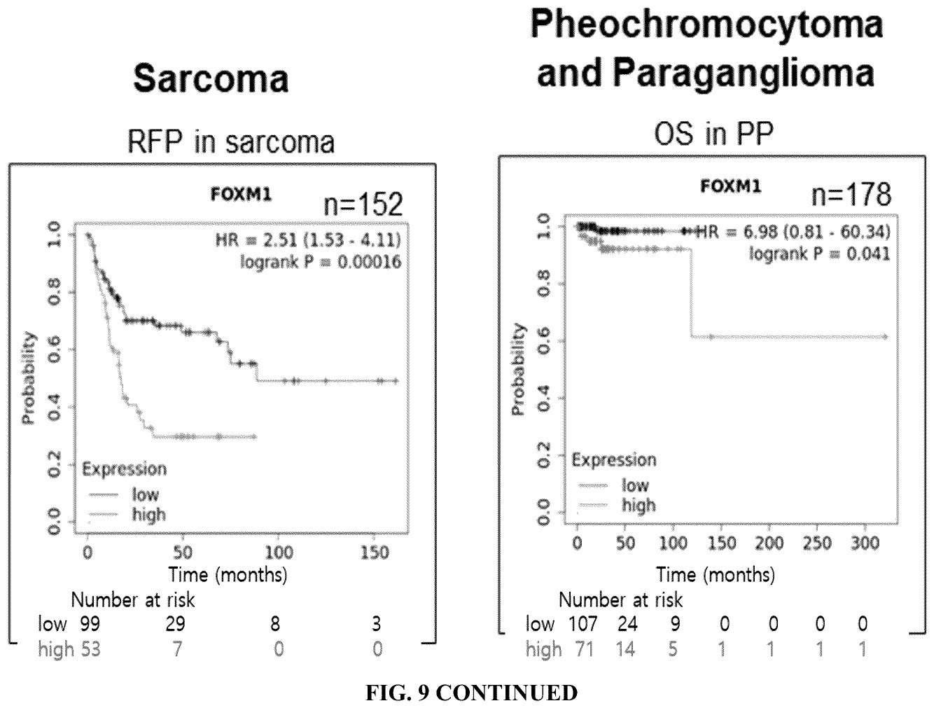

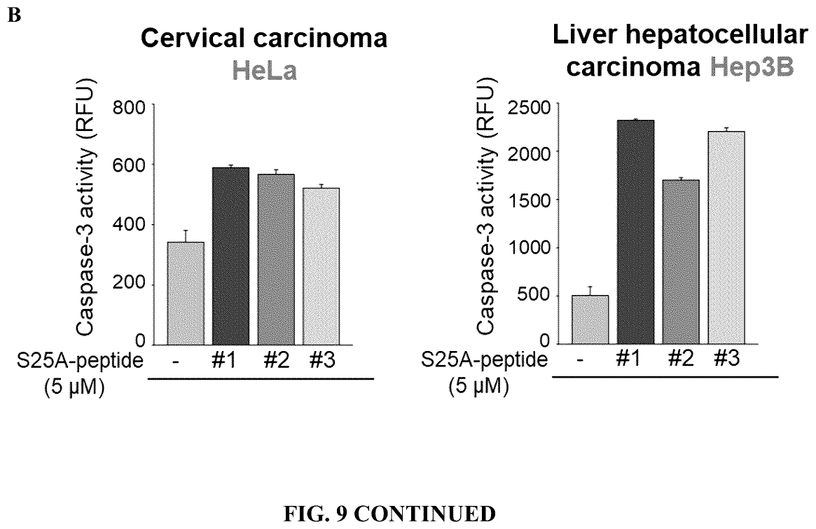

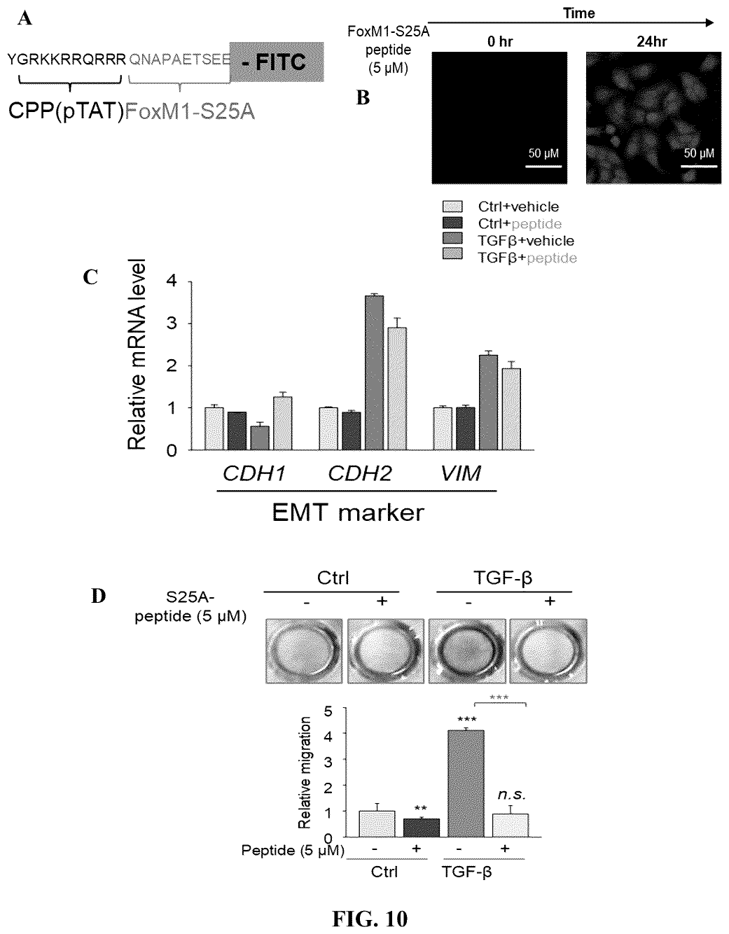

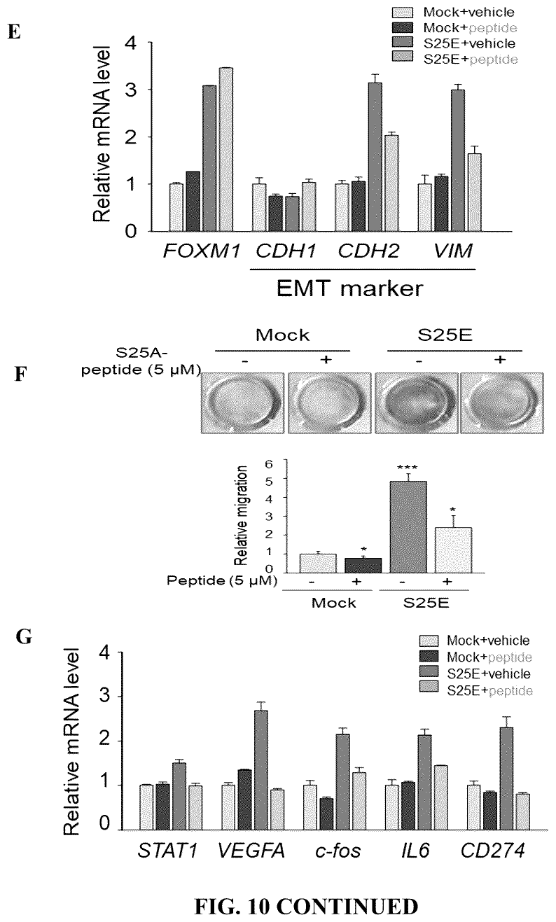

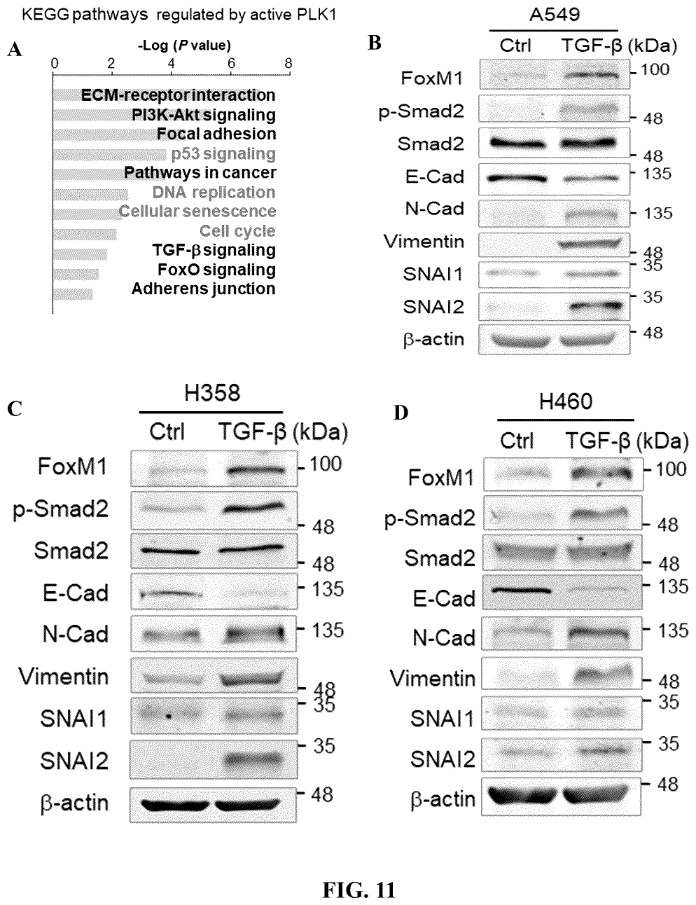

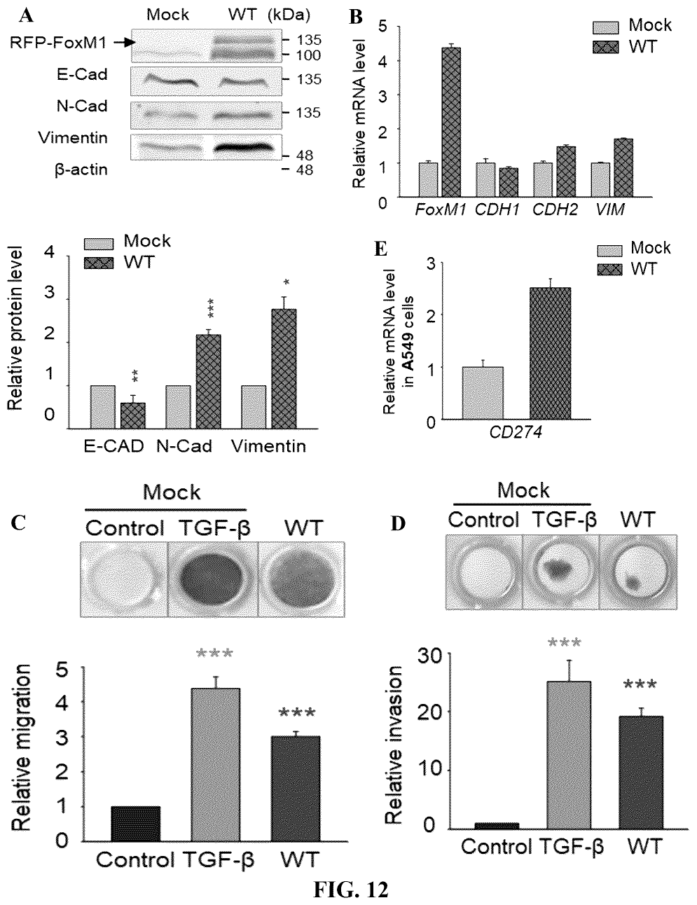

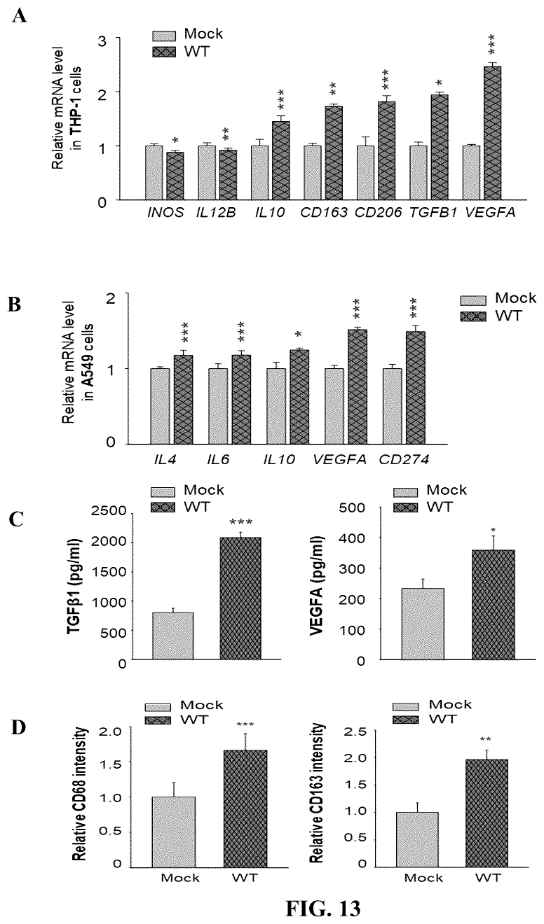

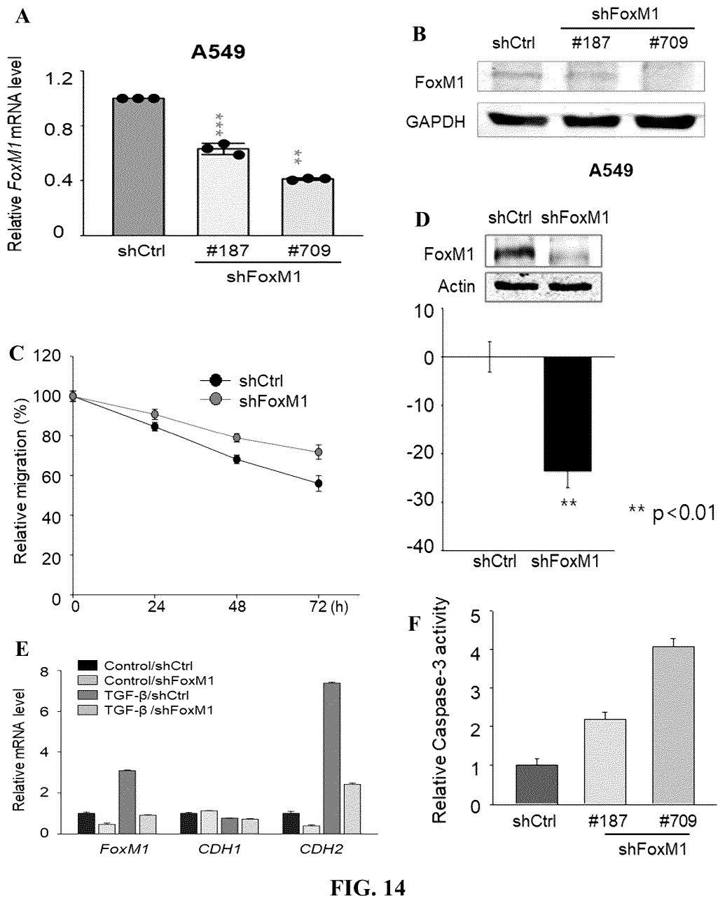

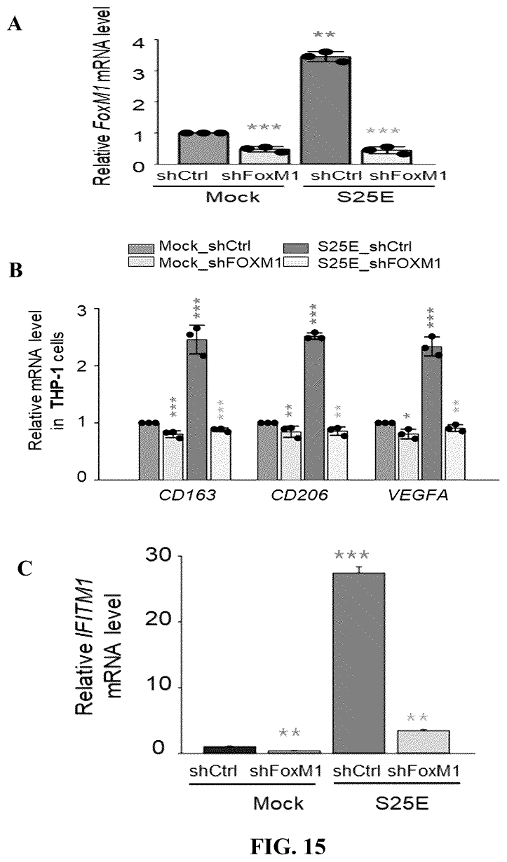

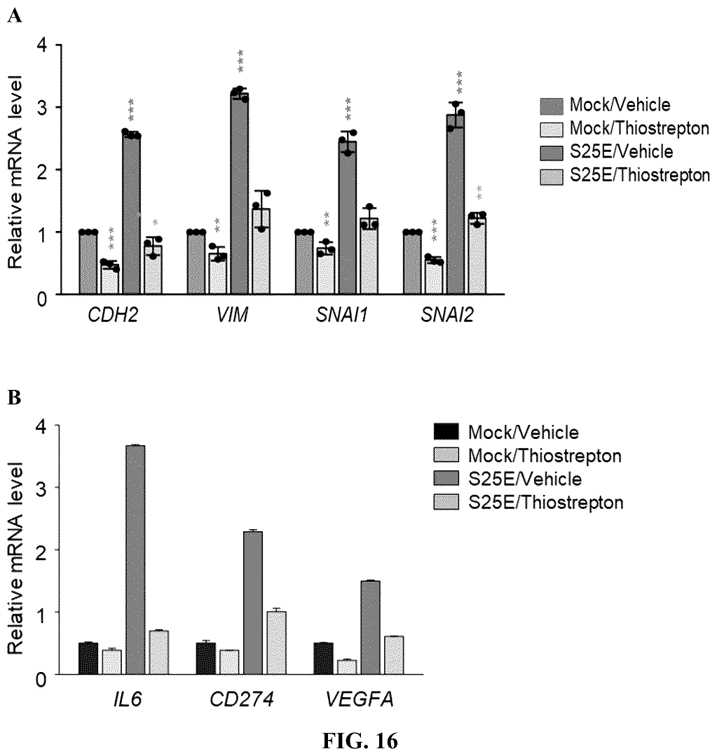

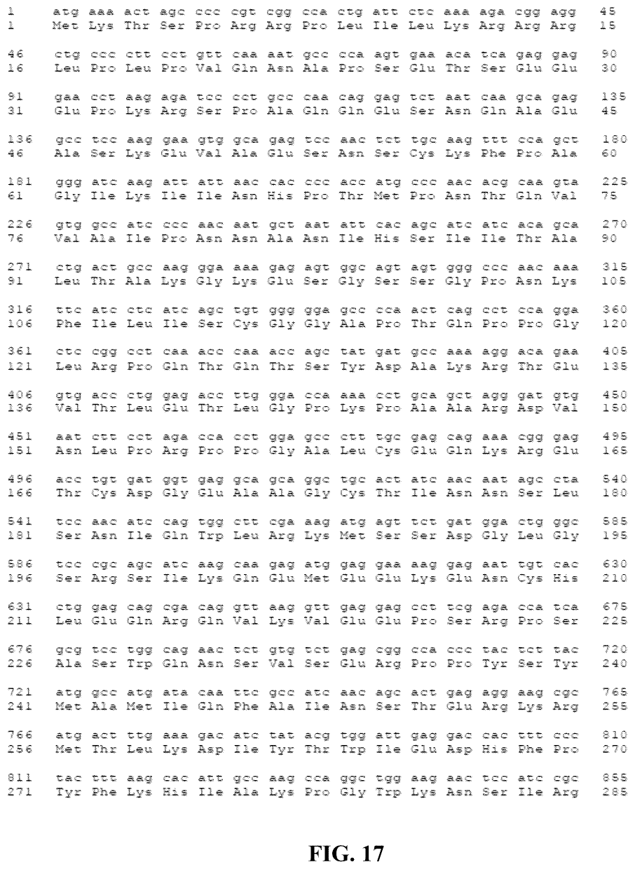

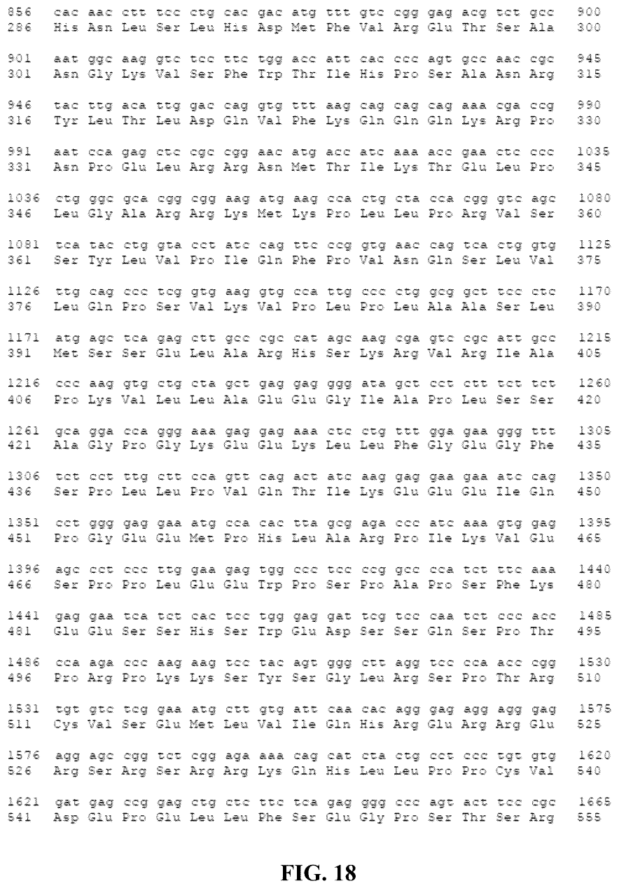

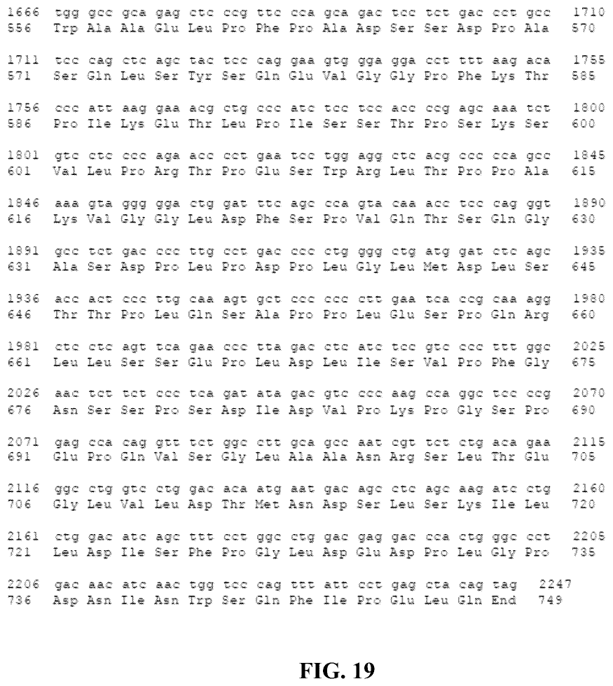

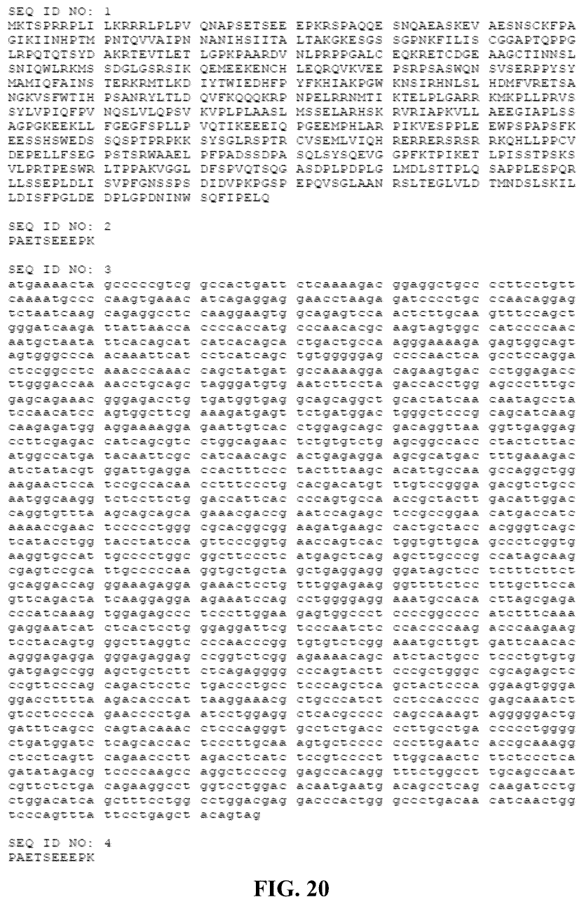

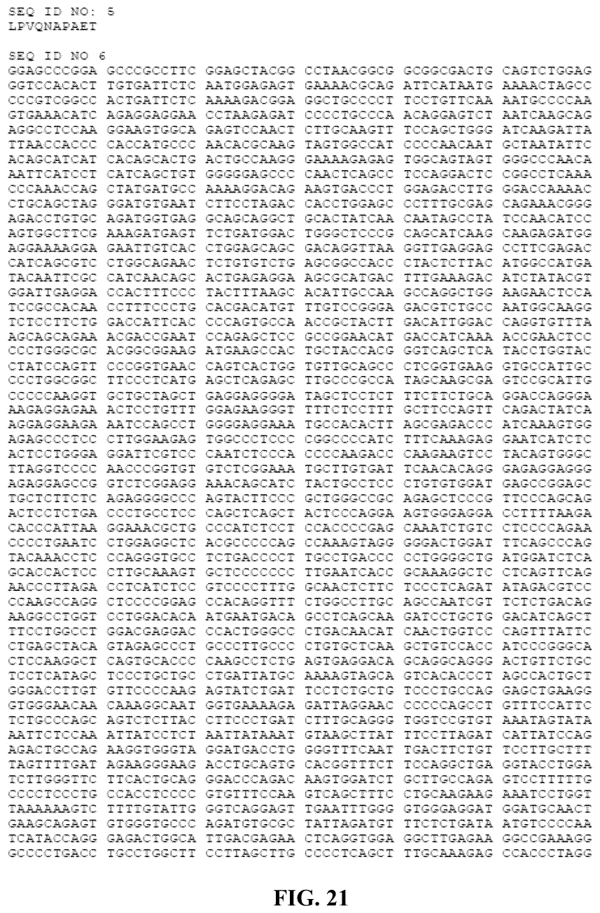

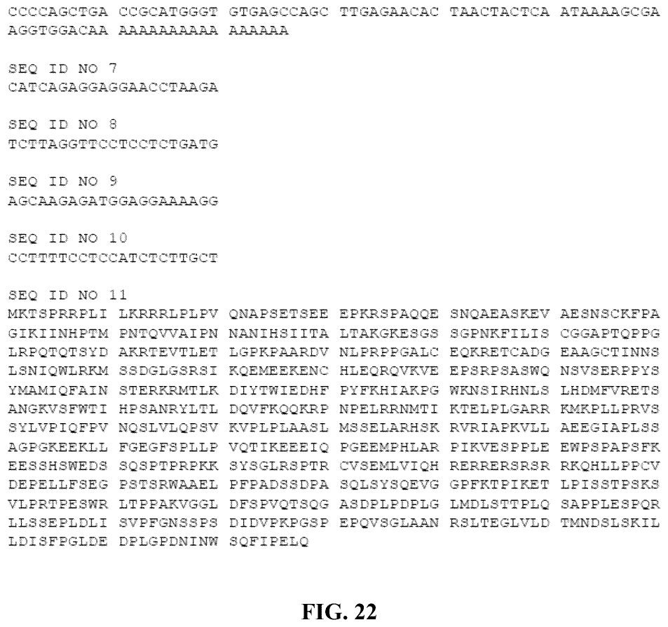

The inventor of the present invention has confirmed that the expression of FoxM1 and PLK1 is high in adenocarcinoma patients among non-small cell lung cancer patients, and is an inverse proportion to a patient survival rate, so they can be used as diagnostic markers for prognosis (FIG. 1).

In order to analyze the correlation between FoxM1 and PLK1 in non-small cell lung cancer and examine its clinical significance, the present inventor has confirmed that there was a positive correlation in the expression of the two factors in a significant range, as a result of analyzing the correlation between the mRNA expression of FoxM1 and PLK1 in lung adenocarcinoma patients with the correlation coefficient of Spearman and Pearson used big data registered in cBioPortal (FIG. 1A). In addition, as a result of analyzing the correlation with other cell cycle-related factors, the expression of FoxM1 and MKI67 was analyzed as a factor showing a high correlation coefficient with PLK1 expression (FIGS. 1B, 1C). Next, in order to confirm the clinical correlation between FOXM1 and PLK1 in non-small cell lung cancer patients, the overall survival rate of patients according to the expression level of FOXM1 and PLK1 was confirmed through big data analysis (FIGS. 1D, 1E, 1F). In non-small cell lung cancer patients, the survival rate of patients with high expression of PLK1 and FOXM1 was confirmed to be significantly lower than that of patients with low expression of PLK1 and FOXM1 (FIG. 1D).

Non-small cell lung cancer is divided into adenocarcinoma and squamous lung cell carcinoma, and according to the results of analyzing by dividing this, it could be confirmed that the survival rate of patients with high PLK1 and FOXM1 expression in lung adenocarcinoma patients is significantly lower than the survival rate of patients with low PLK1 and FOXM1 expression (FIG. 1E). In particular, as a result of re-analysis of patients with stage 3-4 where metastatic cancer was observed among lung adenocarcinoma patients, it could be confirmed that the survival rate of patients with high PLK1 and FOXM1 expression was significantly lower than the survival rate of patients with low expression of PLK1 and FOXM1 (FIG. 1F). Additionally, as a result of classifying lung adenocarcinoma patients by stage and analyzing the expression levels of FoxM1 and PLK1 using a heat map, it was confirmed that PLK1 and FOXM1 expression was higher in cancer patients compared to normal tissues and in tissues with stages 2 to 4 rather than stage 1 (FIG. 1G).

Therefore, the correlation between PLK1 and FOXM1 expression levels and patient survival rate in lung adenocarcinoma among non-small cell lung cancers could be confirmed, and in particular, it was found that high PLK1 and FOXM1 expression was a factor that lowered the patient survival rate. In addition, in patients with advanced cancer metastasis, the expression levels of PLK1 and FOXM1 were high, and thus, their value as diagnostic markers capable of determining the patient's prognosis could be recognized.

The inventor of the present invention has confirmed the correlation with cancer metastasis because the expression of FoxM1 and PLK1 is high in an environment where cancer metastasis is induced in non-small cell lung cancer cells and is proportional to the increase in PLK1 activity and phosphorylation of FoxM1 (FIG. 2).

In the analysis of the expression and survival rate of FoxM1 and PLK1 in patients with non-small cell lung cancer, and expression according to cancer stage, the present inventors have observed in clinical data that the expression of FoxM1 and PLK1 is high in metastatic cancer, so it may act as a cause to lower the survival rate, and therefore, the present inventors have tried to observe the functions of FoxM1 and PLK1 in a metastatic cancer model.

For this, in order to observe changes in the expression of PLK1 and FoxM1 and activation of PLK1 in the process of inducing epithelial-mesenchymal transition (EMT), which is a cancer metastasis cell model, by treating TGF-β, non-small cell lung cancer cell lines A549, NCI-H358, and NCI-H460 cell lines were treated with TGF-β, respectively, to induce epithelial-mesenchymal transition, and then the mRNA expression level and protein expression level were analyzed. First, in the group treated with TGF-β, an increase in the mRNA expression level of the mesenchymal markers, CDH2, SNAI1 and SNAI2 and a decrease in the mRNA expression level of the epithelial marker, CDH1 could be observed (FIGS. 2A, 2B, 2C). In addition, the protein levels of vimentin, PLK1, E-cadherin, and N-cadherin also showed the same pattern as the result of observing the result as the mRNA expression level, and in particular, it could be observed that the phosphorylated protein amount was increased higher in the TGF-β treatment group compared to the control group at the T210 residue, the active form of PLK1 (FIG. 2D). The relative changes in protein amounts indicated as a graph (FID. 2D, right figure).

In order to analyze the correlation between PLK1 and FoxM1 and observe whether phosphorylation of FoxM1 depends on EMT, A549 and NCI-H460 cells treated with TGF-β were treated with phosphatase (CIP), and the degree of phosphorylation of p-FoxM1, p-PLK1, p-TCTP was analyzed using immunoprecipitation and immunoblotting using gel retardation and a phosphorylated antibody. As a result of studying, it could be found that phosphatase treatment delayed the migration of the bands of PLK1, TCTP, and FoxM1, which were upwardly moved by TGF-β treatment. In addition, as a result of examination using a phosphorylation antibody, it was confirmed that the levels of p-FoxM1 Ser, p-PLK1 T210, and p-TCTP S46 were reduced by phosphatase treatment (FIGS. 2E, 2F). This showed that FOXM1 and PLK1 were phosphorylated during EMT induced by TGF-β treatment. Therefore, the expression of FoxM1 and PLK1 is high in an environment where non-small cell lung cancer metastasis is induced, and there is a proportional relationship with increased PLK1 activity and phosphorylation of FoxM1, indicating a correlation with phosphorylation of these factors in cancer metastasis.

The inventor of the present invention has identified sites of phosphorylation of FoxM1 by activated PLK1 and a new phosphorylation site in metastatic lung cancer cells induced by TGF-β (FIG. 3).

In order to explore the interaction between PLK1 and FoxM1 in cancer metastasis conditions induced by TGF-β treatment, Immunoprecipitation was performed. First, using the cell lysate obtained after expressing Myc-tagged FoxM1 and treating with TGF-β, PLK1 protein was precipitated with agarose beads and PLK1 antibody, and interacting proteins were analyzed through immunoblotting. As a result of studying, it was confirmed that FoxM1 and PLK1 bind to each other under a condition in which Myc-tagged FoxM1 was expressed and treated with TGF-β (FIG. 3A). In addition, as a result of observing whether endogenous FoxM1, which exists inside non-small cell lung cancer, can bind to PLK1 by TGF-β treatment in A549 cells (FIG. 3B) and NCI-H460 cells (FIG. 3C), it was observed that the binding of PLK1 and FoxM1 increased in the experimental group in which cancer metastasis was induced by TGF-β (FIGS. 3B, 3C). Accordingly, it could be observed that the interaction between PLK1 and FoxM increased during the cancer metastasis process induced by TGF-β.

Phosphorylation of FoxM1 is known to be a transcription factor that regulates expression of various factors required in a cell division phase in the cell cycle. Phosphorylation at the serine(S) site at positions 715 and 729 of FoxM1 by PLK1 has been reported, and phosphorylation at this site is known to promote cell division (FU, Zheng et al., Nature cell biology (2008) 10.9:1076-1082). No research contents have been reported on whether point mutations in these regions block the metastasis, invasion, or tumor formation of cancer cells under a condition where metastasis is increased by activated PLK1. PLK1 is a tumor phosphatase protein that is activated during the EMT process (Shin et al., Oncogene (2020) 39 (4) 767-785), and in order to determine whether FoxM1 is phosphorylated by PLK1 due to the interaction of the two proteins, a phosphatase reaction method was performed.

In the present invention, liquid chromatography mass spectrometry and phosphatase reaction method were performed to find the phosphorylation site of FoxM1 in PLK1. To prove whether PLK1, a serine/threonine phosphatase, phosphorylates FoxMl as a substrate, purified original FoxM1 and activated PLK1-T210D were added together with radiolabeled r32-P-ATP and an enzymatic reaction was performed. FoxM1 was strongly phosphorylated by active PLK1-TD at a level similar to that of TCTP, a positive control known as a substrate of PLK1 (FIG. 3D). In addition, to find the phosphorylation site of FoxM1 by PLK1, liquid chromatography mass spectrometry was performed after the phosphorylation reaction. Using this analysis, Ser-25, Ser-360, Ser-361, and Ser-393 of FoxM1 were predicted to be parts phosphorylated by PLK1 (FIG. 3E). A dephosphorylated mutant was produced by substituting the four predicted phosphorylated sites of FoxM1 and the Ser-715 residue, previously reported to phosphorylate FoxM1 by PLK1 during the cell cycle, with alanine using site-specific mutagenesis. The resulting mutant was produced by separate purification with a GST-labeled protein, which was then subjected to the same phosphatase reaction method. Among the five predicted sites of phosphorylation of FoxM1, the degree of phosphorylation of the alanine mutants at Ser-25, Ser-361, and Ser715 was significantly reduced compared to the original (FIG. 3F). As a result of this study, it could be found that PLK1 phosphorylates the Ser-25, Ser-361, and Ser-715 residues of FoxM1.

In addition, the present invention provides an effect of promoting metastasis of cancer cells by a phosphorylated point mutant protein at a new phosphorylation site of FoxM1 by PLK1 and an inhibitory effect thereof by a non-phosphorylated point mutant protein of FoxM1 (FIG. 4).

To investigate whether proteins expressing phosphorylated and non-phosphorylated point mutants of FoxM1 by PLK1 are involved in the invasion and migration of cancer cells, a stable cell line was constructed using a lentivirus system capable of inducing and controlling expression by doxycycline treatment, and then each mutant was expressed and the effects of each mutant on the invasion and migration of cancer cells were observed. Among several phosphorylated mutants, it could be observed in qRT-PCR and Western blot that the expression of N-cadherin (CDH2), an EMT marker, was increased in cells expressing S25E, a phosphorylated point mutant for Ser25. This effect was found to be reduced and inhibited in the cell group expressing S25A, a non-phosphorylated point mutant for Ser25 (FIGS. 4A, 4B). As a result of observing all mutant forms by performing a cell proliferation assay to observe the effect of the expression of each mutant on cell proliferation, it could be observed that cell proliferation was significantly increased in S715E, and it could be found that this increased more than the cell group treated with TGF-β. In addition, in the case of cells expressing the phosphorylated point mutant S25E, which had increased cell mobility, it was observed that cell proliferation did not significantly increase (FIG. 4C).

To observe metastasis of cancer cells in A549 cells expressing each protein of phosphorylated and non-phosphorylated point mutants of FoxM1, a cancer cell migration assay was performed using Transwell (FIG. 4D). As a result of the study, in the cell group expressing the phosphorylated point mutant (S25E) of the S25 residue of FoxM1, there was an approximately 6-fold increase compared to the control group, and through this, it could be found that more cancer cells were moving even compared to an approximately 4-fold increase in the TGF-treated cell group (5 ng/ml), which was a positive control. In contrast, the migration of cancer cells expressing the non-phosphorylated point mutant (S25A) protein was reduced. However, in the cell group expressing the S361 residue and S715 residue variants of FoxM1, no significant difference in migration was observed between the phosphorylated and non-phosphorylated point mutants. Therefore, it could be observed that the migration of cancer cells was increased in the cell group expressing the phosphorylated point mutant (S25E) of the FoxM1 S25 residue, while it was significantly decreased in the cells expressing S25A, a non-phosphorylated point mutant.

The inventor of the present invention has confirmed the invasion-promoting effect of cancer cells by a phosphorylated point mutant of FoxM1 and the invasion-inhibiting effect of cancer cells by a non-phosphorylated point mutant of FoxM1.

Experiments were conducted on the promoting and inhibiting effects of invasion in cancer cells using the phosphorylated and non-phosphorylated point mutants of FoxM1. The invasion of cancer cells was to be observed using an invasion assay using Matrigel (FIG. 4E). First, to evaluate the invasion ability, cells expressing each point mutant of FoxM1 were aliquoted on the Matrigel insert with serum-free medium, and serum-containing medium was aliquoted on the experimental plate and cultured for 5 days. Invaded cancer cells were observed by staining with crystal violet, dissolved in DMSO, and then absorbance was measured at a wavelength of 590 nm. As a result of the study, it was observed that the invasion of the lung cancer cell group expressing the phosphorylated point mutant protein of FoxM1 was increased compared to the control group and the original FoxM1. In particular, the highest cancer cell invasion could be observed in the S25E phosphorylation point mutant of FoxM1. On the other hand, it was observed that invasion was reduced in the lung cancer cell group expressing the non-phosphorylated point mutant protein of FoxM1. Therefore, in lung cancer cells, the invasion-promoting effect in cancer cells by the phosphorylated point mutant at the S25 residue of FoxM1 and the invasion-inhibiting effect of cancer cells by the non-phosphorylated point mutant of FoxM1 could be observed.

In addition, a wound healing assay was performed to observe the migration of cancer cells in A549 cells expressing each phosphorylated point mutant of FoxM1 (S25E, S361E, S715E) and three point simultaneous mutant (EEE) proteins (FIGS. 4F, 4G). Cell migration was observed by measuring the healing gap between cells under a microscope at 0 h, 24 h, 48 h, and 72 h, respectively (FIG. 4F). In addition, at 72 hours, the relative distance was indicated as a bar graph, with the control group set as 0 (FIG. 4G). As a result of the study, it could be found that the migration of the cell group expressing the phosphorylated point mutant (S25E) of the S25 residue of FoxM1 was increased compared to the control group, which was similar to the TGF-β treated cell group (5 ng/ml), the positive control group. It was observed that the migration of cancer cells expressing the phosphorylated point mutant (S361E) and phosphorylated point mutant (S717E) proteins showed no significant difference compared to the control group. In addition, it could be observed that the three point simultaneous mutant (EEE) had weaker migration than the point mutant (S25E), but it was observed compared to the control group. Therefore, it could be found that the migration of cancer cells was increased in the cell group expressing the phosphorylated point mutant (S25E) of the FoxM1 S25 residue, while the migration was not significantly increased in the cells expressing the other two phosphorylated point mutant S361E and S715E proteins.

Furthermore, the present invention provides the promoting effect of tumorigenesis and metastasis of cancer cells as primary cancer by phosphorylated point mutant protein at the S25 residue, a new phosphorylation site of FoxMl by PLK1, and the inhibiting effect on this by the non-phosphorylated point mutant protein of FoxM1. (FIG. 5)

To investigate whether cells expressing phosphorylated and non-phosphorylated point mutant proteins of FoxM1 by PLK1 are involved in cancer metastasis and tumorigenesis in a metastatic cancer animal model by tail vein injection using mice, BALB/c nude mice were administered with A549 cells expressing the FoxM1 S25E and S25A mutants through the tail vein injection. After breeding for 12 weeks, the animals were laparotomized to observe the degree of metastasis and tumorigenesis of cancer cells in organs (FIG. 5A). It was observed that cancer metastasis to lung organs and tumorigenesis were significantly higher in the lungs of the animal group injected with cells expressing the S25E phosphorylated point mutant of FoxM1 compared to the control, original, and animal group expressing the non-phosphorylated point mutant. On the other hand, it could be confirmed that no tumors were formed in the lungs of the animal group injected with cells expressing the non-phosphorylated point mutant of FoxM1. Therefore, the effect of promoting tumorigenesis of cancer cells by the expression of the phosphorylated point mutant protein at the new phosphorylation site by PLK1 of FoxM1 could be observed, and the effect of inhibiting the tumorigenesis of cancer cells by the non-phosphorylated point mutant protein of FoxM1 was observed.

The degree of cancer cell proliferation was to be observed through H&E (Haematoxylin and eosin) and Ki67 staining. As a result of the study, it could be observed that the degree of cancer cell proliferation was high in the FoxM1 phosphorylated point mutant experimental group and significantly low in the non-phosphorylated point mutant experimental group (FIGS. 5B and 5C). Through this, it could be confirmed that in an animal model, cancer cells expressing the phosphorylated point mutant of FoxM1 promoted cancer metastasis and tumorigenesis, while the non-phosphorylated point mutant inhibited cancer metastasis and tumorigenic ability.

Next, as a result of observing the protein level of the epithelial-mesenchymal transition marker by lysing a portion of the lung tissue, the protein level of N-cadherin, a mesenchymal marker, increased, and the level of E-cadherin decreased in the experimental group of the FoxM1 phosphorylation point mutant. In addition, as a result of confirming the expression of PD-L1, known to be involved in immune evasion and increase the tumorigenic ability of cancer cells, in each experimental group, high expression was observed in the FoxM1 phosphorylated point mutant experimental group (FIG. 5D). Additionally, similar results were confirmed not only at the protein level but also at the mRNA level (FIG. 5E). Therefore, it suggests that phosphorylation by PLK1 at S25 of FoxM1 promotes epithelial-mesenchymal transition, metastasis, and tumorigenic ability. On the other hand, it can be seen that the non-phosphorylated point mutant has an excellent effect in inhibiting epithelial-mesenchymal transition and tumorigenic ability.

To observe the degree of apoptosis of cancer cells in A549 cells expressing each phosphorylated and non-phosphorylated point mutant protein of FoxM1, the activity of caspase-3 enzyme was measured (FIG. 5F). The activity of caspase-3 enzyme was shown to be highest in cells in which the FoxM1 non-phosphorylated point mutant was overexpressed, and through this, it could be seen that overexpression of the FoxM1 non-phosphorylated point mutant induced and increased apoptosis.

Moreover, the present invention provides the effect of promoting the recruitment of monocytes to cancer cells by the phosphorylated point mutant of FoxM1 and the effect of inhibiting the polarization of monocytes into tumor-related macrophages by the non-phosphorylated point mutant of FoxM1 (FIG. 6).

Based on the research results showing that FoxM1 is involved in the migration and polarization of macrophages ((BALLI, David et al., Oncogene (2012) 31.34:3875-3888; YANG, Yang et al., Diabetes Research and Clinical Practice (2022) 184:109121), to observe whether cancer cells expressing FoxM1 S25E affect polarization of macrophage cells, THP-1 cells, which are human macrophages, and A549 cells overexpressing the FoxM1 point mutation were co-cultured for 48 hours, and then, M1 and M2 markers were observed. As a result, there was no change in the expression of the M1 markers, INOS and IL12B, but the M2 markers, IL10, CD163, CD206, TGFB1, and VEGFA were all increased in cells overexpressing the FoxMl phosphorylated point mutant (S25E) (FIG. 6A). Among them, CD206, TGFB1, and VEGFA are known to be markers of M2d-tumor-associated macrophages (M2d-TAM) (JAYASINGAM et al., Front. Oncol (2020) 9:1512), so it could be seen that upregulation of these factors in THP-1 cells co-cultured with A549 cells expressing the FoxM1 point phosphorylated mutant (S25E) differentiated the THP-1 cells into M2d-TAM.

In addition, as a result of analyzing the expression levels of M2d-inducing factors, IL4, IL6, IL10, and VEGFA in the A549 cells expressing the FoxM1 point phosphorylated variant (S25E), it was observed that the expression of these factors was increased, and it was significantly reduced in the A549 cells expressing the FoxM1 non-phosphorylated mutant (S25A) (FIG. 6B). This phenomenon was observed to be significantly increased in S25E and strongly decreased in S25A, as a result of measuring the amount of TGFB1 and VEGFA proteins in the A549 cell culture medium expressing the FoxM1 mutant by ELISA (FIG. 6C).

Next, an expression inhibition system that inhibits expression of FoxM1 was constructed to observe whether the FoxM1 point phosphorylated mutant (S25E) directly affects polarization into M2 macrophages. After co-culturing cells in which the expression of FoxM1 was inhibited and THP-1 cells, it was observed that the expression of M2 markers, CD163, CD206, and VEGFA in THP-1 cells was significantly reduced. In addition, after co-culturing THP-1 cells with cancer cells expressing the FoxM1 point phosphorylated mutant (S25E), it was confirmed that the expression of M2 markers, CD163, CD206, and VEGFA increased again in THP-1 cells compared to the control group (Mock_shCtrl) (FIG. 6D). In other words, this means that the FoxM1 phosphorylated point mutant (S25E) directly affects polarization into M2 macrophages.

In addition, as a result of analyzing CD68, a broad macrophage marker, and CD163, a tumor-related macrophage marker, in mouse lung tissue, by immunostaining, it was confirmed that the expression of the CD68, CD163 markers were highly observed in the mouse lung tissue to which the A549 cells in which the FoxM1 phosphorylated point mutant (S25E) was overexpressed were injected (FIG. 6E, 6F). On the other hand, in lung tissue injected with A549 cells overexpressing the non-phosphorylated point mutant (S25A), it was observed that the expression of CD68 and CD163 markers was significantly low. In addition, as a result of observing the expression of CD68 and CD163 proteins to observe that macrophages around the tumor were increased using lung tissue lysate by immunoblotting, it could be observed that while the expression of these tumor macrophage markers was high in mouse lung tissue injected with the A549 cells overexpressing the FoxM1 phosphorylated point mutant (S25E), the expression of these tumor macrophage markers in mouse lung tissue injected with the A549 cells overexpressing the non-phosphorylated point mutant (S25A) was lower than the control group (FIG. 6G). Through the results of this study, the effect of the FoxM1 non-phosphorylated point mutant on inhibiting polarization into tumor-associated macrophages could be observed.

In addition, the present invention provides an effect of promoting tumor immune evasion response by a phosphorylated point mutant of FoxM1 and an effect of inhibiting immune evasion response by a non-phosphorylated point mutant of FoxM1 (FIG. 7).

Tumor-related macrophages are reported to increase the survival of cancer cells by helping immune evasion of cancer cells (NOY, Roy and POLLARD, Jeffrey W., Immunity (2014) 41.1:49-61). Based on this, as a result of analyzing the survival rate of lung cancer cells by co-culturing A549 cells expressing FoxM1 phosphorylated and non-phosphorylated point mutants with mononuclear THP-1 cells, it was observed that the survival rate of A549 (A549^{S25E}) cells expressing the FoxM1 S25E phosphorylated point mutant was gradually increased, as the ratio of A549 cells: THP-1 cells was changed to 1:0, 1:0.:2, 1:4, 1:6 (FIG. 7A). On the other hand, the survival rate of A549 (A549^{S25A}) cells expressing the FoxM1 non-phosphorylated point mutant showed little change compared to the control group.

As a result of examining the mRNA expression levels of PD-1 and PD-L1, involved in immune evasion of solid cancers, in each of the above cells, it was observed that the expression of PD-1 mRNA (CD279), an immune evasion factor, was the highest in THP-1 monocytes co-cultured with A549^{S25A} cells (FIG. 7B). In addition, it was observed that the mRNA (CD274) expression of the immune evasion factor PD-L1 (CD274) was significantly increased in cells expressing the phosphorylated point mutant (A549^{S25E}). Interestingly, it was observed that the mRNA (CD274) expression of the immune evasion factor PD-L1 (CD274) in A549^{S25A} cells expressing the non-phosphorylated point mutant was reduced by about 50% compared to the control group, indicating the inhibitory effect of the non-phosphorylated point mutant of FoxM1 on expression of the immune evasion factor in cancer cells (FIG. 7C).

In addition, the effect on the tumor immune ability of T cells through co-culture with Jurkat cells, which are T cells, and cells expressing FoxM1 phosphorylated and non-phosphorylated point mutants was to be observed (FIG. 7D). The survival rate of FoxM1 phosphorylated point mutant-expressing (A549^{S25E}) cells co-cultured with Jurkat cells was observed to gradually increase as the proportion of Jurkat cells increased, but the survival rate of A549^{S25A} cells was significantly inhibited compared to the original or A549^{S25E} cells (FIG. 7D). Since it is judged that the A549^{S25E} cells may also affect the properties of T cells, the expression of CD25 and CD29 expressed on TILs in co-cultured Jurkat cells to examine the polarization into tumor-infiltrating T lymphocytes (TILs) by A549^{S25E} cells was observed. As a result, it was observed that the expression of CD25 and CD29 was significantly increased in Jurkat cells co-cultured with A549^{S25E} cells. However, the Jurkat cells co-cultured with A549^{S25A} cells showed a significant inhibitory effect compared to the original or Jurkat cells co-cultured with A549 (A549^{S25E}) cells expressing the phosphorylated point mutant (FIG. 7E). In addition, it was observed that the expression of IL6 and IL1A, which induce TIL polarization, and CD274, a tumor immune evasion factor, was increased in A549^{S25E} cells, but the expression of these factors was observed to be significantly low in A549^{S25A} cells (FIG. 7F). Additionally, it was observed that the A549^{S25A} cells had an inhibitory effect on the tumor immune evasion response caused by TAMs or TILs in the tumor microenvironment through analysis of changes in survival rate after co-culturing the THP-1 cells or Jurkat cells with the A549 cells overexpressing FoxM1 phosphorylated and non-phosphorylated point mutants (FIG. 7G). Therefore, the present invention provides the effect of inhibiting tumor immune evasion response by the non-phosphorylated point mutant of FoxM1.

In addition, the present invention provides an inhibitory effect on cancer cell invasion and migration and polarization into tumor-associated macrophages (TAM) by the non-phosphorylated point mutant peptide of FoxM1 (FIG. 8).

It is known that macromolecules such as proteins and nucleic acids are difficult to be delivered into cells because they cannot penetrate the cell membrane. Various technologies have been developed to deliver proteins into cells, and the most representative technology includes cell-penetrating peptide (CPP). CPP is a short-length peptide consisting of between 8 and 30 amino acids and are classified as those with cationic, amphipathic, or hydrophobic properties. It is known to be able to pass through cell membranes. Tat (trans-activator of transcription), a protein contained in human immunodeficiency virus type-1 (HIV-1), is observed to have a cell-penetrating function, and this function is achieved by properties of a protein transduction domain (PTD), a middle region of the Tat protein consisting of 11 amino acids, and a clear mechanism has not been known yet. Since PTD was identified, various CPPs derived from proteins existing in nature or artificially designed and synthesized have been reported (Frankel, A. D. et al., Cell. (1988) 55:1189-1193). HIV-1 Tat has a short cationic domain, and through subsequent studies, the peptide sequence of this domain (pTAT, YGRKKRRQRRR) was identified (Heitz, F. et al., Br. J. Pharmacol. (2009) 157:195-206; Vives, E., P. et al., J. Biol (1997) 272:16010-16017. Using the TAT, the inhibitory effect on metastatic cancer by delivering a peptide comprising a FoxM1 point mutation into cells was to be evaluated.

First, three peptides were synthesized by attaching TAT (YGRKKRRQRRR), a type of CPP (Cell-Penetrating Peptide), to infiltrate the FoxM1 phosphorylated point mutant peptide (QNAPAETSEE) into cells (FIG. 8A). In order to evaluate the efficacy of the three peptides produced, the inhibitory effect on apoptosis using the enzymatic activity of caspase-3 and cell migration using a migration assay was observed (FIGS. 8B, 8C). As a result of measuring the enzyme activity of caspase-3 after treating A549 cells with each FoxM1 non-phosphorylated point mutant peptide (5 μM FoxM1-S25A peptide) for 48 hours, it could be observed that treatment with each of the three peptides increased apoptosis, and in particular, it could be confirmed that the apoptotic effect was significant in peptide #1 and peptide #3. Furthermore, under a condition of overexpression of the phosphorylated point mutant of FoxM1 in A549 cells, the inhibitory effect on migration of cancer cells was observed by treatment with each of the three non-phosphorylated FoxM1 point mutant peptides (5 μM FoxM1-S25A peptide) (FIG. 8C). All the three peptides were observed to have an inhibitory effect on the migration of cancer cells, and in particular, it was observed that the inhibitory effect was the greatest in peptide #1.

In order to investigate whether peptide #1 determined to be the most effective among the three peptides penetrates into cells, it was produced by attaching an FITC fluorescent material at the C-terminal side of the peptide (FIG. 10A), and it was observed that the peptide penetrated into cells after 24 hours of peptide treatment (FIG. 10B). It was observed that treatment of the peptide penetrated into cells under the cancer metastasis conditions induced by TGF-β inhibited changes in mRNA expression of an epithelial mesenchymal transition marker (EMT marker) (FIG. 10C). Additionally, it could be confirmed the effect of treatment of the FoxM1 non-phosphorylated point mutant peptide in inhibiting migration of cancer cells increased under the metastasis environment treated with TGF-β through a cell migration assay (FIG. 10D).

In addition, it was observed that the mRNA expression of CDH2 and vimentin, which are mesenchymal transition markers increased by overexpression of FoxM1, by treatment of the FoxM1 non-phosphorylated point mutant peptide to lung cancer cell A549 overexpressing the FoxM1 phosphorylated point mutant, and conversely, the decrease in mRNA expression of CDH1, an epithelial marker was increased by peptide treatment (FIG. 10E). Furthermore, the inhibitory effect of the migration of cancer cells by treatment of the FoxM1 non-phosphorylated point mutant peptide (5 μM FoxM1-S25A peptide) under the condition of overexpression of the FoxM1 phosphorylated point mutant in A549 cells was observed (FIG. 10F).

Additionally, in order to observe a role of the FoxM1 non-phosphorylated point mutant peptide on tumor-associated macrophage polarization and tumor cell immune evasion ability, changes in STAT1, VEGFA, c-fos, IL6, CD274 mRNA were observed after treatment of the FoxM1 non-phosphorylated point mutant peptide (5 μM FoxM1-S25A peptide) under the condition of overexpression of the FoxM1 phosphorylated point mutant in A549 cells. It was observed that the STAT1, VEGFA, c-fos, IL6, CD274 mRNA expression increased by overexpression of FoxM1 was inhibited by peptide treatment (FIG. 10G). Accordingly, it could be confirmed that the FoxM1 non-phosphorylated point mutant peptide had the inhibitory effect on the tumor-associated macrophage polarization and tumor cell immune evasion ability.

In addition, the present invention provides an anticancer therapeutic agent against not only primary cancer but also metastatic cancer comprising a FoxM1 inhibitor or a Fox1 protein and peptide comprising a non-phosphorylated point mutant as an active ingredient.

The anticancer agent comprising a protein and a peptide which comprise a FoxM1 point mutant as an active ingredient of the present invention can be used for prevention and treatment of various kinds of cancers with excessive expression of FoxM1, in particular, various kind of solid cancers such as lung cancer (LIANG, Sheng-Kai, et al. Oncogene, 2021, 40.30:4847-4858), breast cancer (ZIEGLER, Yvonne, et al. NPJ breast cancer, 2019, 5.1:1-11), liver cancer (YU, Chun-Peng, et al. Molecular medicine reports, 2017, 16.4:5181-5188), prostate cancer (Kalin, Tanya V., et al. Cancer research 66.3 (2006): 1712-1720), colorectal cancer (Yoshida, Yuichi, et al. Gastroenterology 132.4 (2007): 1420-1431), brain cancer (Liu, Mingguang, et al. Cancer research 66.7 (2006): 3593-3602), and leukemia, and the like (XU, Xin-Sen, et al. Asian Pacific Journal of Cancer Prevention, 2015, 16.1:23-29.), and in addition, it can be also used for prevention and treatment of metastatic solid cancers caused by being metastasized from primary cancer (WANG, Yi-Wei, et al. Journal of biomedical science, 2022, 29.1:1-23).

The protein or polypeptide described herein refers to a protein or polypeptide comprising an amino acid sequence represented by SEQ ID NO: 1 or SEQ ID NO: 2, or comprising an amino acid sequence having at least 80% homology, at least 85% homology, at least 86% homology, at least 87% homology, at least 88% homology, at least 89% homology, at least 90% homology, at least 91% homology, at least 92% homology, at least 93% homology, at least 94% homology, at least 95% homology, at least 96% homology, at least 97% homology, at least 98% homology, or at least 99% homology, which has the substantially same biological properties as SEQ ID NO: 1 or SEQ ID NO: 2.

As known in the art, the term “percent identity” is the relationship between two or more polypeptide sequences or two or more polynucleotide sequences as determined by comparing sequences. In addition, in the art, “identity” means the degree of sequence correspondence between polypeptide or polynucleotide sequences, as determined by correspondence between character strings of the sequences, in some cases. “Identity” and “similarity” can be easily calculated by known methods, including those described in Computational Molecular Biology ((Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991), but not limited thereto. A preferred method for determining identity is designed to provide optimal matching between tested sequences. The method of determining identity and similarity is codified in a publicly available computer program. Sequence alignment and percent identity calculation can be performed using a sequence analysis software such as Megalign program of LASERGENE bioinformatics computing suite (DNASTAR Inc., Madison, WI). Multiple alignment of sequences can be performed using Clustal alignment method (Higgins et al., CABIOS. 5:151 (1989)) with a default parameter (GAP PENALTY=10, GAP LENGTH PENALTY=10). The default parameter for pairwise alignment using Clustal method may be selected from KTUPLE 1, GAP PENALTY-3, WINDOW=5 and DIAGONALS SAVED=5. As known in the art, “similarity” between two kinds of polypeptides is measured by comparing an amino acid sequence, and a conserved amino acid substitution of a polypeptide with the second polypeptide. Herein, the identity or homology for these sequences is defined as a percentage of amino acid residues in a candidate sequence that is identical to a known peptide, by aligning sequences and if necessary, introducing a gap to achieve the maximum homology (%) and not considering any conservative substitution as a part of sequence identity. N-terminal, C-terminal or internal extension, deletion or insertion in the peptide sequence will not be interpreted as affecting homology.

The term “homology” refers to the percentage of identity between portions of two polynucleotides or two polypeptides. Correspondence between sequences from one part to another can be determined by techniques known in the art. For example, homology can be determined by aligning sequence information and directly comparing sequence information between two polypeptide molecules using a readily available computer program. Otherwise, homology can be determined by hybridizing polynucleotides under conditions that form a stable duplex between homologous regions, followed by cleavage using a single-stranded specific nuclease and size determination of the cleaved fragments.

Accordingly, the term “sequence similarity” in all its grammatical forms refers to the degree of identity or correspondence between nucleic acid or amino acid sequences that may or may not have a common evolutionary origin (See Reeck et al., Cell 50:667 (1987)). In one specific embodiment, two DNA sequences are “substantially homologous” or “substantially similar”, when about 50% (e.g., at least about 75%, 90%, or 95%) of the nucleotides match at least over a defined length of DNA sequence. Substantially homologous sequences can be identified by comparing sequences using a sequence data bank or, for example, a standard software available in Southern hybridization experiments under stringent conditions as defined for a particular system. Defining appropriate hybridization conditions is within the technical category of the art (e.g., See Sambrook et al., 1989).

As used in the present specification, “substantially similar” refers to a nucleic acid fragment in which a change in at least one nucleotide base causes substitutions of at least one amino acid, but it does not affect functional properties of the protein encoded by the DNA sequence. “Substantially similar” refers to a nucleic acid fragment in which a change in at least one nucleotide base does not affect the ability of the nucleic acid fragment mediating a change in gene expression by antisense or co-suppression method. “Substantially similar” also refers to a modification of the nucleic acid fragment of the present invention, such as deletion or insertion of at least one nucleotide base that does not substantially affect functional properties of transcripts obtained. Therefore, the present invention is interpreted to comprise more than the illustrated specific sequences. Each modification suggested belongs to common technologies in the art, as determining retention of biological activity of encoded products.

Moreover, those skilled in the art recognizes that the substantially similar sequences included by the present invention are defined by ability to be hybridized with the sequences illustrated in the present specification under stringent conditions (washing with 0.1X SSC, 0.1% SDS, 65° C. and 2X SSC, 0.1% SDS, and then 0.1X SSC, 0.1% SDS). The substantially similar nucleic acid fragments of the present invention are nucleic acid fragments at least about 70%, 80%, 90% or 95% identical to the DNA sequence of the nucleic acid fragment reported in the present specification.

“Variant” of a polypeptide or protein refers to any analogue, fragment, derivative or mutant which is derived from a polypeptide or protein and maintains at least one biological property of the polypeptide or protein. Different variants of the polypeptide or protein may be present in nature. These variants may be variations of allelic genes characterized by having a different nucleotide sequence of a structural gene encoding the protein, or may include differential splicing or post-translational modification. Those skilled in the art may produce a variant having one or multiple amino acid substitutions, deletions, additions, or replacements. These variants may comprise (a) variants in which one or more amino acid residues are substituted with conservative or non-conservative amino acids, (b) variants in which one or more amino acids are added to a polypeptide or protein, (c) variants in which at least one of amino acids comprises a substituent group, and (d) variants in which a polypeptide or protein is fused with another polypeptide such as serum albumin: among them.

In addition, conservative variants mean amino acid sequences with sequence changes that do not affect biological functions of the protein adversely. When the changed sequences interfere with or destroy biological functions related to the protein, it is described that a substitution, insertion or deletion adversely affects the protein. For example, the total charges, structure or hydrophobicity-hydrophilicity of the protein may not affect biological activity adversely and be changed. Therefore, the amino acid sequences may not adversely affect, for example, biological activity of the protein, and be changed to make the peptide show higher hydrophobicity or hydrophilicity. Techniques for obtaining such variants, including genetic (inhibition, deletion, mutation, etc.), chemical and enzymatic techniques are known to those skilled in the art.

“Conservative amino acid substitution” is one in which an amino acid residue is replaced by an amino acid residue. Families of amino acid residues having a similar side chain. These families include amino acids with a basic side chain (e.g., Lys, Arg, His), amino acids with an acidic side chain (e.g., Asp, Glu), amino acids with a uncharged polar side chain (e.g., Gly, Asn, Gln, Ser, Thr, Tyr, Cys), amino acids with a non-polar side chain (e.g., Ala, Val, Leu, Ile, Pro, Phe, Met, Trp), amino acids with beta-branched side chain (e.g., Thr, Val, Ile) and amino acids with an aromatic side chain (e.g., Tyr, Phe, His).

Proteins or polypeptides may undergo a post-translational modification process such as phosphorylation. Protein phosphorylation is a reversible process, and is catalyzed by protein kinase. In mammals, most phosphorylation occurs on a Ser, Thr, or Tyr residue in proteins or amino acids. In one example of the present invention, to inhibit protein phosphorylation, a phosphorylation residue, Ser may be substituted with a non-phosphorylation amino acid, Gly, Ala, Val, Ile, Leu, Met, Phe, Trp, Asn, Gln, Cys, Pro, Arg, His, or Lys, and in one example of the present invention, it may be substituted with Gly, Ala, Val, or Cys. In one example of the present invention, Ser is substituted with Ala.

DNA “coding sequence” refers to a double-stranded DNA sequence that can be transcribed and translated to a polypeptide in vitro or in vivo when a polypeptide is encoded and is placed under the control of an appropriate regulatory sequence. “Appropriate regulatory sequence” refers to a nucleotide sequence located upstream (5′ non-coding sequence), inside, or downstream (3′ non-coding sequence), and this affects transcription, RNA processing or stability or translation of the associated coding sequence. The regulatory sequence may comprise a promoter, a translation leader sequence, an intron, a polyadenylation recognition sequence, an

RNA processing site, an effector binding site, and a stem-loop structure. The boundaries of the coding sequence are determined by a start codon at the 5′ (amino) end and a translation stop codon at the 3′ (carboxyl) end. The coding sequence may comprise cDNA from mRNA, a genomic DNA sequence, and even a synthetic DNA sequence, but not limited thereto. If the coding sequence is to be expressed in eukaryotic cells, polyadenylation signals and transcription termination sequences may be generally positioned at the 3′ side of the coding sequence.

In the present invention, the term, “recombinant vector” is an expression vector capable of expressing a target protein in a suitable host cell, and refers to a genetic construct comprising essential regulatory elements operably linked to express a gene insert.

The term “plasmid” refers to an extrachromosomal element that often carries genes which are not a part of main mechanisms of cells, and are present commonly in a circular double-stranded DNA molecule. Such elements may be autonomously replicating sequences, genetic integration sequences, phage or nucleotide sequences, linear, circular or supercoiled single-or double-stranded DNA or RNA, from any source, where a number of nucleotide sequences are linked or recombined to form a unique construct that can introduce a promoter fragment and a selected gene product into a cell with an appropriate 3′ untranslated sequence. In general, the plasmid comprises an origin of replication that functions in bacterial host cells (e.g., E. coli (Escherichia coli)), and a selectable marker for detection of bacterial host cells including the plasmid.

The term “expression vector” refers to a vector, plasmid, or carrier designed to transform a host after expressing an inserted nucleic acid sequence. The cloned gene, that is, the inserted nucleic acid sequence is generally placed under the control of regulatory elements such as a promoter, a minimal promoter, an enhancer, and the like. There are numerous Initiation regulatory regions or promoters useful for inducing expression of nucleic acids in a desired host cell, and are well known to those skilled in the art. Substantially, any promoter that can induce expression of these genes includes virus promoters, bacteria promoters, animal promoters, mammalian promoters, synthetic promoters, constitutive promoters, tissue specific promoters, pathogenesis or disease-related promoters, developmental specific promoters, inducible promoters, and light regulated promoters, but not limited thereto; and includes SV40 early (SV40) promoter regions, promoters contained in the 3′ long terminal repeat (LTR) of Rous sarcoma virus (RSV), E1A or major late promoters (MLP) of adenovirus (Ad), human cytomegalovirus (HCMV) immediate early promoters, herpes simplex virus (HSV) thymidine kinase (TK) promoters, baculovirus IE1 promoters, elongation factor 1 alpha (EF1) promoters, glyceraldehyde-3-phosphate dehydrogenase (GSPDH) promoters, phosphoglycerate kinase (PGK) promoters, ubiquitin C (Ubc) promoters, albumin promoters, mouse metallothionein-L promoters and regulatory sequences of transcription regulatory regions, ubiquitous promoters (HPRT, vimentin, β-actin, tubulin, etc.), intermediate filaments (desmin, neurofilament, keratin, GFAP, etc.), promoters of therapeutic genes (such as MDR, CFTR or factor VIII forms), pathogenesis-or disease-related promoters, and promoters that show tissue specificity, such as elastase I gene regulatory region, which is active in pancreatic acinar cells, and have been used in transformed animals; insulin gene regulatory regions which are active in pancreatic beta cells, immunoglobulin gene regulatory regions that are active in lymphoid cells, mouse breast cancer virus regulatory regions that are active in testicular, breast, lymphatic system and macrophages; albumin gene, Apo AI and Apo AII regulatory regions active in the liver, alpha-fetoprotein gene regulatory regions active in the liver, alpha1-antitrypsin gene regulatory regions active in the liver, beta-globin gene regulatory regions active in bone marrow cells, myelin basic protein regulatory regions active in oligodendrocyte cells in the brain, myosin light chain-2 gene regulatory regions active in skeletal muscle and gonadotropic releasing hormone active in the hypothalamus, pyruvate kinase promoters, villin promoters, fatty acid binding intestinal protein promoters, promoters of smooth muscle cell B-actin and the like, but not limited thereto.

“Nucleic acid”, “nucleic acid molecule”, “oligonucleotide” and “polynucleotide” are interchangeably used, and refer to polymeric forms of phosphate ester of ribonucleosides (adenosine, guanosine, uridine or cytidine; “RNA molecule”) or deoxyribonucleosides (deoxyadenosine, deoxyguanosine, deoxythymine, or deoxycytidine; “DNA molecule”) or any phosphate ester analogues thereof such as phosphorothioate and thioester, which is a single-stranded form or double-stranded helix. Double helix DNA-DNA, DNA-RNA and RNA-RNA helices are possible. The term, nucleic acid molecule, in particular, DNA or RNA molecule refers only to primary and secondary structures of the molecule, and is not limited to any specific tertiary form. Therefore, this term includes double-stranded DNA found in linear or circular DNA molecules (e.g., restriction enzyme fragments), plasmids, supercoiled DNA and chromosomes, among them. When discussing the structure of a specific double-stranded DNA molecule, the sequence may be described in the present specification according to general regulations which present the sequence only in the 5′ to 3′ direction along a non-transcribed DNA strand (i.e., strand with the sequence matching mRNA). “Recombinant DNA molecule” is a DNA molecule that has undergone molecular biological manipulation. DNA includes cDNA, genomic DNA, plasmid DNA, synthetic DNA and semi-synthetic DNA, but not limited thereto.

The term “transfection” refers to uptake of exogenous or heterologous RNA or DNA by a cell. When exogenous or heterologous RNA or DNA is introduced into a cell, the cell is “transfected” by this RNA or DNA. When the transfected RNA or DNA brings a phenotypic change, the cell is “transfected” by the exogenous or heterologous RNA or DNA. The RNA or DNA transforming can constitute the genome of the cell by being inserted (covalently linked) into the stained DNA.

The term “expression” refers to biological production of products encoded by a coding sequence. In most cases, a DNA sequence comprising a coding sequence is transcribed to form messenger-RNA (mRNA). Subsequently, the messenger RNA is translated to form polypeptide products having relevant biological activity. In addition, the expression process may include an additional processing step for RNA transcription products (e.g., splicing to remove introns), and/or post-translational processing of polypeptide products.

The present invention provides a host cell comprising the vector of the present invention. The host cell includes prokaryotic (e.g., bacteria) and eukaryotic (e.g., fungi, yeast, animals, insects, plants) cells, and may be any cell suitable for expression of fusion proteins. Suitable prokaryotic host cells include E. coli (e.g., DH5, HB101, JM109 or W3110 line), Bacillus, Streptomyces, Salmonella, Serratia and Pseudomonas species, but not limited thereto. Suitable eukaryotic host cells include COS, CHO, HepG-2, CV-1, LLCMK2, 3T3, HeLa, RPMI8226, 293, BHK-21, Sf9, Saccharomyces, Pichia, Hansenula, Kluyveromyces, Aspergillus or Trichoderma species, but not limited thereto.

The peptide, protein, polypeptide, nucleic acid, siRNA, shRNA, or miRNA and an additional material, for example, an immune checkpoint inhibitor described herein may be administered as a pharmaceutical composition or drug for therapeutic or preventive treatment, and may be administered as any appropriate pharmaceutical composition form which can comprise a pharmaceutically acceptable carrier, and selectively, can comprise at least one adjuvant, stabilizer, and the like. In one embodiment, the pharmaceutical composition is to be used for therapeutic or preventive treatment, for example, treating or preventing diseases such as cancer disease as described herein.

The term “pharmaceutical composition” relates to a formulation comprising a therapeutically effective substance, preferably, with a pharmaceutically acceptable carrier, diluent and/or excipient. The pharmaceutical composition is useful for lowering, preventing or treating the severity of disease or disorder by administering the pharmaceutical composition into a subject. The pharmaceutical composition is also known in the art as a pharmaceutical formulation. In the context of the present invention, the pharmaceutical composition includes peptides, proteins, polypeptides, RNAs, or RNA particles as described herein.

The pharmaceutical composition of the present specification may comprise at least one adjuvant, or be administered with at least one adjuvant. The term “adjuvant” refers to a compound which extends, enhances or accelerates an immune response. The adjuvant includes a heterogeneous group such as oil emulsions (e.g., Freund's adjuvant), mineral compounds (e.g., alum), bacterial products (e.g., pertussis toxin) or immune-stimulating complexes. Examples of the adjuvant non-restrictively include cytokines such as LPS, GP96, CpG oligodeoxynucleotides, growth factors and monokines, lymphokines, interleukins, and chemokines. The cytokines may be IL1, IL2, IL3, IL4, IL5, IL6, IL7, IL8, IL9, IL10, IL12, IFNα, IFNγ, GM-CSF, LT-a. Additional known adjuvants are aluminum hydroxide, Freund's adjuvant or oils such as Montanide® ISA51. Other adjuvants suitable for use in the present specification include lipopeptides such as Pam3Cys.

The pharmaceutical composition according to the present specification is generally applied as a “pharmaceutically acceptable formulation”, in a “pharmaceutically effective dose”.

The term “pharmaceutically acceptable” means non-toxicity of a substance that does not interact with the action of active ingredients of a pharmaceutical composition.

The term “pharmaceutically effective dose” or “therapeutically effective dose” means the amount that achieves the desired response or desired effect alone or in combination with additional administration. In case of treating a specific disease, the desired response preferably means inhibition of progression of the disease. This includes slowing down the rate of progression of the disease, and in particular, it includes stopping or reversing the progression of the disease. The desired response in the treatment of the disease may also be delaying initiation or preventing initiation of the disease or the condition. The effective dose of the composition described herein may be determined depending on the condition being treated, severity of the disease, individual characteristics of the patient including age, body condition, height and body weight, treatment period, (if present) type of accompanied therapy, specific administration route and similar factors. Therefore, the dosage of the composition described herein can be determined according to these various properties. When the response in a patient is not sufficient with the first administration, a dose higher than this (or effectively, higher dose achieved by other, more local administration route).

The pharmaceutical composition of the present specification can comprise a salt, a buffer, a preservative, and selectively, other therapeutic substances. In one embodiment, the pharmaceutical composition of the present specification comprises at least one pharmaceutically acceptable carrier, diluent and/or excipient.

The preservative suitable for using in the pharmaceutical composition of the present specification, non-restrictively, includes benzalkonium chloride, chlorobutanol, paraben, and thimerosal.

As used herein, the term “excipient” can be present in the pharmaceutical composition of the present specification, but refers to substances other than active ingredients. Examples of the excipient, non-restrictively, include carriers, binders, diluents, lubricants, thickeners, surfactants, preservatives, stabilizers, emulsifiers, buffers, fragrance ingredients or coloring agents.

The term “diluent” means a substance diluting and/or thinning. In addition, the term “diluent” includes any one or more of fluid, liquid, or solid suspensions and/or mixed media. Examples of the appropriate diluent include ethanol, glycerol, and water.

The term “carrier” refers to a component that may be natural, synthetic, organic or inorganic, combined with active ingredients to make administration of a pharmaceutical composition easy, enhance it or perform administration. As used herein, the carrier may be at least one compatible solid or liquid filler, diluent or encapsulating material, which is suitable for administration to a subject. The suitable carrier, non-restrictively, includes sterile water, Ringer's solution, Ringer lactate, sterile sodium chloride solution, isotonic saline solution, polyalkylene glycol, hydrogenated naphthalene, and especially, biocompatible lactide polymers, lactide/glycolide copolymers or polyoxyethylene/polyoxypropylene copolymers. In one embodiment, the pharmaceutical composition of the present specification includes isotonic saline solution.

The pharmaceutically acceptable carrier, excipient, or diluent for therapeutic use are well known in the pharmaceutical art, and for example, they are described in Remington's Pharmaceutical Sciences, Mack Publishing Co. (A. R Gennaro edit. 1985).

The pharmaceutical carrier, excipient or diluent can be selected depending on the intended administration route and standard pharmaceutical practice.

In one embodiment, the pharmaceutical composition described herein may be administereed intravenously, intraarterially, subcutaneously, intradermally, or intramuscularly. In a specific embodiment, the pharmaceutical composition is formulated for local administration or systemic administration. Systemic administration can include enteral administration or parenteral administration, accompanying absorption through the gastrointestinal tract. As used herein, “parenteral administration” means administering by any way other than being conducted through the gastrointestinal tract, as intravenous injection. In a preferred embodiment, the pharmaceutical composition is formulated for systemic administration. In another preferred embodiment, systemic administration is by intravenous administration. In one embodiment in all aspects of the present invention, the FoxM1 variant, fragment tehreof, or nucleic acid encoding the same is systemically administered.

As used herein, the term “co-administration” means administering various kinds of compounds or compositions into the same patient. The various kinds of compounds or compositions may be simultaneously, essentially simultaneously, or sequentially.

The material, composition and method described herein, can be used for treating a subject with a disease characterized by presence of a cell with a disease, for example, a disease expresing an antigen. In particular, a preferred disease is a cancer disease. For example, if an antigen is derived from a virus, the material, composition and method can be useful for treatment of viral diseases caused by the virus. If an antigen is a tumor antigen, the material, composition and method are useful for treatment of a cancer disease, and herein, cancer cells express the tumor antigen.

The material, composition and method described herein can be used for therapeutic or preventive treatment of various diseases, and herein, as described herein, the support of immune effector cells and/or activity of immune effector cells are advantageous for patients with cancers and infectious diseases. In one embodiment, the material, composition and method described herein are useful for preventive and/or therapeutic treatment of antigen-associated diseases.

The term “disease” refers to abnormal conditions affecting a subject's body. The disease is often interpreted as a medical condition associated with specific symptoms. The disease may be caused by factors originated from external causes such as infectious diseases, or may be caused by internal dysfunction such as autoimmune diseases. In humans, “disease” is used to refer any condition in which the disease causes pain, dysfunction, suffering, social problems, or death, or similar problems, in a subject suffering from a disease in contact with a subject in a broader meaning. In a broader meaning, this sometimes includes injuries, disabilities, disorders, syndromes, infections, single symptom, deviant acts, and structural and functional atypical modifications, and in another context and for another purpose, this may be considered a distinguishable category. The disease generally affects a subject not only physically but also emotionally, since a subject's perspective on life and personality can be changed, in case of living with various diseases.

In the present context, the term “treatment”, “treating” or “therapeutic intervention” means managing and caring of a subject for the purpose of eradicating conditions such as a disease or disorder. This term is intended to include the full range treatment for a certain condition from which a subject is suffering, as administration of a therapeutically effective compound, in order to not only alleviating symptoms or complications, and/or delaying progression of a disease, disorder or condition, and/or alleviating or reducing symptoms and complications, curing or eliminating a disease, disorder or condition, but also preventing a condition, and herein, prevention will be understood by managing and caring a subject for the purpose of eradicating a disease, condition or disorder, and includes administration of an active compound to prevent initiation of symptoms or complications.

The term “therapeutic treatment” means any treatment of improving health conditions of a subject and/or extending (increasing) the lifespan of a subject. The treatment may be elimination of a disease in a subject, stop or slowing of progression of a disease in a subject, inhibition or slowing of progression of a disease in a subject, reduction of frequency or severity of symptoms in a subject, and/or reduction of recurrence in a subject currently suffering from or previously suffering from a disease.

The term “prophylactic treatment” or “preventive treatment” means any treatment intended to prevent a disease from occurring in a subject. The term “prophylactic treatment” or “preventive treatment” is interchangeably used herein.

The terms “indicidual” and “subject” are interchangeably used herein. These terms, refer to humans or other mammals (e.g., mice, rats, rabbits, dogs, cats, cows, pigs, sheep, horses, or primates) who may be taken with or vulnerable to a disease or disorder (e.g., cancer), but they may be not takedn wiwth the disease or disorder. In many embodiments, the obejct is a human. Unless otherwise specified, the terms “individual” and “subject” do not mean a specific age, and therefore, encompass adults, the elderly, children, and newborns. In an embodiment of the present specification, “subject” or “individual” is a “patient”.

The term “patient” means an individual or entity in need of treatment, specifically, an individual or subject affected by a disease.

In one embodiment of the present specification, the purposes are to cause an immune response against cells affected by a disease expressing an antigne such as cancer cells expressing a tumor antigen, to reduce migration, invasion, and proliferation for tumor cells, and to treat a disease such as cancer diseases involving cells expressing an antigen such as a tumor anigen.

As used herein, “immune response” refers to an integrated body response to an antigen or a cell expressing the antigen, and refers to a cellular immune response and/or humoral immune response.

“Cell-mediated immunity”, “cellular immunity”, “cellular immune response” or similar terms mean including a cellular response to a cell characterized by expression of an antigen, particularly, characterized by suggesting an antigen with class I or class II MHC. The cellular response relates to a cell called a T cell or T lymphocyte that acts as either “helpers” or “killers”. Helper T cells (also referred to as CD4+ T cells) play a central role by controlling an immune response, and killer cells (also referred to as cytotoxic T cells, cytolytic T cells, CD8⁺ T cells or CTL) kill cells affected by a disease such as cancer cells to prevent generation of cells afftected by more diseases.

The prsent specification considers an immune response that may be protective, prophylactic, preventive, and/or therapeutic. As used herein, “induce (or inducing) an immune response” may mean that there is no immune response against a specific antigen before inducing, or that there is an immune response against a specific antigen is present at a basal level before inducing and is intensified after inducing. Therefore, “induce (or inducing) an immune response” incldues “intensify (or intensifying) an immune response”.

The term “immunotherapy” refers to treatment of a disease or condition by inducing or enhancing an immune response. The term “immunotherapy” includes antigen immunization and antigen vaccination.

The term “immunization” or “vaccination” refers to a process of administering an antigen into an individual for a purpose to induce an immune response, for example, for therapeutic or prophylactic reasons.

The term “macrophage” refers to a subgroup of phagocytes created by polarization of monocytes. Macrophages activated by inflammation, immune cytokines, or microbial products non-specifically perform phagocytosis, and kill exogenous pathogens in the macrophages by hydrolytic and oxidative attacks to degrade the pathogens. Peptides derived from degraded proteins are presented on the cell surface of macrophages, and this can be recognized by T cells, and they can directly interact with an antibody on the B cell surface to activate T and B cells and additionally stimulate an immune response. Macrophages belong to the class of antigen-presenting cells. In one embodiment, the macrophage is a splenic macrophage.

The term “disease involving an antigen” refers to any disease in which an antigen is involved, for example, a disease characterized by presence of an antigen. The disease involving an antigen may be an infectious disease or cancer disease or simple cancer. As described above, the antigen may be a disease-associated antigen such as a tumor-associated antigen, viral antigen or bacterial antigen. In one embodiment, the disease involving an antigen is, a disease involving a cell expressing an antigen, preferably, on the surfaces of cells.

The term “cancer disease” or “cancer” refers to a pathological condition characterized by cell proliferation uncontrolled typically in an individual or mean this. Examples of cancer include, non-restrictively, carcinoma, lymphoma, blastoma, sarcoma, and leukemia. More specifically, examples of such a cancer include bone cancer, blood cancer, lung cancer, small cell lung cancer, non-small cell lung cancer, squamous cell carcinoma, adenocarcinoma, large cell lung cancer, liver cancer, pancreatic cancer, skin cancer, head and neck cancer, skin or intraocular melanoma, uterine cancer, ovarian cancer, rectal cancer, anal region cancer, gastric cancer, colorectal cancer, breast cancer, prostate cancer, endometrial cancer, sarcoma, pheochromocytoma adrenal tumor, testicular germ cell tumor, cervical cancer, carcinoma of sexual and reproductive organs, Hodgkin's disease, esophageal cancer, small intestinal cancer, endocrine cancer, thyroid cancer, parathyroid cancer, adrenal cancer, soft tissue sarcoma, bladder cancer, kidney cancer, renal cell carcinoma, renal pelvic carcinoma, central nervous system (CNS) neoplasm, neuroectodermal tumor, spinal tumor, glioma, meningioma and pituitary adenoma. According to the present specification, the term “cancer” also includes cancer metastasis.

A combination strategy in cancer treatment, may be appropriate due to a synergistic effect achieved, which may be considered higher than the effect of a monotherapy way. In one embodiment, the pharmaceutical composition is administered with an immunotherapeutic agent. As used herein, “immunotherapeutic agent” refers to any material that can be involved in activating a specific immune response and/or an immune effector function(s). The present specification considers a use of an antibody as an immunotherapeutic agent. Without meaning to connecting by theory, but the antibody can achieve a therapeutic effect on cancer cells through various mechanisms, including inducing apoptosis, blocking components of a signaling pathway, or inhibiting proliferation of tumor cells. In a specific embodiment, the antibody is a monoclonal antibody. The monoclonal antibody can induce apoptosis through antibody-dependent cell-mediated cytotoxicity (ADCC), or can induce direct cytotoxicity known as complement-dependent cytotoxicity (CDC). Non-restrictive examples of the anti-cancer antibody and potential antibody target (in parentheses) that can be used in combination with the present invention, include Abagovomab (CA-125), Abciximab (CD41), Adecatumumab (atumumab) (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD19), Brentuximab vedotin (CD30 TNFRSF8), Cantuzumab mertansin (mucin CanAg), Cantuzumab ravtansine (MUC1), Capromab pendetide (prostate carcinoma cell), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin aγβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (IL1β), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab

(OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (Fibronectin extra domain B), Rafivirumab (Rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL6), Tabalumab (BAFF), Tacatuzumab tetraxetan (α-fetoprotein), Taplitumomab paptox (CD 19), Tenatumomab (tenascin C), Teprotumumab (CD221), Ticilimumab (CTLA-4), Tigatuzumab (TRAIL-R2), TNX-650 (IL13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1 BB), Volociximab (integrin a5β1), Votumumab (tumor antigen CTAA 16.88), Zalutumumab (EGFR) and Zanolimumab (CD4).

In one example of the present invention, provided is a method for providing information to diagnose or determine a risk of metastasis of cancer. The method for providing information according to the present invention, may comprise determining that the subject with cancer has a high possibility of cancer metastasis, when Ser25 in the wild-type FoxM1 protein (SEQ ID NO: 1) is phosphorylated, or S25D or S25E mutation is comprised, in cancer cells isolated from a subject with cancer, or determining that the subject with cancer has a high possibility of cancer metastasis, when the 73th nucleic acid to the 75th nucleic acid in the wild-type FOXM1 gene (SEQ ID NO: 3) are substituted with 5′-GAT-3′, 5′-GAC-3′, 5′-GAA-3′, or 5′-GAG-3′ in cancer cells isolated from a subject with cancer, and thereby, the 25th amino acid of the protein expressed therefrom is substituted from Ser to Asp, or Glu.

In one example of the present invention, provided is a recombinant metastatic cancer cell expressing a polypeptide in which the 25th amino acid of SEQ ID NO: 1 is substituted from Ser to Asp, or Glu, and/or a recombinant metastatic cancer cell in which the 73th nucleic acid to the 75th nucleic acid in the nucleic acid sequence represented by SEQ ID NO: 3 are substituted with 5′-GAT-3′, 5′-GAC-3′, 5′-GAA-3′, or 5′-GAG-3′. 5′-GAT-3′, and 5′-GAC-3′ are codons encoding Asp, and 5′-GAA-3′, and 5′-GAG-3′ are those encoding Glu. The metastatic cancer cell has high migration, invasion, and/or proliferation, so it can be used for producing a cell for studying metastatic cancer, or a metastatic cancer in vivo model by inoculating it into an animal, and the like.

In the present invention, a pharmaceutical composition for treating cancer comprising shRNA specifically binding to FOXM1.

At first, through KEGG 2019 pathway analysis, a related pathway of invasive cells in which active PLK1 is expressed under cancer metastasis conditions of lung cancer cell A549. Pathways such as p53 signaling, DNA replication, cell aging, cell cycle, TGF-β signaling, and the like are involved (FIG. 11A).

The inventor of the present invention has confirmed that the increase of FoxM1 could be a biomarker in the cancer metastasis process by observing the increase in expression of FoxM1 simultaneously with mesenchymal cell markers, N-cadherin, vimentin, SNAI1, SNAI2 among EMT markers by TGF-β treatment.

For this, as a result of observing the increase of FoxM1 with mesenchymal cell markers (N-cadherin, vimentin, SNAI1, SNAI2) using immunoblotting in A549, NCI-H358, NCI-H460 cells which are non-small cell lung cancer cell lines treated with TGF-β for 48 hours, it was observed to be consistently increased in all the cells (FIG. 11B).

In addition, in order to confirm whether EMT is changed by expression of FoxM1, when the changes of EMT markers, which are transition factors, were observed by immunoblotting and RT-PCR in A549 cells, after introducing FoxM1 into pLVX-eRFP vector and introducing it, the protein and mRNA expression levels of the mesenchymal cell markers (E-cadherin, N-cadherin, Vimentin) were observed with an increase in FoxM1. It could be observed that the expression of E-cadherin was reduced, and the expression of N-cadherin and Vimentin was increased (FIGS. 12A, 12B).

Furthermore, in order to observe that the metastasis and invasion of cancer cells by FoxM1 are increased, a migration assay and an invasion assay using a transwell insert were performed (FIGS. 12C, 12D).

In order to observe metastasis of cancer cells in A549 cells in which the wild-type (WT) protein of FoxM1 is expressed, a cancer cell migration experiment using Transwell was conducted (FIG. 12C). As a result of the study, the wild-type experimental group of FoxM1 was increased by approximately 3 folds compared to the control group, and this shows the same pattern as the positive control, TGF-β treated cell group (5 ng/ml).

In addition, to examine the effect or promoting invasion in A549 cells in which the wild-type (WT) protein of FoxM1 is expressed, the invasion of cancer cells was to be observed using an invasion analysis using Matrigel (FIG. 12E). At first, in order to evaluate invasion, cells expressing each FoxM1 wild-type protein were aliquoted with a serum-free medium to the Matrigel insert, and a serum-containing medium was aliquoted into an experimental plate and cultured for 5 days. The invaded cancer cells were observed by staining them with crystal violet, and dissolved with DMSO, and then the absorbance was measured at a wavelength of 590 nm. As a result, it was observed that the invasion was increased about 20 folds compared to the control group in the lung cancer cell group expressing the wild-type protein of FoxM1. This showed the same pattern as the positive control group, TGF-β treatment cell group (5 ng/ml).

In addition, as a result of examining the expression of an immune evasion factor, CD274 in A549 cells in which the wild-type (WT) protein of FoxM1 is expressed through real-time polymerase chain reaction (real-time PCR), it could be confirmed that the expression was high about 2.3 folds in the A549 cells in which the wild-type (WT) protein of FoxM1 was expressed (FIG. 12E).

Through this, the present researcher could find that an increase of EMT markers was observed and the migration and invasion of cells were increased similarly to the TGF-β treated group, when FoxM1 was expressed.

In addition, in order to obtain whether to affect conversion of macrophages into tumor-associated macrophages (TAM) by FoxM1, by co-culturing THP-1 cells with A549 cells expressing FoxM1, a study on the increase and polarization of tumor-associated macrophage markers was performed using RT-PCR (FIG. 13).

Based on the result of the study showing that FoxM1 is involved in migration and polarization and the like of macrophages (BALLI, David et al., Oncogene (2012) 31.34:3875-3888; YANG, Yang et al., Diabetes Research and Clinical Practice (2022) 184:109121), to observe whether cancer cells expressing FoxM1 S25E affect polarization of macrophage cells, after co-culturing human macrophages, THP-1 cells, and A549 cells overexpressing the FoxM1 wild-type protein for 48 hours, M1 and M2 markers were observed. As a result, the expression of M1 markers, INOS, IL12B was not changed, but that of M2 markers, IL10, CD163, CD206, TGFB1, and VEGFA was increased all in the cells in which the FoxM1 wild-type protein was expressed (FIG. 13A). Among them, CD206, TGFB1, and VEGFA are known as markers of M2d-tumor associated macrophages (M2d-TAMs) (JAYASINGAM et al., Front. Oncol (2020) 9:1512). It could be observed that M2 inducing factors, IL4, IL6, IL10, VEGFA, and an immune evasion factor, CD274 were increased all in the A549 cells after co-culturing A549 cells expressing the FoxM1 wild-type protein and THP-1. This shows that the A549 cells expressing the FoxM1 wild-type protein polarizes THP-1 cells into M2d-TAMs.

Furthermore, as a result of measuring the amount of TFGB1, VEGFA proteins in a medium in which the A549 cells expressing the FoxM1 wild-type protein were cultured for 48 hours by ELISA, it was observed that it was significantly increased compared to the control group in the cells expressing the FoxM1 wild-type protein (FIG. 13C). As a result of analyzing the broad macrophage marker, CD68 and the tumor-associated macrophage marker, CD163 in lung tissue of mice by immunostaining, it was confirmed that the high expression of CD68, CD163 markers in the mouse lung tissue injected with the A549 cells in which the FoxM1 wild-type (WT) protein was expressed was observed (FIG. 13D). Through this, it can be seen that the increase and polarization of the TAM markers of THP-1 were increased when the cancer cells expressing FoxM1 were co-cultured with the THP-1 cells.

The inventor of the present invention has confirmed the inhibitory effect on the migration of cancer cells by FoxMl shRNA, a FoxM1 mRNA inhibitor (FIG. 14).

In order to inhibit the mRNA expression of FoxM1, pLKO-puro. 1-hFoxM1 plasmid was produced using pLKO-puro.1 vector to produce shRNA targeting the nucleotide sequences at positions 187-207 and 709-729 of the human FoxM1 mRNA sequence, respectively. The following sequences can be used as target sequences for production of human FoxM1 shRNA, and oligonucleotides with the following sequences can be used as primers for shRNA production. The access number for the human FoxM1 mRNA gene in Pubmed is U74613 and has 3326 bp.

#1 Target nucleotide positions 187-207
Nucleotide sequences
sense region:
5′-CATCAGAGGAGGAACCTAAGA-3′
anti-sense region:
5′-TCTTAGGTTCCTCCTCTGATG-3′
Primers for shRNA production
Forward primer
5′-ccgg-CATCAGAGGAGGAACCTAAGA-ctcgag-
TCTTAGGTTCCTCCTCTGATG-tttttg-3′
Reverse primer
5′-aattcaaaaa-CATCAGAGGAGGAACCTAAGA-ctcgag-
TCTTAGGTTCCTCCTCTGATG-3′
#2 Target nucleotide positions 709-729
Nucleotide sequences
sense region:
5′-AGCAAGAGATGGAGGAAAAGG-3′
anti-sense region:
5′-CCTTTTCCTCCATCTCTTGCT-3′
Primers for shRNA production
Forward primer
5′-ccgg-AGCAAGAGATGGAGGAAAAGG-ctcgag-
CCTTTTCCTCCATCTCTTGCT-tttttg-3′
Reverse primer
5′-aattcaaaaa-AGCAAGAGATGGAGGAAAAGG-ctcgag-
CCTTTTCCTCCATCTCTTGCT-3′

First, in order to confirm the inhibitory effect on FoxM1 expression by infecting the virus to A549, lung cancer cells, after purifying and concentrating each lentivirus for expressing FoxM1 shRNA produced, the level of mRNA expression and protein expression was observed (FIGS. 14A and 14B). As a result, it was observed that shRNA targeting the nucleotide sequence at positions 709-729 (target #2, 709-729 bp) had the most excellent effect on inhibiting FoxM1 expression (FIGS. 14A, 14B).

The inhibitory effect on the metastasis of cancer cells by the lentivirus for expressing FoxM1 shRNA selected by the above experimental results is provided.

For this, the lentivirus for expressing FoxM1 shRNA was infected to A549, lung cancer cells, and an experiment was conducted on the effect of inhibiting the migration of lung cancer cells (FIG. 14C).

In order to confirm the effect of the FoxM1 shRNA on metastasis of lung cancer cells by infecting the produced virus to A549, lung cancer cells, after purifying and concentrating the produced lentivirus for expressing FoxM1 shRNA, a migration assay of cancer cells was carried out for 72 hours (FIG. 14C). As a result of comparing the migration of cancer cells with the control group in 72 hours after creating a scratch, it was observed that the relative metastasis of the cells was reduced to about −25% or less at 72 hours in case that the expression of FoxM1 was inhibited with FoxMl shRNA in lung cancer cells (FIG. 14D).

It was observed that the cancer metastasis induced by treatment of TGF-β was significantly reduced by inhibition of FoxM1 mRNA expression with treatment of FoxM1 shRNA (#709), and CDH2, a mesenchymal marker, was significantly reduced (FIG. 14E).

In order to observe the level of apoptosis of cancer cells in the A549 cells by inhibition of expression of FoxM1 by treatment of FoxMl shRNA, a caspase-3 assay was conducted to measure the activity of caspase-3, a representative lyase of apoptosis (FIG. 14F). As a result, it was observed that the apoptosis was induced and increased by inhibition of expression of FoxM1 by treatment of FoxM1 shRNA, and in particular, it was observed that the FoxM1 shRNA targeting the 709-729 sequence significantly increased the activity of caspase-3 (FIG. 14F).

In addition, the present invention provides an inhibitory effect on conversion of monocytes in a tumor microenvironment, THP-1 into tumor-associated macrophages, caused by overexpression of the FoxM1 variant, by FoxM1 shRNA, a FoxM1 mRNA inhibiting material (FIG. 15).

As a result of infecting FoxM1 shRNA, a FoxM1 mRNA inhibiting material, to cells overexpressing the FoxM1 phosphorylated mutant, it could be observed that the FoxM1 expression was significantly reduced (FIG. 15A). As a result of examining the expression of M2 markers, CD163, CD206, VEGFA in THP-1 cells, by co-culturing upper cells and human mononuclear macrophages, THP-1 for 48 hours based on the above, it could be observed that the expression of FoxM1 was inhibited and the expression of upper factors was also reduced by FoxM1 shRNA (FIG. 15B).

In addition, as a result of inhibiting the FoxM1 expression by infecting FoxM1 shRNA, a FoxM1 mRNA inhibiting material, to cells overexpressing the FoxM1 phosphorylated mutant, it could be observed that the expression IFITM1 was also accordingly reduced (FIG. 15C).

Moreover, the present invention provides an inhibitory effect on metastasis of cancer cells by thiostepton, a FoxM1 inhibitor.

As in FIG. 16, in order to observe the effect on metastasis of lung cancer cells caused by overexpression of the FoxM1 variant using thiostepton, a FoxM1 inhibitor, the drug was treated for 48 hours.

As a result of the study, it could be observed that thiostepton reduced the expression of mesenchymal markers, CDH2, VIM, SANI1, and SNAI2 of lung cancer cells themselves (FIG. 16A). In addition, it could be observed that thiostepton reduced not only metastasis of cancer cells themselves but also the expression of IL6 and VEGFA, tumor-associated macrophage inducing factors, and the expression of CD274, an immune evasion factor was also reduced (FIG. 16B).

In addition, the present invention provides an anticancer therapeutic agent for metastatic cancer as well as primary cancer comprising a FoxM1 shRNA or FoxM1 inhibitor as an active ingredient.

In one example of the present invention, a pharmaceutical composition comprising at least one inhibitory nucleic acid capable of inhibiting expression or activity of protein expressed by a FOXM1 nucleic acid is disclosed. In one example of the present invention, for example, by administering a therapeutically effective dose of the inhibitory nucleic acid for inhibiting the expression or activity of protein encoded by the FOXM1 nucleic acid in tumor cells such as patient's cells, cancer patients can be treated. The inhibitory nucleic acid disclosed in the composition of the present invention is substantially complementary to the nucleic acid sequence of the target gene, FOXM1.

The present invention relates to a method for treating a patient diagnosed to have cancer or a disease characterized by expression of FOXM1, and the disease may be treated as symptoms related to overexpression of FOXM1 are alleviated by administering the inhibitory nucleic acid capable of inhibiting the expression or activity of protein encoded by the FOXM1 nucleic acid.

The pharmaceutically effective dose for inhibiting expression or activity of FOXM1 according to one example of the present invention may be determined by those skilled in the art considering the patient's age, body weight and response, and the like in the context of disease to be treated or prevented. In one example of the present invention, the effective dose means an amount that causes inhibition, prevention or treatment of cancer in a tissue, animal, human, or the like, of a compound, pharmaceutical composition, or drug capable of habiting expression or activity of FOXM1.

In one example of the present invention, the pharmaceutically effective dose of the compound capable of inhibiting expression or activity of FOXM1 can be delivered as a pharmaceutical composition. In one example, the pharmaceutical composition may be a product containing the compound inhibiting expression or activity of FOXM1, and the product may be a product comprising a specific component in a specific amount, or a product by a combination of specific components in a specific amount, directly or indirectly.

In one example, the inhibitory nucleic acid inhibiting expression or activity of FOXM1 may control expression of protein encoded by FOXM1, by binding to a part of the FOXM1 nucleic acid. The nucleic acid according to the present invention may comprise an exact or partial mismatch sequence in a target region. A number of nucleic acid molecules can control expression or activity of protein encoded by FOXM1. The nucleic acid molecules include antisense RNA, shRNA, siRNA, miRNA, RNA aptamer, DNA aptamer, and decoy RNA. Each nucleic acid molecule may be used for inhibiting expression or activity of FOXM1.

In one example, the inhibitory nucleic acid may be an siRNA which comprises about 15 to about 50 base pairs, for example, 21 to 25 base pairs, and comprises a double strand structure having the same or almost same nucleic acid sequence as an intracellular target gene or RNA. Antisense nucleic acid includes morpholinos, 2′-O-methyl polynucleotide, DNA, or RNA, or the like, but not limited thereto. RNA polymerase III-transcribed DNA contains a promoter such as U6 promoter. These DNAs produce small hairpin RNAs that can operate as siRNAs or linear RNAs that can operate as antisense RNAs in cells by being transcribed. The inhibitory nucleic acid inhibiting expression or activity of FOXM1 may comprise ribonucleotide, deoxyribonucleotide, synthetic nucleotide or any suitable combination that can inhibit a target RNA and/or gene. In addition, the form of the nucleic acid may be single-stranded, double-stranded, triple-stranded or quadruple-stranded.

The inhibitory nucleic acid may be chemically or biologically produced, or be expressed by a plasmid or recombinant viral vector.

The inhibitory nucleic acid according to the present invention may be a short hairpin RNA (shRNA). shRNA may be synthesized in vitro or be formed by being transcribed from an RNA polymerase III promoter in vivo. Those used for preparing such an shRNA and cleaving a gene in mammalian cells are described in Paddison, P. J., Caudy, A. A., Bernstein, E., Hannon, G. J., Conklin, D. S. (2002). Short hairpin RNAs (shRNAs) induce sequence-specific silencing in mammalian cells. Genes Dev, 16:948-58; McCaffrey, A. P., Meuse, L., Pham, T. T., Conklin, D. S., Hannon, G. J., Kay M. A. (2002). RNA interference in adult mice. Nature, 418:38-9; McManus, M. T., Petersen, C. P., Haines, B. B., Chen, J., Sharp P. A. (2002). Gene silencing using micro-RNA designed hairpins. RNA, 8:842-50; Yu, J.-Y., DeRuiter, S. L., Turner, D. L. (2002). RNA interference by expression of short-interfering RNAs and hairpin RNAs in mammalian cells. Proc Natl Acad Sci USA, 99:6047-52, and the like. This shRNA can continuously and stably inhibit a gene required to be engineered in a cell or animal. Those skilled in the art may understand that the shRNA is treated in a cell to produce an siRNA.

In one example, the shRNA comprises at least one sequence of SEQ ID NOs: 7 to 10. In one example, the shRNA comprises a sequence of SEQ ID NO: 7 or 8, and this may be used for inhibiting expression or activity of FOXM1 in vivo. In one example, the shRNA comprises a sequence of SEQ ID NO: 9 or 10, and this may be used for inhibiting expression or activity of FOXM1 in vivo.

The inhibitory nucleic acid may be designed and synthesized to form a duplex with a target RNA by comprising a non-complementary region (3 to 6 nucleotides long) between complementary regions of 15 to 30 nucleotide in length. The inhibitory nucleic acid capable of inhibiting expression or activity of the nucleic acid encoded by FOXM1 may be 18 to 100 nucleotides in length, and in one example, those skilled in the art may design to be modified into a mature form. For example, the mature miRNA may be 19 to 30 nucleotides, 21 to 25 nucleotides, or 21, 22, 23, 24 or 25 nucleotides in length, and herein, the miRNA precursor may have 70 to 100 nucleotides in length and have a hairpin structure.

The afore-mentioned inhibitory nucleic acid may be an RNA, DNA, or an oligomer or polymer of these two. This term may include not only naturally occurring nucleobases, sugars, and backbones, but also oligonucleotides having a non-natural moiety with functional similarity.

The inhibitory nucleic acid may comprise a sufficiently complementary nucleotide sequence to bind to FOXM1. In one example, the sufficient complementarity may be 12 to 25 nucleotides, 13 to 23 nucleotides, 14 to 23 nucleotides, or 15 to 23 nucleotides. The nucleic acid molecule of the present invention may be actually any length. The inhibitory nucleic acid that can inhibit expression or activity of protein encoding FOXM1 may be 20 to 100 nucleotides long, for example, 21, 22, 23, 24, 25, 26, 27, 28, 29, 30, 31, 32, 33, 34, 35, 36, 37 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 nucleotides long.

In one example, the inhibitory nucleic acid is considered to target FOXM1, when the stability of a target gene transcript under presence of the inhibitory nucleic acid of the present invention is reduced compared to the stability under absence, and/or the inhibitory nucleic acid has a sequence with homology of 90% or higher to the target transcript for a length of at least 17, at least 18, at least 19, at least 20, at least 21, at least 22, or at least 23, and/or the inhibitory nucleic acid binds to the target transcript under stringent conditions.

The inhibitory nucleic acid may be biologically produced using an expression vector.

The inhibitory nucleic acid of the present invention may be synthesized on a cell basis in vivo or be produced through chemical synthesis in vitro. The inhibitory nucleic acid that inhibits expression or activity of protein encoded by FOXM1 may be produced using chemical synthesis and/or enzymatic ligation reaction according to a method known in the art.

The inhibitory nucleic acid comprises a sufficiently complementary region for a target nucleic acid, and consists of a length sufficient so that the miRNA can form a double structure with the target nucleic acid. The inhibitory nucleic acid capable of inhibiting expression or activity of protein encoded by FOXM1 comprises a partially or completely complementary region for the target RNA. The inhibitory nucleic acid capable of inhibiting expression or activity of protein encoded by FOXM1 and the target sequence do not have to be completely complementary, but they should be corresponding enough to inhibit the expression of the target gene.

The inhibitory nucleic acid may be synthesized by being modified to have desired properties. For example, the modification may be performed to improve stability, or increase the probability of thermodynamic hybridization with a target nucleic acid, or increase targetability to a specific tissue or cell type, or increase permeability to cells. The modification may be performed to increase sequence specificity and reduce non-specific binding.

The inhibitory nucleic acid molecule may have the substantially same nucleotide sequence to a part of the nucleic acid encoding FOXM1. As described above, the substantially same single-stranded oligonucleotide to at least a part of the nucleic acid encoding FOXM1 may be administered to a patient having cancer or at risk of having cancer.

The inhibitory nucleic acid may be delivered to cells as a naked plasmid or an expression vector comprising a viral vector comprising a DNA virus and an RNA virus including adenoviruses, lentiviruses, alphaviruses, and adeno-associated viruses, or in any of a variety of forms comprising other DNA formulated in liposomes. The required amount of nucleic acid may be determined depending on the delivery formulation or whether the nucleic acid used is a DNA or RNA by those skilled in the art.

The inhibitory nucleic acid capable of inhibiting expression or activity of protein encoding FOXM1 may be expressed from a transcription unit inserted into a DNA or RNA vector. The recombinant vector may be a DNA plasmid or viral vector. The viral vector suitable for producing the inhibitory nucleic acid capable of inhibiting expression or activity of protein encoding FOXM1, may be designed based on for example, adeno-associated viruses, retroviruses, adenoviruses, or alphaviruses, but not limited thereto. The recombinant vector that can express the inhibitory nucleic acid capable of inhibiting expression or activity of protein encoding FOXM1 may be delivered according to the above method, and may be sustained in a target cell, or may provide only transient expression of the nucleic acid molecule. Such a vector may be repeatedly administered as needed. Once expressed, the inhibitory nucleic acid interacts with the target RNA to inhibit the miRNA activity. Including papovaviruses, a number of viruses such as for example, SV40, adenoviruses, vaccinia viruses, adeno-associated viruses, herpesviruses, and avian, rodent, and human origin retroviruses may be used in the present invention. In one example, a lentiviral vector may be also used in the present invention. In one example, the lentiviral vector may be a doxycycline-inducible lentiviral vector engineered to express at least one shRNA against FOXM1.

Delivery of a vector expressing the inhibitory nucleic acid may be administered systemically such as intravenous or intramuscular injection, or locally to a target organ or tissue, or the like.

Since nucleic acid hydrolase cleaving phosphodiester bonds in DNA are expressed in most cells, unmodified DNA such as inhibitory oligonucleotides, is generally modified mostly so as not to be degraded. In addition, most targets of antisense exist in cells, so it should be considered that the nucleic acid enters cells. The inhibitory nucleic acid having a nucleotide modified so as not to be degraded is preferred for clinical use. Furthermore, in order to increase selectivity for targeting a specific cell or to enable it to pass through cell membranes, other molecules may be fused.

Through chemical modification of the inhibitory nucleic acid, physiological activity can be significantly improved. By modifying antisense nucleic acids with phosphorothioate, the binding ability to cell surface proteins can be improved. Cell delivery can be improved by fusing a positively charged arginine-rich peptide to PMO-modified antisense nucleic acids.

An intracellular delivery system comprises an antisense nucleic acid fusion and a cationic lipid carrier, a carrier molecule binding to a cell-specific receptor, a cyclodextrin, a dendrimer, a microparticle, and a macromolecule. This delivery system can enhance the intracellular delivery efficiency by protecting antisense nucleic acids from nucleic acid degradation and/or promoting absorptive endocytosis.

The macromolecule includes a cell-penetrating peptide (CPP), a cationic short peptide sequence fused to antisense nucleic acids through disulfide bonds. The CPP generally used includes penetratin, HIV TAT peptide 48-60, and transportan. In addition, by combining dioleylphosphatidylethanolamine to a liposome delivery system, endosome membranes can be destabilized and the release of antisense nucleic acids can be promoted after endocytosis.

In order to enhance the physiological activity after administration, encapsulation in an inert, biodegradable albumin polymer matrix can be conducted, and it has been reported that the physiological activity of 9% to 70% can be enhanced through this. In addition, it has been reported that pharmacokinetic indexes such as half-life and volume of distribution can be also increased through microencapsulation.

Several nanoparticle-based siRNA deliver systems have been approved by FDA and have been introduced. Currently, all siRNA delivery systems formulated with nanoparticles in clinical trials for cancer treatment are based on polymers or liposomes.

Nanoparticles fused to target ligands for effective siRNA delivery have an increased probability of binding to tumor surface receptors, but due to this process, the overall size of the nanoparticles increases. When nanoparticles are coated with PEG, absorption by RES is reduced, and consequently, the circulating half-life is increased, but PEG molecules interfere with spatially selective binding, and therefore, target specificity is reduced. Thus, it is important to select an appropriate cell specific target moiety and design a stable and effective nanoparticle delivery system. The delivery system based on various nanoparticles such as cationic lipids, polymers, dendrimers, and inorganic nanoparticles is known to effectively deliver siRNA in vitro and in vivo.

Antisense nucleic acids can be delivered systemically, such as oral administration, or through local administration to tumors, by being formulated in saline solution with chemical modifications so as to be absorbed. Their phosphorothioate backbone binds to serum proteins and slows secretion by the kidney. Aromatic nucleobases interact with other hydrophobic molecules in serum and on the cell surface. Many types of cells express surface receptors that absorb oligonucleotide in vivo, and they may often disappear in cultured cells, and this demonstrates why lipids seem to be more important for ASO (allele-specific oligonucleotide) delivery during culturing than in vivo.

Delivery of double-stranded RNA is more difficult than single-stranded nucleic acid. In siRNA, all aromatic nucleobases are positioned on the inside, and only a significant amount of hydrated phosphates are located on the outside. The hydrated surface little interacts with the cell surface, and is rapidly released in the urine. Therefore, researchers have performed many studies to develop a carrier of siRNA. The main technique for delivering siRNA, is to mix RNA with cationic and neutral lipids, although desired results have been obtained with peptide transduction domains and cationic polymers. The circulating half-life of the particles can be extended, by comprising PEGylated lipids in the formulation. By fusing cholesterol to one strand of siRNA, knockdown has been effectively induced in the liver of mice, but there was a disadvantage of the required amount of active ingredient lower several times than that of common lipid-based formulations.

One way for optimization of single-stranded DNA or RNA is using chemical modifications to increase resistance to nucleases, such as introducing a phosphorothioate (PS) bond at a phosphodiester binding site. Such modifications greatly improve stability against digestion by nucleases. The PS bond also enhances binding ability to serum proteins in vivo, increases the half-life, and makes active ingredients be delivered to tissue better. The ASO comprising only the PS modification can make the antisense effect in cells, but the efficacy is not always high, and the results are not usual enough to be reliable.

Chemical modifications can also enhance efficacy and selectivity by increasing the binding affinity of nucleic acid to a complementary sequence. Widely used modifications are 2′-O-methyl (2′-O-Me), 2′-fluoro (2′-F), and 2′-O-methoxyethyl (2′-MOE) RNA. In addition, higher affinity can be obtained by modifying oligonucleotide with LNA (locked nucleic acid) containing a methylene bridge between 2′ and 4′ positions of ribose. The bridge is an ideal structure that can achieve binding to a complementary sequence and high affinity, and fixes a ribose ring. Related BNA (bridged nucleic acid) compounds have been developed, and these desired properties are shared. Their high affinity made it possible to develop oligonucleotide much shorter than a possible length conventionally thought. A synthesis method to introduce 2′-O-Me, 2′-MOE, 2′-F, or LNA into oligonucleotide are compatible with DNA or RNA synthesis, so that chimeras with DNA or RNA can be easily obtained. This compatibility allows chemically modified oligonucleotide to be fine-tuned for a specific use.

Over the past decade, double-stranded siRNA has been widely utilized as a means to inhibit gene expression. When double-stranded RNA enters the cell, it binds to the protein mechanism of RISC. The synthesized RNA used to delete genes is generally a double strand of 19 to 22 bp. This length is sufficient to form a stable double strand to be recognized by RISC, yet short enough to avoid a strong interferon response induced by a double strand with a length of 30 pb or more.

Since the first paper on gene deletion in mammalian cells was published in 2001, siRNA has been a subject of thousands of experimental studies to test its function. While antisense oligonucleotides continue to be used for gene deletion, it became the preferred method of deletion in many laboratories, due to the strong properties of siRNA and the relative ease identification method of active siRNA.

In cultured cells, unmodified double-stranded RNA is surprisingly stable, and chemically modified siRNA is generally not essential for defects in gene expression. However, in vivo, unmodified siRNA does not have high activity, and its properties can be significantly improved through chemical modification. Chemically modified siRNAs can show improved nuclease stability and increased duration of activity. Unmodified RNA is also rapidly removed, and chemical modification, complex formation with carriers, and localized delivery to disease targets may help achieve improved results in vivo.

Oligonucleotides used in the present invention can be produced through a commonly known technique, for example, solid phase synthesis. The equipment used in this synthesis method is commercially available, and other means may also be added for synthesis.

Therapeutic administration of inhibitory nucleic acids to cells to inhibit the expression or activity of the protein encoded by FOXM1 nucleic acid includes administering any nucleic acid known to those skilled in the art. In order to treat cancer, it can be delivered via oral administration or injection, or both of them. Nucleic acid molecules can be delivered to cells through various methods known in the art, and for example, iontophoresis or combining to other carriers such as hydrogels, cyclodextrins, biodegradable nanocapsules, and bioconjugate microsphere, or encapsulation into liposomes by a proteinaceous vector may be used.

In a specific embodiment, the inhibitory nucleic acid to inhibit the expression or activity of the protein encoded by the FOXM1 nucleic acid may be administered to a cell organelle, cell, tissue, tumor, or organism by subcutaneous, intradermal, intramuscular, intravenous, intraperitoneal injection, and the like.

The above inhibitory nucleic acid or other active ingredient may be added to a pharmaceutical composition suitable for administration. For example, the pharmaceutical composition includes one or more inhibitory nucleic acids capable of reducing the expression or activity of the protein encoded by the FOXM1 nucleic acid and a pharmaceutically acceptable carrier.

The inhibitory nucleic acid may be provided as a sustained-release composition. Immediate release or sustained release compositions may be determined depending on the condition of the patient to be treated. If the patient's condition is an acute or hyperacute disease, the treatment should use an immediate-release type, whereas in the case of preventive or long-term treatment, a sustained-release composition is suitable.

The anticancer agent comprising the FoxM1 shRNA or FoxM1 inhibitor or FoxM1 protein comprising a point mutation of the present invention as an active ingredient may be used for prevention and treatment of especially, various carcinomas with xcessive expression of FoxM1, various kinds of solid cancers such as lung cancer (Wang, I-Ching et al. PLOS One 4.8 (2009): e6609), colorectal cancer (Yoshida, Yuichi et al., Gastroenterology 132.4 (2007) 1420-1431), prostatic cancer (Kalin, Tanya V. et al., Cancer research 66.3 (2006) 1712-1720), breast cancer (Millour, Julie and E. W. Lam. Breast Cancer Research 12.1 (2010) 1-1) and brain cancer (Liu, Mingguang et al., Cancer research 66.7 (2006) 3593-3602), and the like, and leukemia, and the like (Nakamura, Satoki et al., Carcinogenesis 31.11 (2010): 2012-2021), and in addition, it may be also used in prevention and treatment of metastatic solid cancers (Li, Lijun et al., Oncotarget 8.19 (2017): 32298).

Mention of literature and studies referenced herein is not intended as an admission that any afore-mentioned content is relevant to prior art. All mentions to the contents of these documents are based on information available to the applicant and are not considered as any admission as to the accuracy of the contents of these documents.

The following description is provided to enable those skilled in the art to make and use various embodiments. Descriptions of specific devices, techniques and uses are provided as examples only. Various modifications to the examples described herein will be easily apparent to those skilled in the art, and the general principles defined herein may be applied to other examples and uses without departing from the spirit and scope of the various embodiments. Accordingly, the various embodiments are not intended to be limited to the examples described and shown herein, but are intended to accord with the scope consistent to the claims.

### EXAMPLES

### Example 1

### Example 1.1

### Clinical Relevance of FoxM1 and Active Form of PLK1 in Metastatic Lung Cancer Cells and the Expression Analysis of FoxM1 and Active Form of PLK1 Under Conditions of TGF-β-Induced Cancer Metastasis

In a recent study (Shin S. B. et al., Oncogene 39 (2020) 767-785), changes in ECM-adhesion-related genes were observed during the metastasis process induced by PLK1 in non-small cell lung cancer cells (FIG. 1A). In order to analyze the correlation between FoxM1 and PLK1 in non-small cell lung cancer and examine its clinical significance, the inventors analyzed the correlation between the mRNA expression of FoxM1 and PLK1 in lung adenocarcinoma patients using big data registered in cBioPortal and analyzed Spearman and Pearson's correlation coefficients. As a result, it was confirmed that there was a positive correlation in the expression of the two factors within a significant range (FIG. 1A). In addition, the correlation was analyzed with other cell cycle-related factors, and the expressions of FoxM1 and MKI67 were analyzed as factors showing a high correlation coefficient with the PLK1 expression (FIG. 1B, 1C).

Next, in order to confirm the clinical relevance of FOXM1 and PLK1 in non-small cell lung cancer patients, the overall survival rate of patients according to the expression levels of FOXM1 and PLK1 was confirmed through big data analysis (FIGS. 1D, E, F). It could be demonstrated that the survival rate of patients with high expression of PLK1 and FOXM1 in non-small cell lung cancer patients was significantly lower than that of patients with low expression of PLK1 and FOXM1 (FIG. 1D). Non-small cell lung cancer is divided into adenocarcinoma and squamous lung cell carcinoma, and based on the results of analyzing them separately, it could be demonstrated that in adenocarcinoma patients, the survival rate of patients with high expression of PLK1 and FOXM1 was significantly lower than that of patients with low expression of PLK1 and FOXM1 (FIG. 1E). In particular, based on the results of reanalyzing patients with stage 3-4 lung adenocarcinoma in which metastases were observed, it could be demonstrated that the survival rate of patients with high PLK1 and FOXM1 expression was significantly lower than that of patients with low PLK1 and FOXM1 expression (FIG. 1F). Additionally, the results of analyzing the expression level of FoxM1 and PLK1 in lung adenocarcinoma patients by stage using a heatmap showed that PLK1 and FOXM1 expressions were higher in cancer patients than in normal tissues, and in stage 2-4 tissues than in stage 1 (FIG. 1G).

### Example 1.2

### Identification of Novel Phosphorylation Sites and Phosphorylation of FoxM1 by Active Form of PLK1 in TGF-β-Induced Metastatic Lung Cancer Cells

In the clinical data from the analysis of expression of FoxM1 and PLK1, survival rate, and expression according to cancer stage in patients with non-small cell lung cancer, it was observed that the expression of FoxM1 and PLK1 was higher in metastatic cancer, which may act as a cause of the lower survival rate. Therefore, the present inventors tried to observe the function of FoxM1 and PLK1 in a metastatic cancer model.

For this purpose, the expression changes of PLK1 and FoxM1 and the activation of PLK1 in the process of inducing epithelial-mesenchymal transition (EMT), the metastatic cell model, by treating with TGF-β, were observed. After inducing EMT by treating non-small cell lung cancer cell lines, A549, NCI-H358, and NCI-H460 with TGF-β, the mRNA and protein expression levels were analyzed.

In the non-small cell lung cancer cell lines A549, NCI-H358, and NCI-H460 treatedwith 5 ng/ml of TGF-β, an increase in mRNA expression levels of CDH2, SNAI1, and SNAI2 and a decrease in mRNA expression levels of the epithelial marker CDH1 were observed. Under these conditions, an increase in mRNA expression levels of PLK1 and FOXM1 could be observed compared to the control group (FIG. 2A, 2B, 2C). In addition, the protein amounts of vimentin, PLK1, E-cadherin, and N-cadherin showed the same pattern as the mRNA expression levels, and in particular, it could be observed that the amount of the phosphorylated protein at T210, which is the active form of PLK1, was highly increased in TGF-β-treated group compared to the control group (FIG. 2D). The relative change in the protein amount is shown as a graph (FIG. 2D, right panel).

To analyze the correlation between PLK1 and FoxM1, and to investigate whether the phosphorylation of FoxM1 depends on EMT, the phosphorylation levels of p-FoxM1, p-PLK1, and p-TCTP were analyzed by immunoprecipitation and immunoblotting using gel retardation and anti-phosphorylation antibodies after treating with phosphatase (CIP) to A549 and NCI-H460 treated with TGF-β. The results showed that CIP treatment reduced the mobility of PLK1, TCTP, and FoxM1 bands that were shifted upward by TGF-β treatment. In addition, based on the results using anti-phosphorylation antibodies, it could be found that the levels of p-FoxM1 Ser, p-PLK1 T210, and p-TCTP S46 were decreased by treatment with the phosphatase (FIG. 2E, 2F). It could be found that FOXM1 and PLK1 were phosphorylated during EMT induced by TGF-β treatment.

Therefore, FoxM1 and PLK1 were highly expressed in an environment where non-small cell lung cancer metastasis was induced, and an increase in PLK1 activity was correlated with FoxM1 phosphorylation. It could be found that there was a correlation between the phosphorylation of these factors and cancer metastasis.

### Example 1.3

### Establishment of a Lentiviral System for Expression of Phosphorylation and Non-Phosphorylated Point Mutants of FoxM1

To establish a lentivirus system for expression of phosphorylation-and non-phosphorylation-genes of FoxM1, the present inventors cut pLVX-TRE3G-eRFP with NotI and EcoRI and amplified FoxMl wildtype (WT) plasmid by PCR using primers, 5′-ACGGGGCCCATGAAAACTAGCCCCCGTCG-3′ (forward primer) and 5′-ACGGGAATTCCTACTGTAGCTCAGGAATAA-3′ (reverse primer). After cutting it with NotI and EcoRI, it was subcloned into the vector pLVX-TRE3G-eRFP. To establish the lentivirus system, HEK293 cells were transfected with pCMV-VSV.G and pCMV-Δ8.2, along with pLVX-TRE3G-eRFP-Target or pLVX-Tet3G DNA to express lentivirus, and the viruses were harvested and used in cancer cells. After transfection, the virus culture was collected at 12-hour intervals for up to 72 hours, filtered through a 0.2 mm filter, and centrifuged at 18,000 rpm, 4° C. for 90 minutes. The supernatant was discarded, and the viruses were harvested in TNE buffer, stored at 4° C., and used for cancer cell infection from the next day.

### Example 1.4

### Evaluation of Epithelial-Mesenchymal Transition Effect After Selection of Cells Expressing Active and Inactive FoxM1 Genes Using the Lentiviruses

To examine the effect of FoxM1 protein containing point mutations on regulating epithelial-mesenchymal transition in cancer cells, cancer cells were cultured as follows to infect lung cancer cells with lentiviruses expressing phosphorylation and non-phosphorylated point mutants of FoxM1.

To establish a stabilized cell line expressing WT, phosphorylation, and non-phosphorylated point mutants of FoxM1 in lung cancer cells, A549 cells were first infected with pLVX-Tet3G expressing lentivirus, and the infected cells were selected by treating with G418 for 5 days. The selected A549Tet3G cells were infected with lentiviruses expressing WT and phosphorylated point mutants (S25E, S361E, S715E) and non-phosphorylated point mutants (S25A, S361A, S715A) of FoxM1, and then treated with puromycin for 48 hours to establish a stabilized cell line. The established cells were treated with 2 μg/ml of doxycycline to induce expression of WT, phosphorylation, and non-phosphorylated point mutants of FoxM1, and the mRNA and protein expression levels of each FoxM1 point mutant were determined to confirm whether they were well expressed. As shown in FIG. 4A and FIG. 4B, it was confirmed that the WT and each point mutant form of FoxM1 were well expressed through mRNA and protein expression levels. It was observed that the expression levels of N-cadherin and vimentin were higher, and the expression levels of E-cadherin were lower in lung cancer cells expressing the phosphorylated form of FoxM1, specifically, phosphorylated at the phosphorylation site S25, compared to the cells expressing the non-phosphorylated form of FoxM1. In addition, when it was examined whether the expression of these phosphorylation and non-phosphorylated point mutants of FoxM1 affected cell proliferation, it was observed that it had no significant effect on cell proliferation (FIG. 4C).

These results suggested that the phosphorylated point mutant (S25E) of the FoxM1 S25 residue had an effect of promoting epithelial-mesenchymal transition.

### Example 1.5

### Evaluation of Promoting and Suppressing Metastasis Induced by Proteins Containing Phosphorylation and Non-Phosphorylation Point Mutations of FoxM1

To examine the effects of FoxM1 WT, phosphorylation, and non-phosphorylated point mutants on the motility of cancer cells, the migration assay was performed in A549 lung cancer cells expressing mutant proteins.

Specifically, 5×10⁴ cells of lung cancer cells A549 expressing each of WT, phosphorylation, and non-phosphorylated point mutant of FoxM1 were seeded in an 8.0 μm, 24 well insert, and the inserts were placed in a 24-well plate after medium with 10% fetal bovine serum (FBS) was dispensed in the 24-well plate. For the positive control, 0.5 ml of RPMI1640 (10% FBS) containing 5 ng/ml of TGF-β was used. After 72 hours from cell seeding, 500 μl of 4% paraformaldehyde was dispensed, washed three times with 1XPBS, and stained with 0.05% crystal violet solution for 5 minutes. After 5 minutes, the cells were washed five times with 1XPBS, and the staining intensity was measured using the Odyssey infrared imaging system. When the staining intensity of the control was set to 1, the relative staining intensity of each experimental group was calculated and displayed on a graph.

As a result of the study, the staining intensity value was increased up to 6 times in the experimental group expressing the phosphorylated point mutant protein of FoxM1, compared to the control group, which is similar to or higher than the TGF-β-treated positive control group, and it could be observed that the staining intensity was relatively lower in the non-phosphorylated point mutant experimental group compared to in the FoxM1 phosphorylation mutant (FIG. 4D).

However, it could be observed that the S361 and S715 phosphorylation mutants of FoxM1 did not affect cancer cell motility. From these results, it could be found that the S25 phosphorylated point mutant of FoxM1 promoted the motility of lung cancer cells, while the S25 non-phosphorylated point mutant of FoxM1 effectively suppressed the motility of lung cancer cells.

### Example 1.6

### Evaluation of Promoting and Suppressing Invasion Induced by Proteins Containing Phosphorylation and Non-Phosphorylation Point Mutations of FoxM1

To observe the effects of the WT, phosphorylation, and non-phosphorylated point mutants of FoxM1 proteins on the invasion of cancer cells, an invasion assay using Matrigel was performed in lung cancer cells A549 expressing these mutants.

Specifically, after completely dissolving Matrigel at 4° C. for 16-20 hours, Matrigel was diluted with cold serum-free RPMI 1640 (4° C.) to 1 mg/ml. 100 μl of Matrigel mixture (1 mg/ml) was added to an 8.0 mm 24-well insert and solidified in a 37° C. incubator for 12-20 hours. 1×10⁵ cells/well of lung cancer cells A549 expressing each WT, phosphorylation, and non-phosphorylated point mutant FoxM1 proteins were diluted in serum-free RPMI 1640 (36° C.) and dispensed onto the insert. 0.5 ml/well of warm RPMI 1640 (10% FBS) at 36° C. was dispensed to the insert. For the positive control group, 0.5 ml of RPMI 1640 (10% FBS) containing 5 ng/ml TGF-β at 36°° C. was used. The medium was changed once every 3 days, and the degree of invasion was observed. On the 7th day, when sufficient cancer cell invasion was observed, the medium was removed, washed with 1X PBS, and the cells inside the insert were scraped with a cotton swab and washed with 1X PBS to remove any remaining cells and Matrigel inside the insert. 500 μl of 4% paraformaldehyde was dispensed into the 24 wells with the outer surface of the insert, incubated at room temperature for 5 minutes, washed three times with 1X PBS, and stained with 0.05% crystal violet solution for 5 minutes. After 5 minutes, it was washed five times with 1X PBS and dissolved in DMSO. The degree of staining was determined by measuring the absorbance at 590 nm. When the absorbance of the control group was set to 1, the relative absorbance of each experimental group was calculated and displayed on a graph.

As a result of the study, in the experimental group expressing the FoxM1 phosphorylated point mutant protein, the absorbance value increased by nearly 40 times compared to the control group, which was similar to or higher than the positive control, TGF-β treatment group, and relatively, the FoxM1 non-phosphorylated point mutant experimental group could be observed to have a lower relative absorbance (FIG. 4E). Therefore, it could be found that the non-phosphorylated point mutant of the FoxM1 S25 residue was effective in inhibiting the motility and invasion of lung cancer cells.

### Example 1.7

### Evaluation of the Effects of Proteins Containing Phosphorylation Point Mutations and Simultaneous Triple-Point Mutations of FoxM1 on the Motility Efficiency

To observe the effects of phosphorylation point mutations on the motility of cancer cells, a wound healing assay was performed in lung cancer cells A549 expressing FoxM1 wild type (WT), phosphorylated point mutants S25E, S361E, and S715E, and a simultaneous triple-point mutation EEE protein.

Specifically, cells expressing the gene of FoxM1 point mutant by the lentivirus system were seeded into 6-well plates at 1×10⁵ cells/well each. 24 hours after cell seeding, scratches were made at regular intervals using a 200 μl-pipette tip. In addition, A549 cells treated with mock as a negative control (Mock) and 5 ng/ml of TGF-β as a positive control were used. It was observed at intervals of 0 h, 24 h, 48 h, and 72 h, and the healing distance was measured (FIG. 4F). In addition, the distance in the control group at 72 h was set to 0%, and the relative distance was represented as a bar graph (FIG. 4G).

As a result of the study, it could be observed that the cell group expressing the phosphorylated point mutant (S25E) of the S25 residue in FoxM1 showed a significant increase in motility compared to the control group, and when the distance in the control group at 72 h was set to 0%, the distance in S25E was 43%. This showed the same increase pattern as the TGF-β-treated positive control (5 ng/ml), which was 51%. The motility of cancer cells expressing the phosphorylated point mutant (S361E) and the phosphorylated point mutant (S717E) protein was observed to be 0.7% and 1.6%, respectively, at 72 h compared to the control group, showing no significant difference. In addition, the motility of the simultaneous triple-point mutant (EEE), which was 15% at 72 h, could be observed to be lower than that of the point mutant (S25E) but was increased compared to the control group. Therefore, it could be found that in the cell group expressing the phosphorylated point mutant (S25E) of FoxM1 S25 residue, the motility of cancer cells increased, while the motility did not increase significantly in the cells expressing two other phosphorylated point mutants, S361E and S715E. From these results, it could be found that the phosphorylated point mutant of FoxM1 S25 residue could effectively promote the motility of lung cancer cells.

### Example 1.8

### Evaluation of Metastatic Potential and Tumorigenic Potential of Cancer Cells by Proteins Containing Phosphorylation and Non-Phosphorylation Point Mutations of FoxM1 in Animal Models

To observe the tumorigenic potential of cancer cells in animal models, the present inventors aimed to evaluate the tumorigenicity-promoting and-suppressing effects of cancer cells by using cancer cells expressing phosphorylation and non-phosphorylated point mutants of FoxM1 S25 residue.

Specifically, A549 cancer cells (2×10⁶ cells) stably expressing FoxM1 protein containing point mutations were diluted in PBS and injected into the tail vein of mice. The mice were raised for 8 weeks, and then laparotomy was performed to observe cancer cell metastasis and tumorigenesis in organs. For the comparison, the cancer cell metastasis and tumorigenesis were observed in the control group (Mock; the group treated with lentivirus in which the target gene is not expressed), the group administered with A549 lung cancer cell line expressing FoxM1 protein (WT), the group administered with A549 lung cancer cell line expressing the phosphorylated point mutant of FoxM1 protein (S25E), and the group administered with A549 lung cancer cell line expressing the non-phosphorylated point mutant of FoxM1 protein (S25A). The experiment was performed on 5 mice for each experimental group, and the frequency of tumor formation, which is metastatic cancer in the lung, was measured and displayed in a graph (FIG. 5).

As shown in FIG. 5, in the experimental group administered with A549 cells expressing FoxM1 protein containing the phosphorylation point mutation, relatively high tumorigenicity was observed, compared to the control group (Mock) and FoxM1 WT. Among the FoxM1 phosphorylated point mutants, the highest tumorigenicity could be demonstrated in the S325E experimental group when observing the number of tumors generated. On the other hand, the tumorigenicity was observed to be relatively low in the experimental group administered with A549 cells expressing FoxM1 protein containing the non-phosphorylation point mutations (FIG. 5A). Through H&E (Haematoxylin and eosin) and Ki67 staining, it could be found that the degree of cancer cell proliferation was high in the FoxM1 phosphorylated point mutant experimental group (FIG. 5B, 5C). This could confirm that cancer cells expressing the phosphorylated point mutant of FoxM1 promoted cancer metastasis and tumorigenicity, while conversely, the non-phosphorylated point mutant suppressed cancer metastasis and tumorigenicity in the animal models.

Next, when the lung tissues were lysed and the protein levels of epithelial-mesenchymal transition markers were measured, the protein levels of the mesenchymal marker N-cadherin increased in the FoxM1 phosphorylated point mutant experimental group, while the levels of E-cadherin decreased. In addition, the expression of PD-L1, which was known to be involved in immune evasion and to enhance the tumorigenic potential of cancer cells, was also examined in each experimental group. The expression of PD-L1 was found to be elevated in the experimental group expressing the phosphorylated point mutant of FoxM1 (FIG. 5D). Moreover, the consistent results were confirmed not only at the protein levels but also at the mRNA levels (FIG. 5E). Therefore, it was suggested that the phosphorylation of FoxM1 enhanced epithelial-mesenchymal transition, metastasis, and tumorigenic potential. On the other hand, the non-phosphorylated point mutant of the present invention, specifically, S25A, the experimental group, was more effective in reducing the metastasis and suppressing the tumorigenic potential in cancer cells compared to the FoxM1 WT experimental group.

Furthermore, to observe the degree of apoptosis of cancer cells in A549 cells expressing each of the phosphorylation and non-phosphorylated point mutants of FoxM1, a caspase-3 assay was performed to measure the activity of the caspase-3 enzyme (FIG. 5F). A549 cells expressing each protein containing the phosphorylation and non-phosphorylation point mutation of FoxM1were administered with 3 μg/ml of doxycycline for 48 hours to induce over-expression of each mutant. The cells were lysed and reacted with the fluorescence substrate of caspase-3 (Ac-DEVD-AMC) for 1 hour, and then the fluorescence value (Ex. 380 nm/Emi. 430 nm) was measured using an electron spectrometer (SpectraMax M4 system). The measured fluorescence value indicated the activity of caspase-3, and the relative measurement value in each experimental group was calculated and depicted by a graph, when the measured fluorescence value of the control group set to 1. As a result, the highest activity of caspase-3 was measured in the cells over-expressing the non-phosphorylated point mutant of FoxM1, and conversely, the lowest activity of caspase-3 was measured in cells over-expressing the phosphorylated point mutant of FoxM1. From these results, it could be found that over-expression of the non-phosphorylated point mutant of FoxM1 induced and increased apoptosis.

### Example 1.9

### Evaluation of Macrophage Polarization Potential by Proteins Containing the Phosphorylation and Non-Phosphorylation Point Mutations of FoxM1 (FIG. 6)

In the animal tissues, it was confirmed that both PD-L1 protein expression and CD274 mRNA expression were increased in cancer cells expressing FoxM1 (FIG. 5D, 5E). Based on these results, we introduced the possibility that tumor immune evasion may occur in the FoxM1 phosphorylated point mutant and researched the effects on the polarization of immune cells in the tumor microenvironment that may affect the immune evasion of cancers. From the results described above, we investigated whether FoxM1 phosphorylated point mutant (S25E) affects the polarization of macrophages in the tumor microenvironment. After co-culturing human monocyte

THP-1 cells and the cell groups over-expressing FoxM1 point mutants, M1 and M2 markers were examined in THP-1 cells. After co-culturing of S25E-expressing cancer cells (4×10⁴ cells/ml) and THP-1 cells (1.2×10⁵ cells/ml) for 48 h, it was observed that in THP-1 cells, the mRNA expression of M1 markers INOS and IL12B did not change, but the mRNA expression of M2 markers IL10, CD163, Cd206, TGFB1, and VEGFA increased all by 2 folds or more in S25E (FIG. 6A). Among them, CD206, TGFB1, and VEGFA were known as markers of M2d-tumor-associated macrophages (M2d-TAM) (JAYASINGAM, Sharmilla Devi, et al., Frontiers in Oncology (2020) 9:1512.), and high expression of markers of M2d-tumor-associated macrophages was observed in THP-1 cells co-cultured with A549 cells expressing FoxM1 S25E. It could be found that THP-1 cells were polarized to M2d-TAM by expression of the FoxM1 phosphorylated point mutant.

In addition, after co-culturing of human monocyte THP-1 cells and cell groups over-expressing FoxM1 point mutations for 48 hours under the conditions where the previous experiments were performed, the mRNA expression levels of M2d-inducing factors IL4, IL6, IL10, and VEGFA in A549 cells expressing FoxM1 S25E were analyzed. As a result, these factors were increased by 1.8 folds, 1.8 folds, 2.2 folds, 2.5 folds, and 2.5 folds, respectively, and in A549 cells expressing the FoxM1 S25A non-phosphorylation mutation, they were significantly decreased to the expression levels similar to those of the control group (FIG. 6B). The amounts of TGFB1 and VEGFA proteins in the medium cultured for 48 h with A549 cells (4×10⁴ cells/ml) expressing FoxM1 variants were measured by ELISA. The levels were significantly increased by 5 folds and 4 folds, respectively, in the S25E group compared to the control group (Mock), and in the S25A group, the levels were similar to those in the control group, indicating a strong decrease compared to S25E (FIG. 6C).

Next, an expression suppression system for suppressing FoxM1 expression was established to observe whether the FoxM1 phosphorylated point mutant (S25E) directly affects polarization to M2 macrophages. First, to inhibit the expression of mRNA of FoxM1, a pLKO-puro.1-hFoxM1 plasmid was produced using a pLKO-puro. 1 vector to construct shRNAs targeting nucleotide sequences at 709-729 positions in the human FoxMl mRNA sequence. The following sequences can be used as target sequences for human FoxMl shRNA production, and oligonucleotides with the following sequences can be used as primers for shRNA production. The gene accession number for human FoxM1 mRNA in PubMed is NM 001243088.2, and it contains 3507 bp. The following primers were used for shRNA production: 5′-ccgg-AGCAAGAGATGGAGGAAAAGG-ctcgag-CCTTTTCCTCCATCTCTTGCT-tttttg-3′ (forward primer) and 5′-aattcaaaaa-AGCAAG AGATGGAGGAAAAGG-ctcgag-CCTTTTCCTCCATCTCTTGCT-3′ (reverse primer). Then, after purifying and concentrating each lentivirus for expression of the produced FoxMl shRNA, it was constructed by infecting a lung cancer cell line, A549 cells expressing FoxM1, with the virus. The mRNA expression of M2 markers, CD163, CD206, and VEGFA, was examined after co-culturing lung cancer cells (4×10⁴ cells/ml) with suppressed FoxMl expression and THP-1 cells (1.2×10⁵ cells/ml) for 48 h. In the S25E cell line with suppressed FoxM1 expression, the mRNA expression of M2 markers CD163, CD206, and VEGFA was significantly decreased. When co-culturing with cancer cells expressing the FoxM1 variant S25E, it was confirmed that the M2 markers CD163, CD206, and VEGFA were all increased by 2 folds or more in THP1 cells compared to the control group (Mock_shCtrl) (FIG. 6D). It was indicated that FoxM1 S25E variant directly affected the polarization into M2 macrophages.

In addition, a broad macrophage marker, CD68, and a tumor-associated macrophage marker, CD163, were analyzed in the lung tissues of mice using immunostaining, and the expression of CD68 and CD163 markers was observed to be approximately 3 times higher in the lung tissues of mice injected with the cells over-expressing the FoxM1 phosphorylated point mutant (FIG. 6E, 6F). However, the expression of CD68 and CD163 markers was not significantly different from that of the Mock and was observed to be lower than that of the phosphorylated point mutant. Additionally, the increase of macrophages around the tumor was examined by immunoblot using lung tissue lysates. When observing the protein expression of CD68 and CD163, while the expression of these markers for tumor macrophages was significantly higher in the lung tissues of mice injected with the cancer cells overexpressing the phosphorylated point mutant (S25E), the expression of these markers for tumor-associated macrophages could be observed to be lower in the lung tissues of mice injected with the cancer cells overexpressing the non-phosphorylated point mutant (S25A) than that in the control group (FIG. 6G). Through the results of this study, it could be observed that the non-phosphorylated point mutant had the effect of inhibiting polarization into tumor-associated macrophages.

### Example 1.10

### Evaluation of Tumor Immune Evasion Capability by Proteins Containing the Phosphorylation and Non-Phosphorylation Point Mutations of FoxM1 (FIG. 7)

The cell viability was measured to investigate whether the FoxM1 phosphorylation and non-phosphorylation point mutations affected the viability of lung cancer cells, after co-culturing the lung cancer cells (A549) expressing the FoxM1 phosphorylation and non-phosphorylated point mutants and monocyte THP-1 cells. A549 cells (4×10⁴ cells/mL) and THP-1 cells were co-cultured at ratios of 1:0, 1:2, 1:4, and 1:6 for 48 hours, and after co-culturing for 48 h and washing off the THP-1 cells, the viability of A549 cells attached to the bottom was measured by an MTT assay.

As a result of the measurement, it could be observed that the viability of A549 cells expressing the FoxM1 phosphorylated point mutant (A549^{S25E}) gradually increased depending on the ratio of THP-1, and the viability was highest at a ratio of A549 cells: THP-1 cells of 1.6 (FIG. 7A). However, the viability of A549 cells expressing the FoxM1 non-phosphorylated point mutant (A549^{S25A}) was not changed compared to the control group.

To examine the mRNA expression levels of PD-1 and PD-L1, which are involved in solid tumor immune evasion, in THP-1 cells and the cells expressing the FoxM1 phosphorylated point mutant, the expression of PD-1 (CD279) mRNA, an immune evasion factor, was measured by qRT-PCR in THP-1 monocytes co-cultured with A549^{S25E} cells for 48 h (FIG. 7B). It was observed that the expression of mRNA of PD-L1 (CD274), an immune evasion factor, increased by 2 folds in the A549^{S25E} cells expressing the phosphorylated point mutant compared to the control group.

In addition, through the co-culture of the A549 cells expressing the FoxM1 S25E phosphorylated point mutant (A549^{S25E}) and Jurkat cells as T cells, it was tried to examine the effects on cancer immunity of T cells (FIG. 7D). It could be observed that the viability of A549^{S25E} cells (4×10⁴ cells/ml) expressing the phosphorylation mutant and co-cultured with Jurkat cells for 48 h was gradually increased by 1.2 folds, 1.3 folds, and 1.5 folds, as the ratio of Jurkat cells was increased to 1:0, 1:2, 1:4, and 1:6, while the viability of A549^{S25A} cells was significantly decreased compared to that of the A549 cells expressing WT or the phosphorylation mutant (FIG. 7D).

It indicated that A549^{S25E} cells might also affect the properties of T cells. To investigate the polarization into tumor-infiltrating T lymphocytes (TILs) by A549^{S25E} cells, the expression levels of CD25 and CD29, which were expressed in TILs, were analyzed by qRT-PCR after co-culturing Jurkat cells (1.2×10⁵ cells) and A549^{S25E} cells (4x104 cells) for 48 h. As a result, it was observed that there was a significant increase of 3 folds or more, in the expression of CD25 and CD29 in Jurkat cells co-cultured with A549^{S25E} cells. However, Jurkat cells co-cultured with A549^{S25A} cells showed that there was a significantly inhibiting effect compared to Jurkat cells co-cultured with A549 cells expressing WT or the phosphorylated point mutant (FIG. 7E). Under the previous experimental conditions, it was observed that there was an increase in the mRNA expression of IL6 and ILIA, which induced TILs polarization, and CD274, a tumor immune evasion factor, by 3.5 folds, 4.5 folds, and 5 folds, respectively, in the A549^{S25E} cells, whereas there was a significantly low expression of these factors in the A549^{S25E} cells, indicating an inhibitory effect (FIG. 7F). Additionally, to investigate the inhibitory effect of A549^{S25A} cells on tumor immune evasion by TAMs or TILs in the tumor microenvironment, the viability change was examined by an MTT assay analysis after co-culturing A549 cells expressing the FoxM1 phosphorylation or non-phosphorylated point mutant, THP-1 cells, and Jurkat cells at a ratio of 1:0:0, 1:6:0, 1:0:6, and 1:6:6 for 48 h. As a result, it was significantly increased in the FoxM1 phosphorylated point mutant (S25E), while it could be observed to be reduced in the FoxM1 non-phosphorylated point mutant (S25A) (FIG. 7G). That is, there was the inhibitory effect of the cells expressing non-phosphorylated point mutant on the tumor immune evasion by TAMs or TILs in the tumor microenvironment. Therefore, the present invention provided the inhibitory effects of the non-phosphorylated point mutant of FoxM1 on tumor immune evasion.

### Example 1.11

### Evaluation of Suppressive Efficacy on Cancer Cell Apoptosis and Motility by the Peptide Containing the Non-Phosphorylation Point Mutation of FoxM1 (FIG. 8)

To determine the inhibitory effects of a peptide comprising the non-phosphorylation point mutation of FoxM1 on the apoptosis and motility of cancer cells, the present inventors synthesized peptides by attaching TAT (YGRKKRRQRRR), a type of CPP (Cell-Penetrating Peptide) for infiltrating into cells, to three types of amino acid sequences, QNAPAETSEE (set #1, SEQ ID NO: 4), PAETSEEEPK (set #2, SEQ ID NO: 2), LPVQNAPAET (set #3, SEQ ID NO: 5), which contain the non-phosphorylation point mutation of FoxM1 (FIG. 8A). First, to determine the efficacy of the three peptides comprising the non-phosphorylation point mutation on cancer cell death, the activity of caspase-3 enzyme was measured by a caspase-3 assay to observe the apoptosis of cancer cells (FIG. 8B). After A549 lung cancer cells were treated with 5 μM of each of three types of the peptides, the cells were lysed. The cell lysates were incubated with a fluorescence substrate of caspase-3 (Ac-DEVD-AMC) for 2 hours, and then the fluorescence value (Ex. 380 nm/Emi. 430 nm) was measured using an electron spectrometer (SpectraMax M4 system). The measured fluorescence value indicated the caspase-3 activity and was represented as relative fluorescence units (RFU) to measure the apoptosis levels. As a result, all three peptides comprising the non-phosphorylation point mutation of FoxM1 showed apoptosis of cancer cells, especially, the higher efficacy could be observed when using peptides #1 and #3.

Moreover, to evaluate the effects and efficacy of the three peptides comprising the FoxM1 non-phosphorylation point mutation on the motility of cancer cells, the cell motility was measured by a migration assay (FIG. 8C). Specifically, 5×10⁴ cells of A549 lung cancer cells expressing the FoxM1 phosphorylated point mutant protein were seeded into an 8.0 μm, 24-well insert. Culture medium containing 10% serum (FBS) was added to a 24-well plate, and then the insert was placed into the plate. 0.5 ml of RPMI 1640 (10% FBS) containing 3 μg/ml of doxycycline was dispensed into the plate, and each of the three kinds of 5 μM of FoxM1 non-phosphorylated point mutant peptides was simultaneously treated. After 48 hours from cell seeding, 500 μl of 4% paraformaldehyde was dispensed into the plate, and the plate was washed three times with 1XPBS and stained with 0.05% crystal violet solution for 5 minutes. After 5 minutes, the plate was washed with 1XPBS five times, and the stained intensity was measured using the Odyssey infrared imaging system. When the stained intensity of the control group was set to 1, the relative stained intensity of each experimental group was represented in a graph. As a result of the study, the stained intensity was observed to be high in the FoxM1 phosphorylated point mutant over-expressed group and increased by 5 folds compared to the control group, and by treatment of each of the FoxM1 non-phosphorylated point mutant peptides, the stained intensity could be observed to decrease to approximately 20-40% (FIG. 8C). Hence, it was observed that treatment of the three peptides comprising FoxM1 non-phosphorylation point mutation not only inhibited the motility of the control group of A549 cancer cells, but also markedly suppressed the increased cell motility by FoxM1 phosphorylation. In particular, peptides #1 and #3 showed superior inhibitory effects on cancer cell motility.

From the above results, it is suggested that the peptides comprising the non-phosphorylation point mutation of FoxM1 showed the inhibitory effects on cell death of lung cancer cells and motility of cancer cells.

### Example 1.12

### Evaluation of the Effects of FoxM1 Expression on the Survival Rate of Patients by Cancer Types (FIG. 9)

To determine whether the expression level of FoxM1 was clinically significant in various cancers, the survival rate of patients based on the expression level of FoxM1 in patients with each cancer type was analyzed using big data analysis (FIG. 9A). The correlation between FoxM1 expression and the patient survival was assessed in various cancer types using Kaplan Meier plotter (https://kmplot.com/analysis). As a result of analyzing the expression of FoxM1 and the survival rate by 12 different types of cancer, it could be demonstrated that the survival rate of the patients with high expression of FoxM1 was significantly lower compared to that of the patients with low expression of FoxM1. Especially among the 12 types of cancer, the survival rate of patients with high expression of FoxM1 could be observed to be not only low also statistically significant (log rank P<0.05) in lung cancer (n=504, HR=2, log rank P-2.8e-05), breast cancer (n=947, HR=2.01,log rank P=0.0089), kidney cancer (n=530, HR-2.81, log rank P=1.9e-12), liver cancer (n=370,HR=2.07, log rank P=5.1e-05), thyroid cancer (n=353, HR=5.24, log rank P=6.9e-05), pancreatic cancer (n=69, HR=3.7, log rank P-0.022), esophageal cancer (n=19, HR=8.35, log rank P=0.03), endometrial cancer (n=542, HR=1.7, log rank P=0.011), sarcoma (n=152, HR=2.51, log rank P=0.00016) and adrenal pheochromocytoma (n=178, HR=6.98, log rank P-0.041). Additionally, the relative hazard ratio (HR) of the survival period in patients with high FoxM1 expression could be observed to be greater than 1.2 in testicular germ cell tumor (n=105, HR=2.76, log rank P=0.051) and cervical squamous cell carcinoma (n=174, HR=1.89, log rank P=0.098).

These results suggested that the FoxM1 expression affected the survival rate of patients with various cancer types.

In addition, the present inventors tried to evaluate the efficacy of the three peptides comprising the non-phosphorylation point mutation of FoxM1 produced previously on the apoptosis of cancer cells. Through the method described in FIG. 8B, the cell apoptosis of various cancer cells was examined by measuring the caspase-3 activity (FIG. 9B). After cervical cancer cells (HeLa) or liver cancer cells (Hep3B) were treated with 5 μM of each of the three peptides for 48 hours, the cells treated with the peptide were lysed and incubated with the fluorescence substrate of caspase-3 (Ac-DEVD-AMC) for 2 hours. Then, the fluorescence value (Ex. 380 nm/Emi. 430 nm) was measured by an electron spectrometer (SpectraMax M4 system). The measured fluorescence value indicated the caspase-3 activity and was represented as relative fluorescence units (RFU) to measure the apoptosis level. As a result, all three peptides comprising FoxM1 non-phosphorylation point mutation were observed to induce cancer cell apoptosis in cervical cancer cells (HeLa) or liver cancer cells (Hep3B), particularly the efficacy could be observed to be high for the peptide #1. Moreover, the efficacy of the peptides on apoptosis could be observed to be greater in liver cancer than in cervical cancer.

Therefore, it was indicated that the peptides comprising the non-phosphorylation point mutation of FoxM1 were capable of having apoptosis not only in lung cancer but also in other various cancer types.

### Example 1.13

### Evaluation Of Inhibitory Effects of the Peptides Comprising FoxM1 Non-Phosphorylation Point Mutation on Cancer Cell Motility and Macrophage Polarization (FIG. 10)

To evaluate the inhibitory effects of the peptides comprising FoxM1 non-phosphorylation point mutation on cancer cell motility and macrophage polarization in the metastatic environment, the experiments were performed using peptide #1, which expressed FoxM1 non-phosphorylation point mutation and showed the highest efficacy. To begin with, a peptide was synthesized by attaching the FITC-fluorescent material to the C-terminus of peptide #1 to determine whether the FoxM1 non-phosphorylated point mutant peptide could penetrate cells (FIG. 10A).

After 24 hours of treating lung cancer cells A549 with 5 μM of the FoxMl non-phosphorylated point mutant peptide, the FITC fluorescence was observed by a fluorescence microscope to determine whether the treated peptide penetrated the cells. As shown in FIG. 10B, the peptide was observed to penetrate well into the cells after 24 hours of treatment. In addition, under the metastatic conditions induced by 5 ng/ml of TGF-β, a change in the mRNA expression of epithelial-mesenchymal transition marker (EMT marker) by the treatment of the peptide penetrating the cells was observed by real-time PCR. In the group treated with TGF-β for 48 hours, the mRNA expression of the epithelial-mesenchymal markers, CDH2 and vimentin, was increased, whereas the treatment with the peptide reduced their expression. In contrast, in the TGF-β-treated group, the decrease in mRNA expression of the epithelial marker CDH1 was increased by the treatment with the peptide (FIG. 10C).

To determine the effects of treatment of the FoxM1 non-phosphorylated point mutant peptide on cancer cell motility in the metastatic environment treated with TGF-β, a migration assay was performed.

Specifically, 5×10⁴ cells of A549 lung cancer cells were seeded in an 8.0 μm, 24 well insert, and the inserts were placed in a 24-well plate after medium with 10% fetal bovine serum (FBS) was dispensed in the 24-well plate. For the positive control, 0.5 ml of RPMI1640 (10% FBS) containing 5 ng/ml of TGF-β was dispensed, and 5 μM of the non-phosphorylated point mutant peptide was treated simultaneously. After 48 hours from cell seeding, 500 μ [symbol] of 4% paraformaldehyde was dispensed into the plate, and the plate was washed three times with 1XPBS and stained with 0.05% crystal violet solution for 5 minutes. After 5 minutes, the plate was washed with 1XPBS five times, and the stained intensity was measured using the Odyssey infrared imaging system. When the stained intensity of the control group was set to 1, the relative stained intensity of each experimental group was represented in a graph.

As a result of the study, the stained intensity was observed to be high in the TGF-β-treated group and increased by 4 folds compared to the control group, and by treatment of the FoxM1 non-phosphorylated point mutant peptide, the stained intensity could be observed to decrease to similar to that in the control group (FIG. 10D). Hence, it could be found that the treatment of the FoxM1 non-phosphorylated point mutant peptide exhibited strong inhibitory effects on cell motility induced by TGF-β treatment.

The above results indicated that the FoxM1 non-phosphorylated point mutant peptide had inhibitory effects on metastatic properties in the metastatic environment induced by TGF-β treatment.

The present inventors studied the change of mRNA expression in epithelial-mesenchymal transition markers (EMT markers) by real-time PCR to investigate the change in the epithelial-mesenchymal transition markers by treatment of the FoxM1 non-phosphorylated point mutant peptide in the A549 lung cancer cells over-expressing the FoxM1 phosphorylated point mutant protein. After the cells with the overexpression system of the FoxM1 phosphorylated point mutant were treated with 3 μg/ml of doxycycline for 24 hours to induce the overexpression, the cells were treated with the FoxM1 non-phosphorylated point mutant peptide for 24 hours. Then, the levels of FoxM1 overexpression and mRNA expression of the epithelial-mesenchymal transition markers (EMT markers) were examined. As shown in FIG. 10E, it was observed that the FoxM1overexpression was well achieved, and that treatment with the peptide resulted in a decrease in the mRNA expression levels of mesenchymal transition markers CDH2 and vimentin, which had been increased by the overexpression of FoxM1. Conversely, it was observed that the decrease in mRNA expression of the epithelial marker CDH1 was reversed by the peptide treatment (FIG. 10E).

Furthermore, Under the conditions where the phosphorylated point mutant of FoxM1 was over-expressed in A549, the treatment with the FoxM1 non-phosphorylated point mutant peptide (5uM of FoxM1-S25A peptide) was observed to alter motility of cancer cells.

Specifically, 5×10⁴ cells of A549 lung cancer cells expressing the FoxM1 phosphorylated point mutant protein were seeded in an 8.0 μm, 24-well insert, and the inserts were placed in a 24-well plate after medium with 10% fetal bovine serum (FBS) was dispensed on the 24-well plate. 0.5 ml of RPMI 1640 (10% FBS) containing 3 μg/ml doxycycline was dispensed on the plate, and 5 μM of the non-phosphorylated point mutant peptide was treated simultaneously. After 48 hours from cell seeding, 500 μl of 4% paraformaldehyde was dispensed into the plate, and the plate was washed three times with 1XPBS and stained with 0.05% crystal violet solution for 5 minutes. After 5 minutes, the plate was washed with 1XPBS five times, and the stained intensity was measured using the Odyssey infrared imaging system. When the stained intensity of the control group was set to 1, the relative stained intensity of each experimental group was represented in a graph.

As a result of the study, the stained intensity value in the FoxM1 phosphorylated point mutant over-expressing group was observed higher in the FoxM1 phosphorylated point mutant over-expressing group and increased by 5 folds compared to the control group. The stained intensity could be observed to be decreased to approximately 50% by the treatment with the FoxM1 non-phosphorylated point mutant peptide (FIG. 10F). Therefore, it was observed that treatment with the FoxM1 non-phosphorylated point mutant peptide inhibited the cell motility that had been increased by FoxM1 phosphorylation.

Lastly, under conditions where the phosphorylated point mutant of FoxM1 was overexpressed in A549 cells, changes in the mRNA levels of STAT1, VEGFA, c-fos, IL6, and CD274 were observed to determine the inhibitory effects of the FoxM1 non-phosphorylated point mutant peptide (5 μM of FoxM1-S25A peptide) on tumor-associated macrophage polarization and the immune evasion ability of cancer cells.

After the cells with the overexpression system of the FoxM1 phosphorylated point mutant were treated with 3 μg/ml of doxycycline for 24 hours to induce the overexpression, the cells were treated with the FoxM1 non-phosphorylated point mutant peptide for 24 hours. Then, the mRNA expression levels of STAT1, VEGFA, c-fos, IL6, CD274 were examined. As shown in FIG. 10G, it was observed that treatment with the peptide resulted in a decrease in the mRNA expression levels of STAT1, VEGFA, c-fos, IL6, and CD274, which had been increased by the overexpression of FoxM1.

As the above results, it suggested that the FoxM1 non-phosphorylated point mutant peptide was capable of suppressing motility of lung cancer cells, polarization of tumor-associated macrophages, and immune evasion ability of cancer cells.

### Example 2

### Example 2.1

### Analysis for the Expression of FoxM1 and EMT Markers Under the Conditions where Cancer Metastasis was Induced by TGF-β

To observe changes in expression of FoxM1 during the epithelial-mesenchymal transition of lung cancer, the present inventors separately treated non-small cell lung cancer cell lines A549, NCI-H358, and NCI-H460 with TGF-β and observed EMT markers.

Specifically, the next day from seeding of 5×10⁴ cells/ml, the cells were treated with TGF-β for 48 hours to induce cancer metastasis, and then the activation of EMT could be confirmed by a marked increase in the amount of protein expression of mesenchymal markers N-cadherin, SNA11, and SNAI2 and a marked decrease in epithelial marker E-cadherin. In addition, it had been previously shown that binding of TGF-β protein to TGF-β family receptors led to phosphorylation of the receptors and activation of the SMAD complex, thereby activating the EMT program (Valcourt Ulrich, et al., Mol Biol Cell (2005) 16 (4) 1987-2002). It could also be observed that TGF-β treatment increased the expression of phosphorylated Smad-2 in this system. Under these conditions, we could observe that the protein expression of FoxM1 increased (FIG. 11).

These results indicated that the expression of FoxM1 increased in metastatic cancers.

### Example 2.2

### Evaluation of the Epithelial-Mesenchymal Transition Effect After Selection of Cells Expressing FoxM1 Wt Using Lentivirus

To examine the effects of FoxM1 protein on regulating epithelial-mesenchymal transition in cancer cells, cancer cells were cultured as follows to infect lung cancer cells with a lentivirus for expressing FoxM1 WT protein.

To establish a stabilized cell line expressing FoxM1 WT in A549 lung cancer cells, first, pLVX-Tet3G expressing lentivirus was infected into cells, and the infected cells were selected by treating 500 μg/ml of G418 for 5 days. The selected A549Tet3G cells were infected with lentiviruses expressing FoxM1 WT or Mock, and the stabilized cell lines were established by treating with 2 μg/ml of puromycin for 48 hours. After inducing the expression of FoxM1 WT by treating the established cells with 2 μg/ml of doxycycline, the protein expression was observed by immunoblot. As shown in FIG. 12A, it could be observed that the FoxM1 WT was well expressed and that the expression of mesenchymal markers N-cadherin and vimentin was increased by 2.2 folds and 2.8 folds, respectively and the expression of epithelial marker E-cadherin was decreased by 0.6 folds. In addition, as shown in FIG. 12B, it could be observed that the FoxM1 WT was well expressed by 4.4 folds, the expression of the mesenchymal markers N-cadherin and vimentin was increased by 1.5 folds and 1.7 folds, respectively, and the expression of the epithelial marker E-cadherin was reduced by 0.8 folds using real-time polymerase chain reaction.

These results suggested that the overexpression of FoxM1 WT promoted epithelial-mesenchymal transition in lung cancer cells.

### Example 2.3

### Evaluation of Effects of FoxM1 WT Protein on Promoting Metastasis

To examine the effects on motility of cancer cells in A549 lung cancer cells expressing FoxM1 WT protein, a migration assay was performed.

Specifically, 5×10⁴ cells of lung cancer cells A549 expressing control and FoxM1 WT were seeded in an 8.0 μm, 24 well insert, and the inserts were placed in a 24-well plate after medium with 10% fetal bovine serum (FBS) was dispensed in the 24-well plate. For the positive control, 0.5 ml of RPMI1640 (10% FBS) containing 5 ng/ml of TGF-β was added and used. After 72 hours from cell seeding, 500 μl of 4% paraformaldehyde was dispensed, washed three times with 1XPBS, and stained with 0.05% crystal violet solution for 5 minutes. After 5 minutes, the cells were washed five times with 1XPBS, and the staining intensity was measured using the Odyssey infrared imaging system. When the staining intensity of the control was set to 1, the relative staining intensity of each experimental group was calculated and displayed on a graph.

As a result of the study, the experimental group expressing FoxM1 WT protein showed the same improvement as the positive control group, the TGF-β treatment group, and it could be observed that the stained intensity was increased by 3 folds compared to the control group (FIG. 12C). From this result, it could be found that the FoxM1 WT protein promoted metastasis in lung cancer.

### Example 2.4

### Evaluation of Effects of FoxM1 WT Protein on Promoting Invasion

To examine the effects on invasion of cancer cells in A549 lung cancer cells expressing FoxM1 WT protein, an invasion assay using Matrigel was performed.

Specifically, Matrigel was completely solved at 4° C. for 16-20 hours and diluted to 1 mg/ml in cold serum free RPMI 1640 (4° C.). 100 μl of Matrigel mixture (1 mg/ml) was added into 8.0 mm 24-well insert and solidified in a 37° C. incubator for 12-20 hours. A549 lung cancer cells expressing Mock or the experimental group FoxM1 WT protein were dispensed on the solidified Matrigel insert with 1×10⁵ cells/well diluted in serum free RPMI 1640 (36° C.), and 0.5 ml/well of 36° C. of warm RPMI 1640 (10% FBS) were dispensed. For the positive control group, 0.5 ml of 36° C. RPMI 1640 (10% FBS) containing 5 ng/ml of TGF-β were dispensed. Then, once in 3 days, the medium was changed, and the level of invasion was observed. On 7th day when the invasion of cancer cells was observed to be sufficiently occurred, the medium was removed, washed with 1XPBS, and the cells inside the insert were scraped with a cotton swab and washed with 1X PBS to remove any remaining cells and Matrigel inside the insert. 500 μl of 4% paraformaldehyde was dispensed into 24 wells where the outer surface of the insert was placed, incubated for 5 minutes at room temperature, washed three times with 1X PBS, and stained with 0.05% crystal-violet solution for 5 minutes. After 5 minutes, they were washed 5 times with 1XPBS, and the degree of staining was dissolved in DMSO, and the wavelength was measured at 590 nm. When the absorbance of the control group was set to 1, the relative absorbance of each experimental group was calculated and displayed on a graph.

As a result of the study, in the experimental group expressing the FoxM1 WT protein, it could be observed that the absorbance value was increased by approximately 20 folds compared to the control group, which was the same as the positive control group treated with TGF-beta (FIG. 12D). Therefore, it could be found that the FoxM1 WT had an outstanding effect in promoting the invasion of lung cancer cells.

### Example 2.5

### Evaluation of Polarization Ability of Macrophages by FoxM1 WT Protein

To determine whether the cancer cells expressing the FoxM1 WT protein affected polarization of macrophage cells in the tumor microenvironment, after co-culturing human monocyte THP-1 cells and FoxM1 WT protein over-expressed cells, M1 and M2 markers were observed in THP-1 cells. After co-culturing the FoxM1 WT protein over-expressed cancer cells (4×10⁴ cells/ml) and THP-1 cells (1.2×10⁵ cells/ml) for 48 h, the mRNA expression of M1 markers INOS and IL12B was not altered, while the mRNA expression of all the M2 markers IL10, CD163, CD206, TGFB1, and VEGFA was increased in the FoxM1 WT (FIG. 13A). Among them, CD206, TGFB1, and VEGFA had been known as markers for M2d-tumor-associated macrophages (M2d-TAM) (JAYASINGAM, Sharmilla Devi, et al., Frontiers in Oncology (2020) 9:1512.). Since the expression of M2d-tumor-associated macrophage markers was observed to be high in the THP-1 cells co-cultured with the A549 cells expressing the FoxM1 WT protein, it could be found that THP-1 cells were polarized into M2d-TAM by the expression of FoxM1 WT.

In addition, under the previous experimental conditions, after co-culturing human monocyte THP-1 cells and the FoxM1 WT protein over-expressed cell groups for 48 hours, the results of analyzing the mRNA expression level of M2d-inducing factors IL4, IL6, IL10, and VEGFA in A549 cells expressing FoxM1 WT protein were observed to be increased (FIG. 13B). The results of analyzing the amounts of TGFB1 and VEGFA proteins in the medium in which A549 cells (4×10⁴ cells/ml) expressing FoxM1 WT protein were cultured for 48 hours by ELISA were observed to be markedly increased in the FoxM1 WT compared to the control group (Mock) (FIG. 13C).

Furthermore, CD68, a marker for a broad range of macrophages, and CD163, a marker for tumor-associated macrophages, were analyzed in lung tissues of mice using immunostaining. It was determined that the expression of both CD68 and CD163 markers was increased in the lung tissues of mice injected with the FoxM1 WT protein over-expressed cells (FIG. 13D). Through the present results, it could be observed that there was an effect of promoting the polarization of M2 tumor-associated macrophages in lung cancer cells expressing the FoxM1 WT protein.

### Example 2.6

### Evaluation of Inhibitory Effects of FoxM1 shRNA on Epithelial-Mesenchymal Transition

To determine the inhibitory effects on the mRNA expression of FoxMI, shRNA and a lentivirus comprising thereof were produced.

Specifically, to suppress the mRNA expression of FoxM1, primers were designed as follows to construct shRNA targeting the nucleotide sequences of positions 187-207 (target #1) and 709-729 (target #2) of human FoxMl mRNA (Human FoxMl mRNA [NM_001243088.2]).

The forward primer targeting the nucleotide sequence at positions 187-207 (target #1) of the FoxMl mRNA sequence was 5′-ccgg-CAT CAG AGG AGG AAC CTA AGA-ctcgag-TCT TAG GTT CCT CCT CTG ATG-tttttg-3′, and the reverse primer was 5′-aattcaaaaa-CAT CAG AGG AGG AAC CTA AGA-ctcgag-TCT TAG GTT CCT CCT CTG ATG-3′. In addition, 5′-ccgg-AGC AAG AGA TGG AGG AAA AGG-ctcgag-CCT TTT CCT CCA TCT CTT GCT-tttttg-3′ was used as the forward primer targeting the nucleotide sequence at positions 709-729 (target #2) of the FoxMl mRNA sequence, and 5′-aattcaaaaa-AGC AAG AGA TGG AGG AAA AGG-ctcgag-CCT TTT CCT CCA TCT CTT GCT-3′ was used as the reverse primer. Based on this, the pLKO-puro.1-shFoxM1 plasmid was constructed using the pLKO-puro.1 vector. This was expressed through transfection of HEK293 cells with pHR'-CMV-VSVG and pHR'-CMV-deltaR8.2, and the culture medium of the cells was collected to produce lentivirus. The lentivirus was concentrated using a centrifuge. To examine the virus expression, A549 cells were cultured at 5×10⁴ cells/ml, and the next day, the lentivirus was added at 20 μ/well in infection buffer (10 mM HEPES, 1 mg/ml polybrene) to infect cancer cells. After 24 hours, the cells infected with shFoxM1 were selected by treating with puromycin at 2 μg/ml for 48 hours.

Initially, the present inventors examined the mRNA and protein expression levels of FoxM1 in the cells infected with each FoxM1 shRNA and selected by treatment with 2 μg/ml of puromycin for 48 hours to determine the effects of FoxM1 shRNA on suppressing FoxM1 expression.

As shown in FIG. 14A and 14B, the expression of FoxM1 mRNA was reduced in the A549 lung cancer cells infected with each FoxMl shRNA (shFoxM1) compared to the cells infected with the control shRNA (shCtrl). It indicated that the expression of FoxM1 was suppressed. In particular, it was observed that the shRNA targeting the nucleotide sequence at position 709-729 (target #2) in the FoxMl mRNA sequence had the best effect in suppressing the expression of FoxM1.

Therefore, the present inventors used the shRNA targeting the nucleotide sequence at position 709-729 (target #2) in the FoxMl mRNA sequence in the following FoxM1 expression suppression assays.

The present inventors performed a migration assay to determine whether suppression of FoxM1 expression by FoxMl shRNA treatment inhibited lung cancer cell migration. Specifically, the A549 lung cancer cells were separately infected with the control shRNA (shCtrl) and FoxM1 shRNA (shFoxM1) viruses. The next day, after treatment of 2 μg/ml of puromycin for 48 hours, the selected shFoxM1 expressed cells were seeded on a 6-well plate at 2×10⁵ cells/ml, and 24 hours later, a scratch was made at regular intervals using a pipette tip. After scratching, the gaps and motility of the cells were observed using a microscope at 24-hour intervals, and the extent to which the gaps were restored was measured as the distance between cells through cell photography under a microscope. The measured value in the control group was set to 100%, and the relative migration distance was calculated and displayed on a graph as % (FIG. 14C).

The relative migration distance (%)=The measured value in the experimental group X 100/The measured value in the control group

As a result of the study, the suppression of FoxM1 expression by FoxM1 shRNA treatment reduced their metastatic potential of lung cancer cells, especially, at 72 hours, when the motility of the control group was set to 0%, the motility decreased by approximately −20% or more (FIG. 14D). Hence, it could be found that suppression of FoxM1 expression by FoxM1 shRNA has a remarkable inhibitory effect on the metastatic potential of cancer cells.

The present inventors observed changes in mRNA and protein expression of epithelial-mesenchymal transition markers and related factors by suppression of FoxM1 mRNA expression through the FoxM1 shRNA treatment in cancer metastasis induced by TGF-β treatment.

Under the conditions where the established cell line suppressing FoxM1 was treated with 5 ng/ml of TGF-β for 48 hours to induce cancer metastasis, the changes in the expression of epithelial-mesenchymal transition-related factors CDH1 and CDH2 by real-time PCR (FIG. 14E).

The A549 lung cancer cells infected with the FoxM1 shRNA (shFoxM1) showed a decrease in mRNA expression of FoxM1 compared to the cells infected with the control shRNA (shCtrl), which indicated that the expression of FoxM1 was inhibited. Even under the conditions treated with TGF-β for inducing cancer metastasis, it was observed that the expression of FoxM1 was inhibited (FIG. 14E). It was observed that the decrease in the expression of CDH1, an epithelial marker, by TGF-β treatment was suppressed by the suppression of FoxM1 expression by shFoxM1 treatment, and that the increase in the expression of CDH2, a mesenchymal marker, by TGF-β treatment was suppressed by the suppression of FoxM1 expression (FIG. 14E).

### Example 2.7

### Effects on Inducing Apoptosis by FoxM1 shRNA Treatment

To observe the degree of apoptosis of cancer cells in A549 cells by suppression of FoxM1 expression by shFoxM1 treatment, a caspase-3 assay was performed to measure the activity of caspase-3 enzyme (FIG. 14F). A549 cells were infected with viruses expressing shCtrl, shFoxM1#187, or shFoxM1#709 for 48 hours, and then the cells were selected with puromycin for 48 hours, collected, and lysed. After reaction with the fluorescent substrate of caspase-3 (Ac-DEVD-AMC) for 1 hour, the fluorescence value (Ex. 380 nm/Emi. 430 nm) was measured using an electron spectrometer (SpectraMax M4 system). The measured fluorescence value was represented as relative fluorescence units (RFU), as caspase-3 activity. When the measured value of the control group was set to 1, the degree of apoptosis was calculated by calculating the degree of relative measured value in each experimental group. As a result, it was observed that the caspase-3 activity increased by 5 folds or more in the shFoxM1#709 cells and by 2 folds or more in the shFoxM1#187 cells compared to the control group that was the group treated with shRNA (shCtrl). Therefore, it could be found that the inhibition of FoxM1 expression by shFoxM1 treatment induced and increased apoptosis.

### Example 2.8

### Evaluation of Polarization Ability of Macrophages by Foxml ShRNA in the Phosphorylated Point Mutant of FoxM1

The present inventors observed whether suppression of FoxM1 expression by FoxM1 shRNA treatment reduced FoxM1 expression in the phosphorylated point mutant of FoxM1.

Specifically, the A549 lung cancer cells expressing the FoxM1 phosphorylated point mutant (S25E) were infected with the control shRNA (shCtrl) and FoxM1 shRNA (shFoxM1) viruses. When the mRNA expression of FoxM1 was observed in the infected cells through real-time polymerase chain reaction (real-time PCR), it could be observed that the FoxMl shRNA treatment decreased the FoxM1 expression inhibition in the phosphorylated point mutant of FoxM1 (FIG. 15A).

Furthermore, after co-culture of cancer cells (4×10⁴ cells/ml) treated with FoxMl shRNA and human monocyte THP-1 cells (1×10⁵ cells/ml) for 48 hours, the results of analyzing the mRNA expression level of M2 marker factors CD163, CD206, and VEGFA in THP-1 could be observed to be markedly decreased in the FoxM1 phosphorylated point mutant expressed cell group (S25E) treated with the FoxM1 shRNA (FIG. 15B).

In addition, the mRNA expression of IFITM1 was observed by real-time polymerase chain reaction (Real-time PCR) in the A549 lung cancer cells expressing the FoxM1 phosphorylated point mutant (S25E) separately infected with the control shRNA (shCtrl) and the FoxM1 shRNA (shFoxM1) viruses. As a result of suppressing the FoxM1 expression by the FoxM1 shRNA treatment in the phosphorylated point mutant of FoxM1, the mRNA expression of IFITM1 could be observed to be markedly decreased (FIG. 15C).

### Example 2.9

### Evaluation of the Effect of Inhibiting Epithelial-Mesenchymal Transition and Macrophage Polarization Ability by Treatment with Thiostrepton, a Foxm1 Inhibitor, in Cells Overexpressing the Phosphorylated Point Mutant of FoxM1

The present inventors observed whether the inhibition of FoxM1 expression by treatment with Thiostrepton, a FoxM1 inhibitor, suppressed epithelial-mesenchymal transition and macrophage polarization ability in cells overexpressing the phosphorylated point mutant protein of FoxM1.

Specifically, when A549 lung cancer cells were treated with Thiostrepton, a FoxM1 inhibitor, at a concentration of 5 μM for 48 h in the control group (Mock) and cells expressing FoxM1 phosphorylated point mutant (S25E), it was observed through real-time polymerase chain reaction (real-time PCR) that the expression of mesenchymal markers CDH2, vimentin, SNAI1, and SNAI2, which were factors related to the epithelial-mesenchymal transition, was significantly reduced in the group treated with Thiostrepton, a FoxM1 inhibitor (FIG. 16A).

In addition, under the same experimental conditions as above, it could be observed that the expression of M2 inducing factors IL6 and VEGFA and an immune evasion factor CD274 (PD-L1) was significantly reduced in the group treated with Thiostrepton, a FoxM1 inhibitor (FIG. 16B).

From the above results, in this study, it was found that epithelial-mesenchymal transition, macrophage polarization ability, and immune evasion ability of cancer cells were suppressed by treatment with Thiostrepton, a FoxM1 inhibitor, in cells overexpressing the phosphorylated point mutant of FoxM1.

1. A pharmaceutical composition for treating cancer, comprising a polypeptide in which the 25th amino acid, Ser of SEQ ID NO: 1 is substituted with a non-phosphorylated amino acid.

2. The pharmaceutical composition according to claim 1, wherein the non-phosphorylated amino acid is Gly, Ala, Val, Ile, Leu, Met, Phe, Trp, Asn, Gln, Cys, Pro, Arg, His, or Lys.

3. The pharmaceutical composition according to claim 1, wherein the cancer is bone cancer, blood cancer, lung cancer, small cell lung cancer, non-small cell lung cancer, squamous cell carcinoma, adenocarcinoma, large cell lung cancer, liver cancer, pancreatic cancer, skin cancer, head and neck cancer, skin or intraocular melanoma, uterine cancer, ovarian cancer, rectal cancer, anal region cancer, gastric cancer, colorectal cancer, breast cancer, prostate cancer, endometrial cancer, sarcoma, pheochromocytoma adrenal tumor, testicular germ cell tumor, cervical cancer, carcinoma of sexual and reproductive organs, Hodgkin's disease, esophageal cancer, small intestinal cancer, endocrine cancer, thyroid cancer, parathyroid cancer, adrenal cancer, soft tissue sarcoma, bladder cancer, kidney cancer, renal cell carcinoma, renal pelvic carcinoma, central nervous system (CNS) neoplasm, neuroectodermal tumor, spinal tumor, glioma, meningioma or pituitary adenoma.

4. The pharmaceutical composition according to claim 1, wherein the pharmaceutical composition induces cancer cell death, or inhibits at least one selected from the group consisting of growth, migration, invasion and metastasis of cancer cells.

5. A polypeptide, comprising a polypeptide, comprising the 24th amino acid, Pro to the 27th amino acid, Thr of SEQ ID NO: 1, and comprising 10 or more consecutive amino acids, in which the 25th amino acid, Ser is substituted with a non-phosphorylated amino acid.

6. The polypeptide according to claim 5, wherein the polypeptide comprises an amino acid sequence represented by SEQ ID NO: 2, 4 or 5.

7-18. (canceled)

19. A method for treating cancer, comprising administering a polypeptide comprising the 24th amino acid, Pro to the 27th amino acid, Thr of SEQ ID NO: 1, and comprising 10 or more consecutive amino acids, in which the 25th amino acid, Ser is substituted with a non-phosphorylated amino acid, into a subject in need of treating cancer.

20. The method for treating cancer according to claim 19, wherein the polypeptide comprises an amino acid sequence represented by SEQ ID NO: 2, 4 or 5.

21. The method for treating cancer according to claim 19, wherein the cancer is bone cancer, blood cancer, lung cancer, small cell lung cancer, non-small cell lung cancer, squamous cell carcinoma, adenocarcinoma, large cell lung cancer, liver cancer, pancreatic cancer, skin cancer, head and neck cancer, skin or intraocular melanoma, uterine cancer, ovarian cancer, rectal cancer, anal region cancer, gastric cancer, colorectal cancer, breast cancer, prostate cancer, endometrial cancer, sarcoma, pheochromocytoma adrenal tumor, testicular germ cell tumor, cervical cancer, carcinoma of sexual and reproductive organs, Hodgkin's disease, esophageal cancer, small intestinal cancer, endocrine cancer, thyroid cancer, parathyroid cancer, adrenal cancer, soft tissue sarcoma, bladder cancer, kidney cancer, renal cell carcinoma, renal pelvic carcinoma, central nervous system (CNS) neoplasm, neuroectodermal tumor, spinal tumor, glioma, meningioma or pituitary adenoma.

22. The method for treating cancer according to claim 19, wherein the pharmaceutical composition induces cancer cell death, or inhibits at least one selected from the group consisting of growth, migration, invasion and metastasis of cancer cells.

23-36. (canceled)


SEQUENCE LISTING (11 sequences)

SEQ ID NO: 1 - protein, 748 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCDGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKQQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 2 - protein, 10 aa, Synthetic construct
PAETSEEEPK

SEQ ID NO: 3 - DNA, 2247 bp, Homo sapiens
  source 1..2247: mol_type = other DNA
atgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagcagcagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggagaaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtag

SEQ ID NO: 4 - protein, 10 aa, synthetic construct
PAETSEEEPK

SEQ ID NO: 5 - protein, 10 aa, synthetic construct
LPVQNAPAET

SEQ ID NO: 6 - DNA, 3326 bp, Homo sapiens
  source 1..3326: mol_type = other DNA
ggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgcagatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagcagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagcttatttccttagatcattatccagagactgccagaaggtgggtaggatgacctggggtttcaattgacttctgttccttgcttttagttttgatagaagggaagacctgcagtgcacggtttcttccaggctgaggtacctggatcttgggttcttcactgcagggacccagacaagtggatctgcttgccagagtcctttttgcccctccctgccacctccccgtgtttccaagtcagctttcctgcaagaagaaatcctggttaaaaaagtcttttgtattgggtcaggagttgaatttggggtgggaggatggatgcaactgaagcagagtgtgggtgcccagatgtgcgctattagatgtttctctgataatgtccccaatcataccagggagactggcattgacgagaactcaggtggaggcttgagaaggccgaaagggcccctgacctgcctggcttccttagcttgcccctcagctttgcaaagagccaccctaggccccagctgaccgcatgggtgtgagccagcttgagaacactaactactcaataaaagcgaaggtggacaaaaaaaaaaaaaaaaaa

SEQ ID NO: 7 - DNA, 21 bp, synthetic construct
  source 1..21: mol_type = other DNA
catcagaggaggaacctaaga

SEQ ID NO: 8 - DNA, 21 bp, synthetic construct
  source 1..21: mol_type = other DNA
tcttaggttcctcctctgatg

SEQ ID NO: 9 - DNA, 21 bp, synthetic construct
  source 1..21: mol_type = other DNA
agcaagagatggaggaaaagg

SEQ ID NO: 10 - DNA, 21 bp, synthetic construct
  source 1..21: mol_type = other DNA
ccttttcctccatctcttgct

SEQ ID NO: 11 - protein, 748 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ